(19) United States
(12) Patent Application Publication
(10) Pub. No.: US 2022/0283190 A1
(43) Pub. Date: Sep. 8, 2022

(54) METHODS AND DEVICES FOR DETECTION OF COAGULATION IMPAIRMENT

(71) Applicant: Galit H. Frydman, Boston, MA (US)

(72) Inventor: Galit H. Frydman, Boston, MA (US)

(21) Appl. No.: 17/195,615

(22) Filed: Mar. 8, 2021

(51) Int. Cl. G01N 33/86; B01L 3/00

(52) U.S. Cl. CPC G01N 33/86; B01L 3/5027; B01L 2300/088; G01N 2333/974; G01N 2333/96463; G01N 2333/96452; G01N 2333/9645; G01N 2333/96458

(57) ABSTRACT

Provided are methods and devices for evaluating coagulation, including the identification of a coagulation impairment such as a factor deficiency or the presence of a factor inhibitor. In various embodiments, the methods and devices measure coagulation of a sample in response to the addition of one or more coagulation factors, added at various concentrations to portions of the sample. Such coagulation measurements can be evaluated to accurately profile coagulation impairments of the sample. In additional various embodiments, point-of-care or bedside testing with a convenient, microfluidic device can be used by minimally trained personnel.

### BACKGROUND

The coagulation system is a delicate balance between hemorrhage and thrombosis. There are many disease conditions and clinical situations (such as, for example, cancer, auto-immune disease, infection, trauma, surgery, heart disease, and drug treatments), that can cause a disruption of this balance and result in a patient having severe, and in some cases even life-threatening, bleeding or clotting events. In some cases, a patient may suffer from coagulopathy, which can be congenital and/or hereditary, or acquired. Coagulopathy may result from a deficiency in a coagulation factor; examples of coagulation factor deficiencies that may result in a coagulopathy include a deficiency in Factor VIII, deficiency in Factor IX, and deficiency in Factor XI (e.g., Hemophilia A, B, and C, respectively). While hemophilia is most commonly known as a congenital and/or hereditary disease, there are also acquired causes of hemophilia, such as the development of anti-factor autoantibodies (e.g., Acquired Hemophilia A or AHA). It is possible to have coagulation factor deficiencies and/or inhibitors (such as autoantibodies) to any of the coagulation factors, although deficiencies of and inhibitors to FVII, FXII, and FXIII tend to be less common than are deficiencies of and inhibitors to other coagulation factors. Deficiencies of or inhibitors to coagulation factors may lead to prolongation in bleeding times and adverse bleeding events; whether such consequences occur often depends on the level of deficiency or on the inhibitor concentration. While the most common treatment for deficiency of any given factor is factor replacement (via administration of the specific factor that is deficient), there are new treatments being pursued that trigger the body to produce the factor on its own. Such treatments include bypassing agents (e.g., emicizumab) and gene therapies (including DNA- and RNA-based therapies).

While congenital factor deficiencies range from mild to severe, the development of auto-antibodies to coagulation factors can rapidly lead to severe, life-threatening bleeding events. Anti-factor antibodies occur in up to 30% of all hemophilia patients treated with factor replacement and can also occur spontaneously in adults who have no factor deficiency and are not being treated with factor replacement. Clinical presentation for anti-factor antibody development is usually in the emergency room, where a patient may present with moderate to severe spontaneous bleeding. The treatment for anti-factor antibodies (a type of factor inhibition) can be different from the treatment for factor deficiency, due to the supra-physiologic levels of that factor that are required to overcome the presence of the factor inhibitor, depending on the concentration and inhibition activity of the inhibitor (in this case, the anti-factor antibody); this requirement for supra-physiologic factor levels usually necessitates the administration of high levels of factor replacement or of bypassing agents, where inactivated or activated coagulation factors downstream of the point of inhibition are administered in the event of adverse bleeding. Such inactivated or activated coagulation factors include, for example, activated Factor VII (FVIIa) or anti-inhibitor Coagulant Complex (FEIBA®), which contains Factors II, VIII, IX, and X, as well as FVIIa. It is imperative for a clinician to appropriately diagnose whether a patient is factor deficient and/or has factor inhibitors (such as anti-factor antibodies), as the treatment for factor inhibitors (e.g., administration of FVIIa or FEIBA®) involves activated bypassing factors that could result in embolic and thromboembolic events if not administered appropriately. The risk of embolic and thromboembolic events also makes the dosing and duration of administration of these bypassing agents very important clinical considerations.

In addition, an individual may suffer from a coagulation factor abnormality, such as an abnormality that results from a genetic mutation. An abnormality may or may not result in changes to coagulation factor function. In cases where an abnormality results in a decrease in coagulation factor function, such decrease in factor function may also result in a prolongation in bleeding time and/or adverse bleeding events. In such cases where an abnormality results in a decrease in coagulation factor activity, the abnormality causes a factor deficiency. In other cases, an abnormality to a coagulation factor, such as the abnormality caused by a mutation in Factor V in patients with Factor V Leiden (FVL) thrombophilia, results in an increase in blood clotting; in the case of FVL, the mutation results in resistance to Activated Protein C (APC), a circulating anticoagulant and inhibitor to Factor Va and VIIIa, and thereby causes an increase in blood clotting.

There are also conditions that result in transient changes in coagulation factor concentrations. Many coagulation factors are acute phase proteins, meaning that their concentration may increase or decrease during an inflammatory response. For example, Factor I (fibrinogen) and Factor VIII are positive acute phase proteins and their concentrations increase with inflammation, while antithrombin III (AT or ATIII) is a negative acute phase protein, as its concentration decreases with inflammation. These dynamic changes in coagulation factor levels that can happen during, by way of example, inflammation, stress, or pregnancy, are important considerations when assessing an individual's coagulation phenotype and selecting or monitoring patient treatment.

Current clinical tests used to detect and identify specific coagulation factor deficiencies or inhibition require a series of specialty coagulation tests, including, but not limited to, activated partial thromboplastin time (aPTT) assay, complex dilute plasma tests for identifying factor deficiency, and Bethesda assays (including modifications thereof) for detecting and quantifying anti-factor auto-antibodies. For a patient with an undiagnosed factor deficiency or inhibition, diagnosis usually requires multiple specialty tests (which are typically performed by specialty coagulation laboratories) and the simultaneous interpretation of these multiple test results. For patients with anti-factor antibody development, there are no rapid, non-specialty or point-of-care tests currently available to diagnose these patients and to differentiate factor deficiency from factor inhibition, such as inhibition caused by anti-factor antibodies. Consequently, time-delays in delivering appropriate treatment to these patients can increase morbidity and mortality. In particular, while the typical treatment for factor deficiency is factor replacement, patients with elevated levels of factor inhibitors (such as anti-factor antibodies) may require supra-physiologic levels of factors to overcome the inhibitor or may require bypassing agents to achieve rapid hemostasis. To further complicate the clinical picture, if a bypassing agent is given to a patient with a coagulation factor deficiency but with no factor inhibitors (such as anti-factor antibodies), such patient may be at an increased risk of an embolic or thromboembolic event due to the inappropriate administration of activated bypassing coagulation factors.

Two primary classes of coagulation tests are used to evaluate coagulation factor deficiency and inhibition: “activity” tests and “functional” tests. In addition to these tests, genetic analysis may be used to evaluate whether a patient has a coagulation factor mutation, and mass-spectrometry, western blots, and ELISAs can be used to quantify coagulation factor concentrations (although none of these quantitative tests is commonly used for patient treatment monitoring, as they are not functional- or activity-based tests and, therefore, have limited clinical value).

Currently available coagulation factor activity tests are indirect-activity tests that utilize a chromogenic read-out and require platelet-poor plasma (PPP). For example, one way to perform a Factor IX (FIX) chromogenic activity assay is to take a patient's plasma sample (FIX deficient) and add in Factor XIa, Factor VIIIa, calcium, and phospholipids. The resultant Factor Xa, which is generated through the complexing of Factor IXa (present at a sub-physiologic level or with sub-physiologic activity in a FIX-deficient sample) and Factor VIIIa, is then quantified using a chromogenic substrate that binds to and detects Factor Xa. Similarly, one way to perform the Factor VIII (FVIII) chromogenic activity assay is to take a patient's plasma sample (FVIII deficient), add in Factor XIa to activate coagulation, and add Factor X in excess, Factor IIa, calcium, and phospholipids. Factor Xa is then generated and quantified with the chromogenic substrate. These protocols, which use the Factor Xa chromogenic assay as an indirect method of detecting deficiencies in FIX or FVIII, are cumbersome and require the simultaneous addition of multiple coagulation factors in order to create optimal assay conditions. In essence, these tests attempt to isolate the coagulation factor in question (in these two examples, FIX or FVIII) by adding multiple exogenous coagulation factors to a single plasma sample in order to identify a deficiency in that factor. However, these tests are not able to assess how the patient's other endogenous coagulation factors affect the generation of Factor Xa. For example, a variety of comorbidities may result in changes to various endogenous factor levels and may play a role in the coagulation phenotype of a patient. See, e.g., Brummel-Ziedins et al., PLoS One 7:e29178 (2012). But because the chromogenic tests require the addition of coagulation factors to the patient's sample, the tests are not designed to and cannot determine how a patient's elevated level of another factor affects the patient's coagulation phenotype.

Coagulation factor functional assays are also cumbersome and cannot assess a patient's full coagulation phenotype. For example, in a 2-stage FVIII assay, the patient's plasma sample (FVIII deficient) is adsorbed prior to testing (this adsorption removes endogenous Factors II, VII, IX, and X from the patient's sample), exogenous Factor XIa is added to activate coagulation, and excess Factor X and Factor V are added, resulting in activation of Factor X. Next, part of this plasma mixture is added to normal plasma (which is plasma having physiologic levels of coagulation factor activity) and the time to clot is recorded, wherein the clotting time is presumed to depend on the level of Factor Xa generated during the first part of the test. In this assay, not only are multiple exogenous factors added to the patient sample, but the normal plasma also provides about 50% of all of the coagulation factors present in the total sample being tested at step two. The addition of normal plasma is imperative in this clotting assay due to the adsorption treatment of the patient's plasma to remove Factor II (in other words, the plasma would not be able to clot without the addition of the normal plasma). These 2-stage tests remove several of the patient's key coagulation factors via adsorption and then add them back via the addition of the normal plasma to enable clot formation. These tests are therefore optimized to isolate an aberrancy in the factor being tested (in this example, FVIII) and do not take into account any other endogenous factor levels, changes, or abnormalities.

There are also functional tests based on the modification of the prothrombin time (PT) or activated partial thromboplastin time (aPTT) assays. In the 1-step PT or aPTT assay, a patient's plasma, at various dilutions, is added to plasma that is deficient in a single factor (the reference plasma). First, a set of serial dilutions is performed for both the reference plasma and the patient's plasma to obtain a baseline of the clotting time at various dilutions. Second, these clotting time curves are plotted on the same graph, and the factor activity is derived by evaluating where the reference plasma curve intercepts the patient's plasma curve using a series of vectors drawings. This assay, however, does not assess the degree to which the factor in question contributes to changes in clotting time, as the assay simply compares clotting times to a reference. Additionally, because the aPTT assay is typically phospholipid-based, plasma samples from patients with lupus anticoagulant may record prolongations in the aPTT clotting time (depending on the aPTT reagents used), indicating a clotting factor deficiency that may not, in fact, be present.

There are also tests that are used for the detection and quantification of anti-factor antibodies (inhibitors), but these tests are similarly cumbersome and utilize the simultaneous addition of multiple exogenous factors. The most basic screening test for the presence of inhibitors entails mixing the patient's plasma with normal plasma and then comparing the resultant uncorrected, prolonged aPTT to both the normal plasma's aPTT and the patient's baseline aPTT. Once again, depending on the aPTT reagents used, if the patient's sample has lupus anticoagulant, this assay may mislead a clinician to suspect the presence of a factor inhibitor when the result instead may be caused by the presence of lupus anticoagulant.

The Bethesda Assay (BA) is the most widely used test to quantify inhibitor presence and multiple variations have been developed in an attempt to increase the assay's sensitivity and specificity. In the BA protocol for FVIII deficiency, for example, two assays are performed in parallel: diluted patient plasma is mixed with an aliquot of normal plasma in a first test, while an aliquot of normal plasma is mixed with buffer diluent in a second test. Both of these mixtures are incubated for 120 minutes and the residual FVIII activity is then calculated using a chromogenic or clot-based approach, as described above, or using the one-step aPTT assay. In the Nijmegen modification of the BA, diluted patient plasma is mixed with an aliquot of normal plasma for one test and FVIII-deficient control plasma is mixed with an aliquot of normal plasma for another test. Both of these mixtures are then incubated for 120 minutes and residual factor activity is then calculated using a chromogenic or clot-based approach, such as the one-step aPTT. In patients receiving FVIII replacement therapy, heat treatment is sometimes used in order to separate the inhibitor-factor complex to allow for complete inhibitor quantification; however, this heat treatment often results in a decrease in inhibitor activity due to heat inactivation. Furthermore, in all of these approaches, normal plasma is added to the patient plasma, replacing and/or overcoming any other factor impairment that the patient may have. While a FVIII inhibitor ELISA is available, as mentioned above, this assay is not a functional test.

Accordingly, because the testing approaches described above require the addition of exogenous factors via the addition of normal plasma or, in some cases, the simultaneous addition of multiple purified exogenous factors, such testing approaches do not capture the patient's endogenous clotting phenotype. Further, all of the testing approaches currently require platelet-poor plasma and therefore do not account for the role of platelets, red blood cells, and white blood cells in the clotting time and clotting phenotype. Accurate monitoring of coagulation phenotype is important for making treatment and dosing decisions for patients, especially for patients suffering conditions that result in acute or transient coagulopathies, such as trauma or infection. For example, if a patient is determined to have FIX deficiency, but is in an acute inflammatory state where levels and therefore activity of FVIII and FI are increased, the addition of exogenous FIX (for the treatment of the deficiency) may lead to a faster clotting time than it would in a patient in a non-inflammatory state. In addition, if a patient is deficient in fibrinogen due to a consumptive coagulopathy, such fibrinogen deficiency potentially would be missed with the addition of normal plasma to the patient plasma, as the normal plasma would provide fibrinogen and thereby mask the fibrinogen deficiency in the patient's plasma.

Thus there is a need in the clinic for coagulation tests that can detect, characterize, and/or quantify factor-specific impairments in coagulation to better manage patients at high risk of severe bleeding or clotting, including, but not limited to, patients with factor deficiency and/or factor inhibition (e.g., patients with anti-factor auto-antibodies and/or who have taken drug inhibitors). Additionally, there is a need for tests that assess the degree to which a factor deficiency or inhibition affects clotting time, and that further account for a patient's endogenous coagulation profile, such that treatment decisions are based on all factors contributing to a patient's coagulation phenotype rather than on only one or two specific factors.

### SUMMARY OF THE INVENTION

Embodiments of the present invention provide methods and devices for evaluating coagulation, including methods and devices for detecting an impairment in coagulation factor function, such as impairments caused by coagulation factor deficiency and/or inhibition, or that are due to resistance to natural anticoagulants. An impairment in coagulation factor function may result in abnormal or impaired clot formation (e.g., thrombosis), or in abnormal or impaired clot degradation (e.g., fibrinolysis). In various embodiments, the methods and devices of the invention measure coagulation of a sample in response to a gradient of one or more coagulation factors. These coagulation measurements can be evaluated to accurately profile coagulation factor impairments, including the presence of a factor deficiency or inhibition, or of resistance to anticoagulants, of the sample. In various embodiments, the invention provides point-of-care or bedside testing with a convenient, microfluidic device that can be used by minimally trained personnel.

In some aspects, the present invention provides methods for assessing coagulation or coagulation factor function in a sample of bodily fluid (e.g., a blood sample, or a sample of cerebral spinal fluid (CSF), peritoneal fluid, thoracic fluid, pleural fluid, or pericardial fluid). The method comprises adding a coagulation factor to two or more portions (e.g., aliquots) of the sample, each portion receiving the coagulation factor at a different concentration, and measuring clot formation (e.g., clot formation times) in response to the different factor concentrations. By assessing coagulation in response to the different concentrations of one or more coagulation factors, blood clotting function, including the presence and impact of Hemophilia A, B, or C or other coagulopathies, can be accurately profiled. In some embodiments, the presence or absence of a congenital and/or hereditary clotting abnormality is determined. In other embodiments, the presence or absence of an acquired clotting abnormality (e.g., anti-factor auto-antibodies or iatrogenic inhibition) is determined. In certain embodiments, changes in the concentration, activity, or inhibition of endogenous coagulation factors (including co-factors) in response to injury, infection, or inflammation (as some examples), and the effects of such changes on coagulation may be detected using the devices and methods described herein. The methods of the present invention may be performed using a microfluidic device as described herein, where one or more of the channels can be configured to trigger formation and/or localization of a clot.

The methods and devices described herein permit evaluation of the effect of the specific factor in question in a targeted fashion, while also allowing for the other coagulation factors present in a patient sample to affect clotting time. This approach provides the ability to not only detect a specific factor deficiency or inhibitor, but also to evaluate how the addition of that factor affects clotting time in a concentration-dependent fashion. For example, if a patient has decreased Factor VIII levels but increased Factor XI levels, this approach would identify the decrease in Factor VIII, as long as it results in a prolonged clotting time; and if the patient's increased Factor XI level resulted in a relative shortening of the clotting time, as Factor XIa above certain concentrations has been shown to bypass Factor VIII (see Kluft et al., Thrombosis Res. 135:198-204 (2015)), the testing approach described herein would take this Factor XI effect into account. This type of information would permit a precision-medicine approach to coagulation management, guiding clinicians to potentially giving lower levels of replacement Factor VIII to patients with very high Factor XI levels in order to avoid pushing them toward a pro-thrombotic state. This type of coagulation management is not possible using the traditional factor deficiency and inhibitor assays described above because, in an attempt to be so specific for the factor in question, the contributions of the other endogenous coagulation factors to the patient's clotting phenotype are masked by the plasma adsorption treatment and/or the addition of multiple exogenous coagulation factors in the form of purified factor mixtures or normal plasma. In other words, a clinician using the coagulation assays currently available may not be able to determine how a patient's other endogenous coagulation factors (factors other than the factor being tested) affect the patient's clotting time, as the assays require the addition of exogenous factors that may overcome any other factor deficiencies and would mask any compensatory changes in factor levels.

There are also situations where currently available tests may result in false positive identification of factor deficiency or inhibitor presence, such as when there is anticoagulant contamination in the sample. For example, Factor Xa inhibitors may result in a dose-dependent prolongation of the PT and/or aPTT as well as a decrease in Factor Xa chromogenic activity. Factor IIa inhibitors may also result in a dose-dependent prolongation of the PT and/or aPTT. Anticoagulant contamination can be due to patient drug administration or due to intravenous or intra-arterial catheter contamination. In addition, as discussed above, in the aPTT-based testing approaches the presence of lupus anticoagulant in some circumstances (depending on the particular aPTT reagent used) could also result in a false-positive result for inhibitors or deficiency.

In embodiments of the novel coagulation testing approach described herein, one or more coagulation factors upstream, and one or more coagulation factors downstream of the factor in question are each added to their own respective portions of the patient's sample (e.g., blood sample). The methods and devices described herein allow clinicians to identify other causes of prolongation of clotting time, aside from causes due to a deficiency or inhibition of the factor in question, and thereby provide a higher level of specificity by better pinpointing where in the entire cascade there may be a deficiency or an inhibitor acts. For example, the addition of Factor Xa as the downstream coagulation factor (in a test for FVIII impairment, for example) can serve as a positive control, such that the presence of any Factor Xa inhibitor would be detected, and likewise any cause of prolongation of clotting time at or downstream of Factor Xa, such as afibrinogenemia, would be detected. The addition of Factor IIa could serve as another downstream positive control, and although Factor IIa addition would identify the presence of thrombin inhibitors and certain heparins, it would miss specific Factor Xa inhibitors (as FIIa acts downstream of FXa). However, adding Factor Xa to one portion of the sample and adding Factor IIa to another, separate portion of the sample at appropriate concentrations allows for the identification of both Factor Xa and Factor IIa anticoagulants and provides a more comprehensive positive control. Additionally, as long as specific phospholipid reagents are not used as the clotting activator in this assay (e.g., a contact activator that does not interact with antiphospholipid antibodies is instead used), the presence of lupus anticoagulant in the sample would not result in a false positive identification of factor inhibition or deficiencies.

Further, because the current traditional testing protocols involve the addition of normal plasma and, in some cases, also require depleting endogenous factors from the patient's plasma sample, these approaches are unable to differentiate between inhibitors to the inactive (e.g., precursor) form of the factor and inhibitors to the activated form of the factor. For example, while a patient's sample may have inhibitors targeting Factor IXa, these current testing approaches will be able to assess only that there is an inhibitor to Factor IX/IXa, without assessing whether the inhibitor may be specific to FIX and not to FIXa or vice versa. In contrast, the methods of the present invention can differentiate between these two classes of inhibitors, as the methods involve adding the inactive or the active form of specific factors to separate portions of a sample. Determining whether inhibitors target inactive versus active factors would be useful for pre-clinical and clinical drug development, as well as for the study of spontaneous inhibitor development (e.g., anti-factor auto-antibody development).

The methods and systems described herein apply an upstream-downstream logic-based approach to coagulation phenotype evaluation where the addition of specific factor(s) allows for the identification of coagulation factor deficiency and/or the presence of inhibitors, including factor-specific drug inhibitors and/or anti-factor auto-antibodies. Drugs here include both natural and synthetic drugs, peptides, proteins, aptamers, and antibodies (whole immunoglobulins or fragments thereof). For example, using the methods described herein, a clinician can detect and differentiate between the presence of Factor IIa inhibitors and Factor Xa inhibitors, as can occur via multiple anticoagulant/antithrombotic drugs. The methods and systems described herein can be customized for the identification, screening, and monitoring of various types of drugs, including, but not limited to, Factor XI inhibitors, Factor XIa inhibitors, Factor XII inhibitors, and Factor XIIa inhibitors. For example, this testing methodology can be used to identify, differentiate among, and quantify the antithrombotic effects of FXa, FIIa, and FXI and/or FXIa inhibitors in one comprehensive assay that tests the effects of factors that act upstream, downstream, and at each point of potential inhibition. See, for example, FIGS. 32-34. Inhibitors may also be used for the manipulation of the anticoagulation or fibrinolysis arms of the coagulation pathway; such manipulation could include, for example, Tissue Factor Pathway Inhibitor (TFPI) inhibition, or Tissue Plasminogen Activator (tPA) or Activated Protein C (APC) administration. The assay approach and methods described herein can also be used to evaluate FXa, FIIa, and FXI and/or FXIa inhibitors in conjunction with, e.g., tPA activity. Additionally, due to the use of inactivated form or activated form of the coagulation factor that is added, the methods described herein are able to identify inhibitors that are specific for only one form (active or inactive) and not the other—e.g., the devices and methods described herein can identify inhibitors that target Factor XI (and that do not also target Factor XIa) and can identify inhibitors that target Factor XIa (and that do not also target Factor XI). Such differentiation cannot be accomplished with the currently available methods described above.

As used herein, unless described otherwise, a “blood sample” refers to a whole blood sample or a plasma sample. The term plasma includes both platelet-rich-plasma (PRP) and platelet-poor-plasma (PPP).

The term “coagulation factor” as used herein means any factor (including any co-factor) implicated in the coagulation cascade (intrinsic, extrinsic, and common pathways), including, e.g., Factors I to XIII, von Willebrand factor, prekallikrein (Fletcher factor), high-molecular-weight kininogen (HMWK) (Fitzgerald factor), fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2), Tissue Factor Pathway Inhibitor (TFPI), and cancer procoagulant. A coagulation factor can be in activated form or inactivated (e.g., precursor) form. For example, the difference in coagulation in response to the addition of the activated versus inactivated form of a coagulation factor can help pinpoint the exact deficiency or inhibition present in the sample. In other embodiments, e.g., for detection of a genetic clotting abnormality, the coagulation factor may be added in its inactivated form (e.g., Factor VIII, Factor IX, or Factor XI). Further, a coagulation factor can be from a human, from an animal (such as cow, pig, or other), or can be a synthesized or recombinant protein.

By measuring clot formation (e.g., clot formation times) in response to increasing concentrations of exogenously added coagulation factors, the presence and/or point of deficiency and/or inhibition (such as by an anti-factor antibody or a drug inhibitor) can be determined. For example, a sample that has a coagulation factor deficiency will show a decrease in clotting time as the deficient coagulation factor is added to the sample. When the activated form of the deficient coagulation factor is added, the clotting time should decrease to achieve a clotting time that is normal because the point of deficiency (the inactivated form of the coagulation factor) has been bypassed. Meanwhile, when a coagulation factor (activated or inactivated) upstream from the point of deficiency is added (in increasing amounts), the clotting time should remain prolonged (relative to a normal clotting time). These clotting times are compared to the clotting time when a coagulation factor downstream to the point of deficiency is added; when the inactivated form of the downstream coagulation factor is added, clotting time does not achieve a normal clotting time, whereas when the activated form of the downstream coagulation factor is added, the clotting time decreases to a clotting time within a normal range. See, e.g., FIGS. 9-14, 25, 29, 32, 34.

Furthermore, by evaluating clot formation (e.g., clot time) in response to the addition of coagulation factors as described herein, factor deficiency can be differentiated from factor inhibition. In the case of factor deficiency, the addition of physiological levels of the deficient coagulation factor decrease clotting time and result in a normalization in clotting time. In the case of factor inhibition (e.g., by an anti-factor auto-antibody), depending on the concentration and activity level of the factor inhibitor, a supra-physiological level of the factor will be necessary to decrease the clotting time and reach a clotting time within a normal clot time range. Additionally, the level of factor inhibition can be determined by evaluating the concentration-dependent response in clotting time. For example, a high level of anti-factor autoantibodies and inhibitory activity will result in a different clotting time response (clotting curve) to various concentrations of exogenously added factor, due to the high level of factor required to shorten clotting time and result in a normalization of the clotting time compared to the factor levels required to shorten and normalize the clotting time in a sample with a moderate level of anti-factor autoantibodies and inhibitory activity. See, for example, FIGS. 26-28, 33. In the case of a mild level of inhibitor, inhibition may not be able to be differentiated from factor deficiency, depending on the inhibitor concentration and inhibition activity level.

As used herein and unless otherwise specified, a physiological level of a factor may refer to the level or concentration of that factor that is normally present in the species (e.g., human); such level or concentration can be represented as a range (see, e.g., FIG. 35). The minimum level of a factor required for clot formation in the methods and devices described herein may be less than the level at which the factor is normally present in the species. For example, while fibrinogen is normally present at a level of 150-400 mg/dL in human subjects, clot formation can be observed in samples having fibrinogen at a level as low as about 50 mg/dL or so. In certain embodiments, references to physiological factor levels includes such minimum levels required for clot formation.

The methods and devices described herein can thus be used to identify, differentiate among, and quantify the effects of multiple coagulation factors simultaneously by providing a matrix wherein multiple coagulation factors are added in a strategic upstream-downstream logic-based approach, each factor added to a separate portion of a sample, to pin-point the exact coagulation factor deficiency or inhibition in the sample with one single test. For example, a clinician could identify the presence of, and differentiate the effects of FIIa inhibitors, FXa inhibitors, FXI inhibitors, and FXIa inhibitors using one single assay. See, for example, FIGS. 32-34. A clinician or other health-care worker could also use this approach to create a complete hemophilia screening panel, identifying Factor VIII, Factor IX, and Factor XI deficiency or inhibition in one single device, or in a single test where different factors are added to different portions of a patient's sample (e.g., sample of whole blood or of plasma). See, for example, FIG. 14 and FIG. 25. Additionally, use of a downstream, positive control coagulation factor (such as Factor Xa and/or Factor IIa) can detect the presence of contaminating or residual anticoagulation agents, which could impede the hemophilia testing. As an example, if a patient has residual heparin contamination from his/her blood sampling port, such contamination would be detected with the Factor Xa and/or Factor IIa coagulation testing lane; in this case, hepzyme could be added to neutralize the heparin, allowing for the more accurate screening of the hemophilia panel. In some embodiments, the test could include hepzyme as a pre-treatment to the blood sample, or hepzyme can be pre-loaded in the device itself.

In some embodiments, results for a patient sample can be compared to one or more reference standards. Such reference standards include reference standards for normal clotting, reference standards abnormal clotting, reference standards corresponding to anticoagulant therapy with particular agents, and reference standards based on patient samples that have the same coagulation impairment that is being tested for. In some embodiments, reference standards are personalized for a patient and can be used for monitoring the patient's response to therapy, such as with longitudinal testing.

In various embodiments, clotting curves can be constructed to characterize the response of clot formation to the addition of various coagulation factors in increasing concentrations or amounts. These clotting curves allow for the identification of, and quantification of the amount of coagulation inhibitors present in the sample, to thereby guide patient care. The change in the clotting times, and therefore in the clotting curve, after the administration of any treatment can be used to assess the patient's coagulation response to the treatment. The change in the clotting curve can also be used to monitor long-term patient treatment, such as for patients being administered replacement coagulation factors or for patients receiving a gene therapy (or other long-term therapy) that alters their coagulation phenotype.

In some aspects, the invention provides a microfluidic device for evaluating coagulation in a sample. The device may include a series of channels in a substrate, each channel having an area with a geometry to trigger and/or localize formation of a clot, to allow for evaluation of clot formation in response to one or more reagents, such as a coagulation factor added at a specific amount or concentration. The channels in the series may all have the same geometry, so as to provide identical clot formation environments and thereby trigger the same clot formation properties (when exposed to the same sample and reagents). By evaluating clot formation in the presence of a gradient of one or more coagulation factors, such a device allows for sensitive and specific detection of coagulation impairments, including coagulation factor deficiency or inhibition. While various embodiments are discussed with reference to “channels,” other terms (such as, e.g., lanes, compartments, partitions) can describe spaces that are physically separated from each other, and that in some embodiments have the same geometry as each other.

In aspects of the invention, the microfluidic device for evaluating coagulation includes a plurality of channels formed in a substrate, each channel including a clot forming area having a geometry configured to trigger and/or localize formation of a clot. In some embodiments, the clot forming areas of the plurality of channels are arranged in a central region of the substrate, such that clotting can be simultaneously imaged or analyzed across the channels. E.g., see FIGS. 1A-1B, 2B. The device may further include a plurality of sample input ports to receive a sample (e.g., whole blood or plasma, CSF, or other bodily fluid), each sample input port connected to a first end of one of the plurality of channels. See, e.g., FIGS. 1A-1D. In other embodiments, the device has a single sample input port in fluid communication with each of the plurality of channels. E.g., see FIG. 5A. In some embodiments, each channel has an independent output port, and each channel's output port is connected to a second end of one of the other channels. In embodiments where each channel has its own sample input port and its own output port, the input and output ports can be arranged in an alternating pattern at a periphery of the substrate. See, e.g., FIGS. 1A-1B, 2A. In alternative embodiments, the input and output ports are arranged in a pattern other than an alternating pattern.

The term “central region” as used herein means a region that is located in the center of a substrate relative to a periphery of the substrate and can include a region that is positioned off-center. For example, depending upon the configuration, the central region might be off-center and clot formation in the areas in the channels in which clot formation begins can be controlled by the flow patterns in the channels.

In some embodiments, the clot forming areas in each of the plurality of channels are arranged in a region of the substrate that is not central, such as, for example, the periphery of the substrate. E.g., see FIGS. 5A-5B.

Each channel may further comprise one or more additional input ports to receive reagents, such as coagulation factor(s) and/or calcium. In some embodiments, there is more than one input port (e.g., for introducing sample and one or more reagents) per output port. For example, in certain embodiments, there can be one input port for the sample and one or two input ports for the reagents (e.g., coagulation factor and, optionally, calcium). E.g., see FIG. 1B. In some embodiments, there is one common input port to receive the sample, and each channel comprises its own additional input ports (e.g., 1 or 2 input ports) for reagents.

In some embodiments of the microfluidic device, each clot forming area can be configured to create an area of stasis or an area of disruption in fluid flow to trigger and/or localize formation of a clot. In some embodiments, each clot forming area can be configured to create an area of flow disturbance to trigger and/or localize clot formation.

Channels of the microfluidic device can be coated with, contain, or otherwise include a coagulation factor at different amounts or concentrations. For example, a first group or series of the plurality of channels can be coated with, contain, or otherwise include a first coagulation factor (with each channel in the first group or series including the first coagulation factor at a concentration that is different from each of the concentrations in the other channels of the group or series), and a second group or series of the plurality of channels can be coated with, contain, or otherwise include a second coagulation factor (with each channel in the second group or series including the second coagulation factor at a concentration that is different from each of the concentrations in the other channels of the group or series). Further, in some embodiments, one or more of the plurality of channels is a negative control channel, e.g., the channel is not coated with and otherwise does not include a coagulation factor. In other embodiments, the device does not comprise such a negative control channel. In some embodiments, there may be more than one coagulation factor in each channel. In other embodiments, the device may comprise multiple channels, each channel having a different coagulation factor, but all factors are included at a single (and in some instances, the same) concentration.

Coagulation factor(s) that may be included in one or more channels may be in suspension or solution, may be surface-bound (such as by dry-spotting), or may be lyophilized and not surface-bound. The coagulation factor(s) can be pre-included in the channel(s) (e.g., at the time of manufacturing the device), can be added prior to placing the sample into the device, or can be added into the device through an input port (or through multiple input ports) concurrently with the sample or after the sample has been added.

In embodiments of the microfluidic device that include first and second groups of channels (whether or not such embodiments may also include a negative control channel in addition to the first and second groups of channels), each channel in the first group of channels can be coated with, contain, or otherwise include a first coagulation factor at a unique amount or concentration, and each channel in the second group of channels can be coated with, contain, or otherwise include a second coagulation factor at a unique amount or concentration. In some embodiments, the two groups of channels may contain the same coagulation factor, but one group includes the inactivated form of the factor and the second group includes the activated form of the factor. In some embodiments, the microfluidic device may contain more than two groups or series of channels, such as three, four, five, or more groups, wherein each group or series of channels is coated with, contains, or otherwise includes a different coagulation factor at an increasing concentration across the channels in the group or series (e.g., a microfluidic device may contain four groups of channels, each group having channels coated with, containing, or otherwise including a coagulation factor selected from Factors XI, XIa, VIII, VIIIa, IX, IXa, Xa and/or IIa, wherein no two groups include the same factor, and with respect to each group's channels, the channels in a group contain the factor at increasing concentrations (a concentration gradient); Factor VIII may or may not include Factor VIII in the complexed form with von Willebrand Factor (vWF)). By measuring clot formation (e.g., clotting time) as a function of coagulation factor concentration gradients, the sample's clotting properties can be profiled at several specific points of the coagulation pathway(s), providing a clinician with detailed and specific information concerning the patient's clotting physiology and/or the status of any therapeutic intervention.

For some of the embodiments with two or more groups of channels as described above, the second coagulation factor can be upstream in the coagulation cascade from the first coagulation factor. In addition, the first coagulation factor can be in active or inactive form, and the second coagulation factor can be in active or inactive form. In certain embodiments, the downstream factor is in active form.

The microfluidic device may further include a detection device configured to measure clot formation times in each of the channels. The microfluidic device may further report the clot formation times measured, to assess coagulation based on the clot formation times. For example, the detection device can be configured to simultaneously image the clot forming areas to measure clot formation times. In some embodiments, the degree of clot formation in each of the channels is quantified at a fixed time or at fixed times. For example, the detection device in connection with the methods and devices described herein can include a microscope and an image sensor. Imaging the clot forming areas can include bright-field imaging. For the devices and assays described herein, clotting times can also be measured with other methodologies, such as methodologies based on light absorbance, fluorescence measurements, ultrasound, etc., and the detection device can be configured to employ one or more of these other methodologies. Ways to detect clotting also include, but are not limited to: detection based on electrical impedance, electrical capacitance, or electrical resistance; detection based on changes in flow dynamics, such as by measuring flow velocity (e.g., by the addition of beads (e.g., magnetic beads) and quantifying bead flow rate and/or number of moving beads), and detection based on changes in viscoelasticity; detection based on changes in pressure at and/or around the site of clot formation; detection based on rheological assessments; fluorescence detection (such as with fluorescent fibrinogen); detection based on changes in turbidity; infrared light detection; infrared spectroscopy; detection using acoustic and/or photonic sensors; detection based on flow cytometry; and visual clotting detection.

In some embodiments, the methods of the invention do not employ a microfluidic device and instead use wells or containers suitable for inducing and measuring formation of a clot.

In addition to clot formation times, other characteristics of clot formation can be assessed. For example, properties of the clot such as size, strength, density, and composition can be analyzed, in addition to the time to form, retract, or dissolve a clot. Such properties may be assessed using the same modality that is used to detect clot formation times or may be assessed using a different detection modality. A qualitative measure of clot formation, in addition to clot formation times, can be useful, e.g., to determine the most sensitive detection mode for coagulation.

In some embodiments, clot lysis (which may also be called fibrinolysis or clot degradation) can be assessed in addition to clot formation. For example, if a patient is on a fibrinolytic or thrombolytic agent, a clinician may wish to evaluate the clot when it is being formed as well as evaluate its breakdown over time. In certain embodiments, the same methods described herein and known in the art to detect clot formation can be used to assess clot lysis over time. Assessing clot lysis may be useful in cases where there are multiple derangements to the coagulation system, where a treatment is administered, or where there are multiple targets or effects of the inhibitors detected. The methodology used for evaluating fibrinolysis may be the same as or different from the methodology used for clot detection (e.g., for measuring clotting time). For example, viscoelastometry is able to evaluate fibrinolysis, independent of clotting time.

Regarding the use of viscoelastic testing, such method can be used to evaluate both clot formation and fibrinolysis. Evaluating both processes would be useful for detecting clotting abnormalities in patients that are hypocoagulable due to problems with fibrinolysis or due to iatrogenic administration of fibrinolytic and thrombolytic drugs. See, for example, C. Mauffrey, et al., Bone Joint J. 96-B:1143-54 (2014).

For any of the devices and methods described herein, the sample may be any bodily fluid sample in which coagulation may occur, or that may or is known to contain coagulation factors. Such bodily fluids include blood (including, e.g., whole blood or plasma (which can be platelet-rich-plasma (PRP) or platelet-poor-plasma (PPP)); accordingly, a blood sample can be a whole blood sample or a plasma sample. Using whole blood can be particularly beneficial or convenient for certain applications, such as those implemented at the bedside of a patient or when a complete assessment of physiologic coagulation status is required (such complete assessment would include platelets, red blood cells, and white blood cells in the evaluation). Other bodily fluids that can be used in the devices and methods described herein include, for example, cerebral spinal fluid (CSF), amniotic fluid, peritoneal fluid, pericardial fluid, and pleural fluid.

The devices and methods can be applied to all individuals, including mammals (e.g., humans, such as human patients, as well as non-human mammals), reptiles, birds, and fish, among others, and can be useful for research and veterinary medicine. An individual can be, for example, mature (e.g., adult) or immature (e.g., child, infant, neonate, or pre-term infant).

The devices and methods described herein can be used for diagnostic purposes as well as for research and discovery into the coagulation cascade. For example, the devices and methods described herein can be useful for basic drug discovery, for understanding disease or disorder pathophysiology, for example, in the context of infectious diseases that result in pathological bleeding or clotting (SARS-CoV-2 virus, Dengue virus, Zika virus, Ebola virus, etc.), and for clinical and pre-clinical assessment of new drugs/compounds/treatments.

In addition, the devices and methods described herein can be used to guide therapy of a patient. For example, the results of a factor deficiency identification as described herein can be used to determine optimal therapy, and the assays and devices may be further used to monitor response to treatment. Similarly, physicians can use the results of a factor inhibitor identification to guide their selection of appropriate treatment and to monitor the patient's response to that treatment. The methods and devices described herein can also be used to monitor a patient for the development of, or increase/decrease in the activity of, factor inhibitors. The detection and identification of factor inhibitor development can be used in the emergency setting, as well as for the monitoring of new therapies such as gene therapies. The devices and methods of the present invention can also be used for the rapid screening of the cause of coagulopathies, such as in the testing of an infant with spontaneous bruising or prior to an invasive procedure, such as emergency surgery or circumcision. In some cases, for patients who receive factor-replacement only at specific times, such as prior to an invasive procedure or high-risk injury activity, the methods and devices provided herein can be used to obtain a pre-factor replacement clotting time and post-factor clotting time to confirm therapeutic levels are achieved.

The devices and methods also can be used to detect and evaluate response to treatment of various coagulation factor inhibitors. For example, the devices and methods can be used to detect the presence of, and to differentiate between Factor Xa inhibitors, Factor IIa inhibitors, Factor XI inhibitors, and Factor XIa inhibitors. Such assessment could be important as new anticoagulants and antithrombotic drugs are developed and the need to identify and quantify the effects of these drugs in emergency settings grows increasingly critical and complex. For example, if a clinician does not have access to a patient's medical record and the patient is suspected to be on an anticoagulant or antithrombotic agent, the clinician could use a factor inhibitor screening test as provided herein to detect whether the patient is on an anticoagulant/antithrombotic drug, identify the class of the inhibitor (i.e., which coagulation factor is inhibited), and quantify the effect of the inhibitor on the patient's clotting time. This same test can be used to monitor patient response to therapy (e.g., if a patient is receiving a reversal or bypassing agent, or an anticoagulant). For example, the devices and methods described herein can be used to detect response to bypassing agent therapy, such as in the case of administration of FVIIa to a patient anticoagulated with a FXa inhibitor, or to monitor a patient receiving a reversal agent, such as a patient receiving protamine to reverse the anticoagulant effects of heparin. As an additional example, the devices and methods described herein can be used to detect resistance to an anticoagulation treatment, such as in the case of a patient with low antithrombin III activity, which can result in reduced anticoagulant activity of antithrombin III-dependent drugs, such as heparin.

Other aspects and embodiments of the invention will be apparent from the following detailed description.

### BRIEF DESCRIPTION OF THE DRAWINGS

The patent or application file contains at least one drawing or photograph executed in color. Copies of this patent or patent application publication with color drawings or photograms will be provided by the Office upon request and payment of the necessary fee.

FIGS. 1A-1D are schematic illustrations of microfluidic device layouts employing multiple sample ports according to example embodiments of the invention.

FIG. 2A is a top view of the circular microfluidic clotting device according to an example embodiment.

FIG. 2B is a magnified view of the central portion of the device of FIG. 2A. FIG. 2B illustrates exemplary geometries of clot formation areas.

FIGS. 3A-3C illustrate clot detection using plasma and fluorescent-labeled fibrinogen within a microfluidic device having four channels according to an example embodiment. FIG. 3A is a top view bright-field image of a central portion of the example microfluidic device. FIG. 3B is a fluorescent image of clot formation using the device of FIG. 3A. FIG. 3C is a fluorescent image showing a magnified view of a clot forming area.

FIGS. 4A and 4B are bright-field images illustrating clot detection using whole blood in a parallel microfluidic channel device employing a FXa gradient, according to an example embodiment. FIG. 4A contains no anticoagulant. FIG. 4B contains unfractionated heparin.

FIGS. 5A and 5B are schematic illustrations of microfluidic device configurations employing a single port for sample input according to example embodiments of the invention.

FIG. 6 is a flow diagram of an assay or a method according to example embodiments of the invention.

FIG. 7A is a graph of example data illustrating detection of Rivaroxaban, using a FXa gradient.

FIG. 7B is a graph of example data illustrating detection of Apixaban, using a FXa gradient.

FIG. 7C is a graph of example data illustrating detection of Edoxaban, using a FXa gradient.

FIG. 7D is a graph of example data illustrating detection of Dabigatran, using a FIIa gradient.

FIG. 8 is a diagram illustrating a basic clotting cascade.

FIG. 9 is a diagram illustrating how to detect FXa impairment by employing coagulation factor gradients.

FIG. 10 is a diagram illustrating how to detect FIIa impairment by employing coagulation factor gradients.

FIG. 11 is a diagram illustrating how to detect and differentiate between FIIa and FXa inhibition in a sample by employing coagulation factor gradients.

FIG. 12 is a diagram illustrating how to detect indirect FXa impairment by employing coagulation factor gradients.

FIG. 13 is a diagram illustrating how to detect and differentiate between FXIIa and FXIa inhibition in a sample.

FIG. 14 is a diagram illustrating how to detect and differentiate between various types of hemophilia by employing coagulation factor gradients.

FIG. 15 is a diagram illustrating how to detect impairments with fibrinogen or FXIII (e.g., FXIII deficiency) by employing coagulation factor gradients.

FIGS. 16A-16C illustrate Clotting Curve Scores (CCS) for FXa and FIIa inhibitors at various concentrations.

FIG. 17 shows Table 1 of patient descriptive statistics (Example 17).

FIGS. 18A-18C illustrate measurements of sensitivity and specificity of prothrombin time (PT) (FIG. 18A) and international normalized ratio (INR) (FIG. 18B) for FXa inhibitor (FXa-I) anticoagulation.

FIGS. 19A-19G illustrate example clotting time data and comparative clotting curves.

FIGS. 20A-20E illustrate Clotting Curve Score (CCS) analysis and evaluation of CCS utilization for the detection of FXa-I in patient samples.

FIGS. 21A and 21B illustrate example functional drug concentration calculation.

FIG. 22 illustrates a current decision-making paradigm for a patient that is bleeding or at high risk.

FIG. 23 illustrates a proposed improved decision-making paradigm using embodiment(s) of the present invention for a patient that is bleeding or at high risk.

FIGS. 24A and 24B illustrate detection of decrease in FXa inhibition by a FXa-inhibitor after the addition of activated prothrombin complex concentrate (aPCC).

FIG. 25 is a table providing representative clotting times for various plasma samples in the presence of exogenous coagulation factors, including both the inactive and active forms of specific factors; the clotting times illustrate how embodiments of the invention can be used to detect and differentiate among Hemophilia A, B, and C.

FIG. 26 is a clotting curve showing the change in clotting time when various concentrations of Factor IX are added to Factor IX-deficient plasma or to plasma containing anti-Factor IX antibodies, and illustrating how embodiments of the invention can be used to differentiate between factor deficiency and factor inhibition.

FIG. 27 is a schematic illustration of how clotting curves are used to distinguish between a factor-deficient and a factor-inhibitor sample, using Factor IX as an example.

FIGS. 28A-C are representative clotting curves demonstrating the change in clotting times as exogenous coagulation factors are added to Hemophilia C (FXI-deficient) and FXI-inhibitor samples (FIG. 28A), Hemophilia A (FVIII-deficient) and FVIII-inhibitor samples (FIG. 28B) and Hemophilia B (FIX-deficient) and FIX-inhibitor samples (FIG. 28C). FIG. 28D is a table providing descriptive information on the plasmas of FIGS. 28A-C, including factor activity levels and factor inhibitor levels.

FIG. 29 is a diagram illustrating how to detect Factor V Leiden disease by employing coagulation factor gradients.

FIG. 30 is a diagram illustrating how to detect hyperfibrinolysis by employing coagulation factor gradients.

FIG. 31 is a table providing representative clotting times that indicate an impairment in fibrinogen.

FIG. 32 is a diagram illustrating how to detect and differentiate among Factor IIa, Factor Xa, Factor XI, and Factor XIa inhibition.

FIGS. 33A-C show embodiments of assays used to detect the presence and level of factor inhibition.

FIG. 34 is a diagram illustrating how to detect and differentiate among Factor IIa, Factor Xa, Factor XI, Factor XIa, Factor XII, and Factor XIIa inhibition.

FIG. 35 is a table providing representative factor levels as reported in the literature.

### DETAILED DESCRIPTION

The invention generally relates to methods and devices for the detection of coagulation factor deficiency and/or inhibition.

Congenital and/or hereditary and acquired coagulopathies are commonly complex and time-consuming to diagnose. Deficiencies (which may result from decreased factor level, and/or from factor abnormalities that decrease function, even if level is not decreased) may present during childhood or upon the first surgery or injury of the affected individual when bleeding times are prolonged. The diagnosis of a factor deficiency usually requires specialty training, referral to a specialist, and multiple sequential complex coagulation tests. Acquired coagulopathies, such as the development of anti-factor auto-antibodies, require the same level of specialty involvement and testing, although these cases commonly present as severe, life-threatening bleeding events in an acute setting. Currently, rapid (e.g., <30 minute turn-around time), point-of-care tests for the identification of and differentiation between factor deficiency (whether a result of decreased factor level or of an abnormality that decreases factor function) and factor inhibition are not available. Having a test available that not only can identify which factor is impaired, but that can also differentiate between deficiency and inhibition and quantify the impairment's effect on clotting time, would provide physicians with easily accessible, interpretable, and actionable information in treatment decision-making and monitoring.

For patients that have factor inhibitors, such as anti-factor antibodies, clinical presentation is usually adverse, uncontrolled bleeding events. In these cases, providing the positive identification of the presence of a factor inhibitor would provide valuable functional clinical information to the doctor to help personalize the treatment and dosage, and would be of great benefit to the patient and possibly decrease subsequent, related adverse events, such as in the case where a patient with hereditary Hemophilia A presents with Acquired Hemophilia A in adulthood and is treated with Factor VIII and yet continues to have bleeding. The information provided by the devices and methods described herein would allow the clinician to determine that the patient's bleeding is inhibitor-dependent bleeding, and would allow the clinician to provide the correct amount of replacement factor or provide another bypassing reagent to aid in hemostasis. Embodiments of the invention also provide clotting panels that evaluate the coagulation, fibrinolysis, and platelet function within an individual. The microfluidic technology and advanced assays described herein in some embodiments further provide custom clotting panels, whereby clinicians can determine a patient's coagulation function bedside. These embodiments provide for vast improvements in patient care, including in the urgent care setting.

While the devices and methods of the invention provide rapid and easy-to-interpret assays, they also provide customizable devices and methods, allowing for the selection of clinically-relevant coagulation and platelet function testing for each customer and/or end-user segment. Because embodiments of the devices and methods can be used in a bedside platform, they can also be utilized for trend-monitoring in patients on various treatments (including at the hospital, at coagulation clinics, and at home). For example, in certain aspects of the invention, a concentration gradient of a factor is added to a group of channels (or, e.g., to a group of wells or containers), each channel containing a portion of the sample, after the sample has been subdivided into and/or distributed among the channels in the multiple groups of channels; similarly, a concentration gradient of a second factor is added to the channels of a second group of channels. Such method permits evaluation of coagulation function/inhibition and identification and differentiation between various coagulation abnormalities within a sample. Such embodiments of the invention (e.g., clotting panels, assays, etc.) are useful for assessing coagulation in patients that have poor medical compliance, in patients whose medication dosage and/or time of medication administration are unknown, and in patients who are unconscious and the doctor, surgeon, or other healthcare provider must determine whether the patient has any coagulation impairment. Further, embodiments of the devices and methods described herein can help clinicians monitor patient treatment, such as factor replacement, gene therapy, or bypassing agents, and help guide the administration of various hemostatic treatments in the case of active bleeding.

Examples of potential users for products and services based on embodiments of the present invention can range from healthcare workers, e.g., clinicians and veterinarians, to researchers in pharmaceutical research and development.

Embodiments of the present invention can be used for patient care in various settings. In some embodiments, a patient is scheduled for surgery or is in need of an invasive procedure, and the methods and devices of the invention can be used for clinical decision-making, including for preparing the patient for the procedure to minimize bleeding risks. In additional embodiments, a patient is administered a drug that affects coagulation, and the methods and devices of the invention can be used for early evaluation of drug action and for selection of the appropriate therapy and dose. In further embodiments, a patient receives a drug or blood product, and methods and devices of the invention can be used to monitor and guide administration and dose. In other embodiments, a patient with a familial history of coagulation abnormalities or spontaneous bleeding/bruising is screened for the presence of coagulation factor deficiency (e.g., abnormality) and/or inhibition. In some embodiments, the patient is an infant or neonate, where only small volumes of blood are available for evaluating coagulation (including for monitoring anticoagulant therapy or for detecting a congenital coagulation abnormality). In some embodiments, the patient is pregnant, and the methods and devices allow for detecting a congenital coagulation abnormality, or for early diagnosis of a condition that results in a coagulation abnormality such as acquired Hemophilia A.

In some embodiments, the patient or subject is non-human (e.g., such as a dog, cat, or horse). In some embodiments, the patient is a non-human mammal. The cost-restrictions and limited blood volume associated with veterinary patients and laboratory animal research result in a large need for coagulation diagnostics that are easy-to-use, require only microliters of blood, and have lower overhead costs. The novel coagulation testing platforms (e.g., assay, microfluidic device, and/or combination thereof) described herein satisfy such need.

In some embodiments, a patient is receiving an anticoagulant therapy, such as a heparin (such as low molecular weight or unfractionated heparin) (heparin is an indirect inhibitor of Factor IIa and Factor Xa), a Direct Oral Anticoagulant (DOAC) inhibiting Factor Xa (such as XARELTO® (Rivaroxaban), ELIQUIS® (Apixaban), SAVAYSA® (Edoxaban), or BEVYXXA® (Betrixaban)) or inhibiting Factor IIa (such as PRADAXA® (Dabigatran)), or an injectable Factor IIa or Factor Xa inhibitor (such as fondaparinux, bivalirudin, or argotraban). In certain embodiments, a patient is undergoing therapy with a different factor-specific inhibitor, such as an inhibitor targeting Factor XI and/or Factor XIa, Factor XII and/or Factor XIIa, Factor V and/or Factor Va, or Factor VII and/or Factor VIIa. In some embodiments, a patient is undergoing coagulation therapy for the prevention of fibrinolysis, such as with an inhibitor or antibody against plasmin or plasminogen, or for the promotion of fibrinolysis, such as with tPA. In addition, a patient may receive natural or synthetic coagulation factors for the manipulation of the coagulation system; for example, a patient may receive Factor VIIa, (activated) prothrombin complex concentrates (PCC or aPCC), or anti-inhibitor coagulant complex (FEIBA®).

Anticoagulant drugs are commonly used in many medical settings, including emergency and critical care, surgery, cardiology, and cancer. Several new anticoagulants have been introduced, but currently there are no tests that can reliably determine if a patient is taking the most optimal dose. Similarly, there are no reliable tests to monitor a patient's response to reversal or bypassing therapy. Too much anticoagulation can cause life-threatening bleeding, and too little anticoagulation can lead to an increased risk of stroke and heart attacks. Additionally, the inappropriate administration of specific agents may predispose a patient towards development of pathological blood clots, such as in the case of the administration of FEIBA® in some hemophilia patients without inhibitors (see FEIBA® product labelling).

In some embodiments, a patient may have pre-existing coagulation factor deficiency (e.g., abnormality), such as in hemophilia, and the detection of anti-factor antibodies is necessary. In certain embodiments, the quantification of the effect of anti-factor antibodies on coagulation must be determined and monitored before and after therapeutic treatment. Embodiments of the invention can be used as or incorporated into a bedside test that can accurately monitor therapeutic treatment, such as factor replacement or bypassing agents, and improve the safety for patients undergoing such treatment. The devices described herein can be used, and the methods provided herein can be performed, with minimal training and in an easy-to-interpret format. For example, the methods can be performed using a device requiring less than about 1 mL, less than about 500 μL, less than about 100 μL, or less than about 50 μL, or about 5 μL or less (e.g., in some embodiments, about one drop) of fresh or citrated whole blood, with the results being available within 10 minutes of starting the method.

Coagulation factor deficiency is most commonly treated with factor replacement. Depending on the severity of the deficiency and the patient's propensity for spontaneous or uncontrolled bleeding, the patient may receive regular infusions of coagulation factors, or the patient may receive the factors prophylactically, such as in the case of a planned invasive procedure. New treatments for coagulation factor deficiencies include long-term treatments, such as gene therapy. Because these treatments are still new and experimental, the long-term monitoring of these patients during clinical trials to determine factor levels over time and to confirm the lack of development of anti-factor antibodies is required. While patients with coagulation factor inhibitors may be treated with factor replacement, if inhibitors are present and active above low levels (mild based on Bethesda Assay results), they may require treatment with bypassing agents, such as activated coagulation factor concentrates, that may result in a pro-thrombotic state if the dose is too high. The diagnosis and management of coagulopathies is often complex and requires precise tests in order to optimally manage patients in the chronic and acute setting.

In some embodiments, methods of the present invention allow clinicians to detect a coagulation deficiency in a blood sample, and to pinpoint where the deficiency occurs within the coagulation cascade. Such methods may comprise comparing the clot formation times measured for the sample, to coagulation factor-specific clot formation reference ranges, e.g., from individual(s) who do not suffer from a coagulation cascade abnormality. In some embodiments, the reference ranges can be established using the detection method on a normal subject or subjects, e.g., individuals who do not suffer from a coagulation abnormality. In other embodiments, the reference range can be established based on the same individual from whom the test blood sample(s) is obtained. For example, the reference range can be established prior to commencement of a medical treatment of an individual, and the test sample can be obtained from the same individual after the commencement of the treatment. A reference range can also be established using a sample obtained from a relative (e.g., parent, sibling, or offspring) of the individual from whom the test sample is obtained. The reference range(s) may be tailored to or dependent on a particular assay configuration, including microfluidic device configuration. For example, the clotting time of a patient's sample can be compared to the clotting time of a normal sample at the testing time, or to a previously-determined reference range clotting time (which can be a clotting time or clotting time range that represents normal clotting time, or that corresponds to an impairment in a specific coagulation factor or combination of factors). In some embodiments, the methods involve the establishment and/or verification of reference ranges.

In some embodiments, reference ranges may be established from clotting time standards, such as standards based on the clotting times of samples from individuals who do not suffer from a coagulation cascade impairment. In some embodiments, the reference ranges are established from samples spiked with known concentrations of various anticoagulants or from samples depleted of specific factors; such samples may be commercially available.

It should be understood that clot formation times can also be compared to reference ranges from individuals who do suffer from a coagulation impairment. For example, it is common with reference intervals to have a “normal” interval range for people who do not suffer from a deficiency and an “abnormal” interval range for people confirmed to have that deficiency. Sometimes, there is a gray zone in between the normal and abnormal zones, which is indicative that further in-depth testing is required on that patient sample for a definitive diagnosis.

In some embodiments, methods of the invention do not comprise comparison of a sample's clotting time to a reference range or standard clotting time. In certain embodiments, the methods provide internal controls by evaluating the clotting response when coagulation factors upstream and downstream of a suspected point of impairment in the coagulation pathway(s) are added to the sample.

Descriptions of example embodiments are provided herein.

Embodiments described herein provide rapid assays (e.g., <30 minutes, <20 minutes, <15 minutes, or <10 minutes in some embodiments) for the detection of anticoagulants and platelet inhibitors in whole blood or plasma and the assessment of patient coagulation status. The availability of these customizable coagulation panels meets an unmet need within various coagulation testing environments by providing rapid, bedside diagnostics and drug and other therapy monitoring capabilities.

In certain embodiments, the methods include an assay wherein a specific coagulation factor suspected of being impaired (e.g., deficient (such as by having an abnormality) or inhibited, or by being resistant to an anticoagulant) is added to a blood sample (e.g., a whole blood or plasma sample), in various concentrations or amounts. For example, the coagulation factor can be added to divided portions of the sample in amounts that vary from each other by a factor of 2 up to a factor of 100. In some embodiments, a coagulation factor is added to divided portions of the sample at concentrations increasing by a factor of 5 up to a factor of 20 (e.g., about a factor of 10), across the divided portions of the sample. The concentration gradient used for each factor may be the same as or different from the concentration gradients used for other factor(s) in the methods and devices described herein. In some embodiments, the concentration of the coagulation factor added to the divided portions of the sample can be in the range of 0.1 pg/mL to 10 μg/mL. The addition of the coagulation factor at specific concentrations or amounts (e.g., a gradient, such that divided portions have different concentrations) allows the determination of:
a) The presence of an impairment (e.g., a deficiency, which may be due to an abnormality, an inhibition, or a resistance to an inhibitor) of the factor; and
b) Whether any impairment is due to a deficiency in the factor (e.g., because of low factor levels or expression of a dysfunctional factor) or is due to the presence of an inhibitor to the factor (e.g., a drug inhibitor or factor auto-antibodies) or is due to a resistance to an inhibitor (e.g., such as in factor impairments that result in thrombophilia).
   In embodiments, particularly those embodiments differentiating between factor deficiency and factor inhibition, it will be desirable to use, among the factor concentrations or amounts tested, a coagulation factor concentration or amount that falls within the physiologic levels or range for the factor, or to use an amount of the factor that supplies the activity level (often expressed as IU/mL or U/mL) required for normal coagulation, anticoagulation, fibrinolysis, or anti-fibrinolysis, depending on the factor being assessed. In addition, it will be desirable to use at least one factor concentration or amount above the physiologic levels or range, to provide supra-physiologic concentrations or activity levels. Such concentrations and amounts will vary for each factor.

The terms upstream and downstream are used herein to describe coagulation factors that are upstream or downstream in the coagulation pathway (including its associated arms, both fibrinolytic and anticoagulation) with respect to a given point in the pathway (e.g., the point of impairment). The exact pattern of the upstream-downstream combinations may change over time as new data and information are added to the coagulation literature. For example, although Factor XIa is depicted as upstream to FVIII/FVIIIa in the coagulation pathway schema, recently it has been shown that, above certain concentrations, Factor XIa (as well as Factor XI) is capable of bypassing Factor VIII/VIIIa via the direct activation of Factor X (especially in FVIII and FIX deficient plasma, see Kluft et al., Thrombosis Res. 135:198-204 (2015)). This bypassing is discussed in the context of the hemophilia screening assay embodiment in Example 25, where, for a sample deficient in FVIII, adding FXIa at a sufficiently high concentration will shorten the clotting time.

The terms upstream and downstream are also used to describe the activation state of a coagulation factor. For example, Factor IX (the inactive precursor form) would be considered upstream of Factor IXa, and Factor IXa would be considered downstream of Factor IX. Using both the inactive and active forms of a given coagulation factor can increase the specificity of the assay. To illustrate, a user of the devices and methods described herein may determine whether there is a deficiency in Factor IX by using (in the devices and methods) the addition of Factor XIa (which is directly upstream of Factor IX, and is the direct activator of Factor IX). In the case where there is a deficiency of Factor IX, the addition of Factor XIa will not shorten clotting time sufficiently to reach a normal clotting time, and clotting time will remain prolonged. The addition of FIX will shorten the clotting time (in a concentration-dependent fashion, as the exogenously added FIX supplies the factor or replaces any abnormal Factor IX), and the addition of Factor IXa (downstream) will result in an even shorter clotting time. Similarly, certain embodiments of the invention differentiate inhibition of the inactive form from inhibition of the active form of a coagulation factor. Such specific inhibition may be due to drug specificity, such as Factor XI versus Factor XIa inhibitors, or to auto-antibody specificity, such as Factor VIII versus Factor VIIIa auto-antibodies. Differentiating between inhibition of active and inactive forms of a factor may provide clinically relevant information as to the identification of the exact cause of a coagulation impairment and can help guide treatment decisions.

Designating the inactive and active forms of a factor as upstream and downstream relative to each other also applies to the various arms of the coagulation pathway, such as fibrinolysis (e.g., plasminogen is upstream of plasmin and plasmin is downstream of plasminogen) and anticoagulation (e.g., protein C is upstream of activated protein C, and activated protein C is downstream of protein C).

In some embodiments, activators of a specific coagulation factor may be included in addition to the upstream-downstream factors. For example, some embodiments may include a set of lanes that includes plasminogen (upstream of plasmin), plasmin (downstream of plasminogen), and tissue plasminogen activator or urokinase plasminogen activator (an activator of plasmin to plasminogen). In some embodiments, when Factor XIIa is used as an upstream factor, FXIIa can either be added exogenously or Factor XIIa can be generated within the test itself by activation of the contact activation pathway by known activators, such as, e.g., kaolin, celite, glass, certain metals, certain charged surfaces (negatively charged artificial or biological surfaces), and polyanions. This approach will not function if there is an impairment of the endogenous Factor XII in the sample. In such cases, exogenous Factor XII and/or Factor XIIa may be used as part of the upstream-downstream assay. Such cases include subjects with Factor XII deficiency (Hageman Factor Deficiency), which is a common congenital coagulopathy in cats, or in samples containing a Factor XII and/or Factor XIIa inhibitor. In some of the examples listed below, the negative control contains a Factor XII activator.

The positive control lane should include the addition of a downstream coagulation factor. Preferably, the downstream coagulation factor is in the active form, so as to serve as a coagulation activator itself. The use of an activated coagulation factor as the positive control helps eliminate any potential upstream abnormalities or inhibitions affecting the clotting time. For many of the examples described below and in the figures, Factor Xa and/or Factor IIa are frequently used as positive controls. However, the positive control can be any activated downstream coagulation factor. A benefit of using Factor IIa as a positive control includes the ability to verify that the sample is able to form a clot, as the only factor downstream of Factor IIa that would prevent clot formation is Factor I (fibrinogen). Although deficiencies in Factor XIII may result in aberrant or prolonged clotting times, they should not result in the complete absence of a blood clot in the presence of high levels of FIIa and adequate, functional fibrinogen levels. In some cases, a test can have multiple positive controls, such as a test for Factor XI including Factor IXa (downstream and activated) and Factor Xa and/or Factor IIa. If the factor being tested is restricted to activity in the upper portion of the intrinsic coagulation pathway, such as Factor XII, an activator of the extrinsic pathway, such as Factor III, could be used as a positive control, as Factor III would result in activation of the common pathway, downstream of Factor XII.

In some embodiments, a factor that prolongs rather than shortens clotting time may be selected as a positive control, such as in the case of testing for Activated Protein C resistance and using the addition of antithrombin III and/or heparin as a positive control for the appropriate response to an anticoagulant (prolongation of clotting time).

In some embodiments, the presence or absence of clot formation or clot properties may be used in place of, or in addition to the clotting time. For example, the level of tPA may be evaluated via panels that include factors such as plasmin and plasminogen; additionally or alternatively, clot degradation and/or clot integrity may be used as the endpoint. Clot retraction may also be used as an endpoint, for example in the evaluation of Factor XIII function. Another example includes the evaluation of Factor I impairment. In the case of Factor I deficiency (such as afibrinogenemia), or Factor I abnormality (such as dysfibrinogenemia), the clotting time may detect clot or no clot formation as a determination of the presence of a minimum threshold of functional fibrinogen. Quantification of fibrinogen function beyond this minimum threshold may comprise evaluating other clot properties in addition to clotting time.

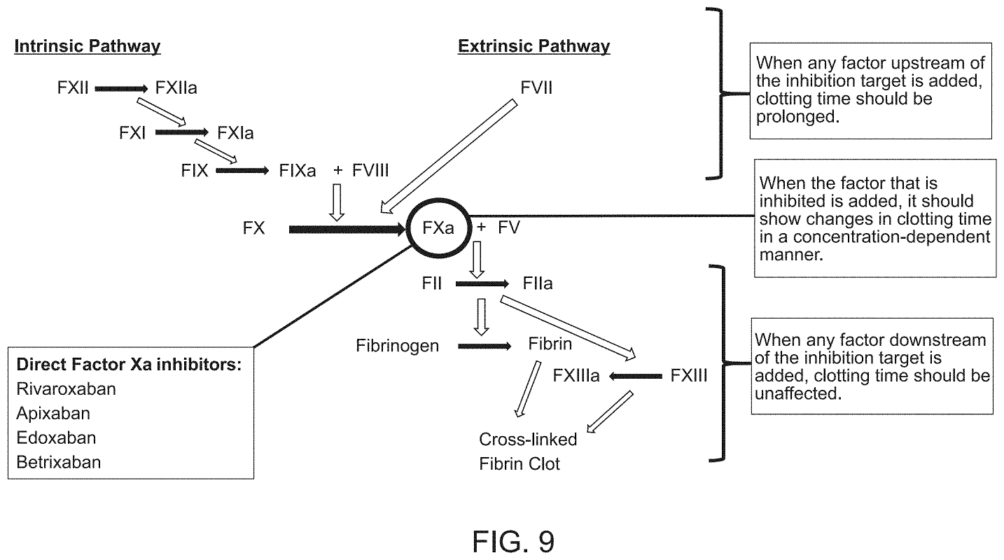

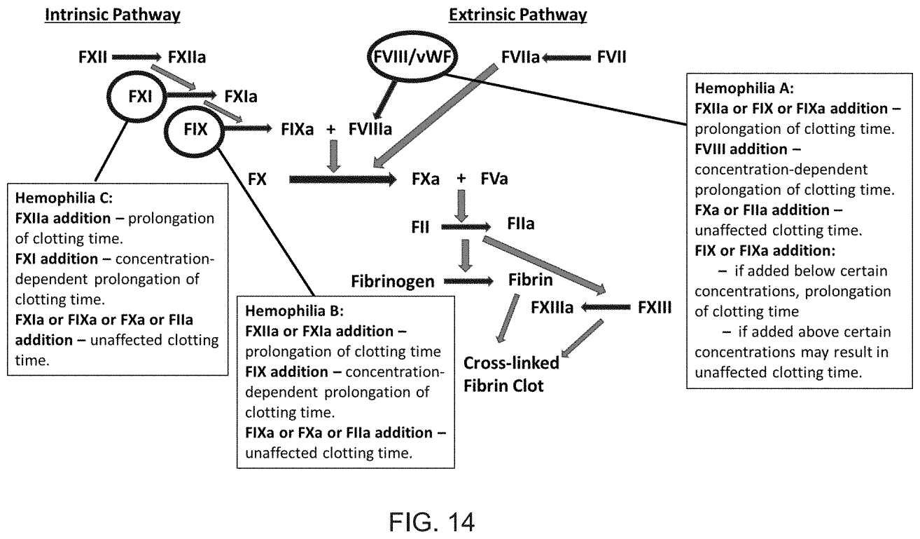

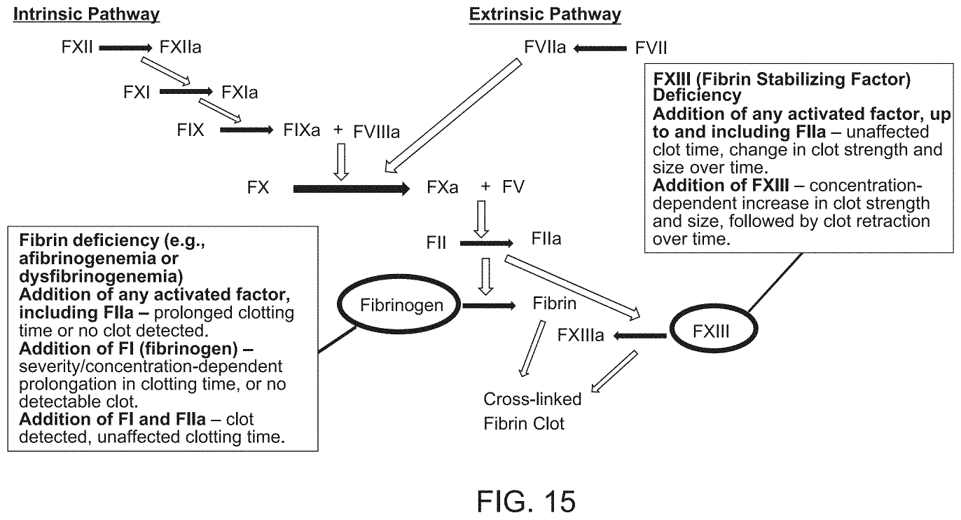

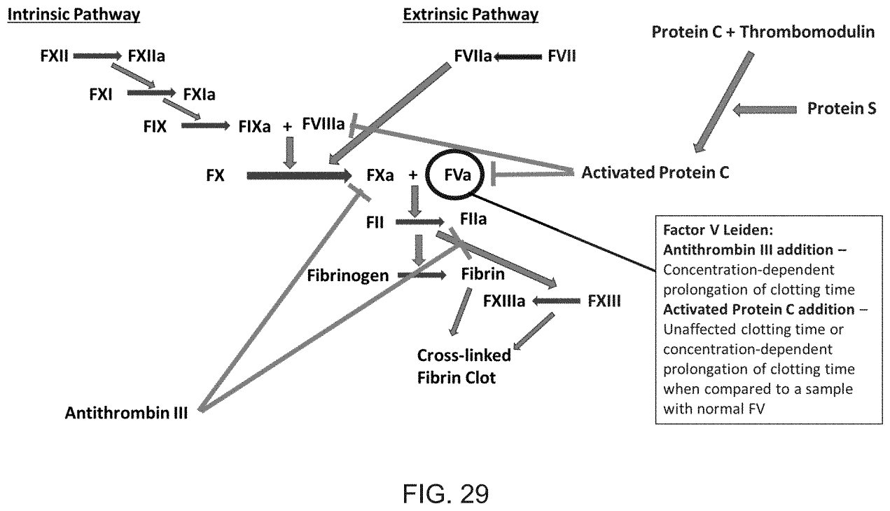

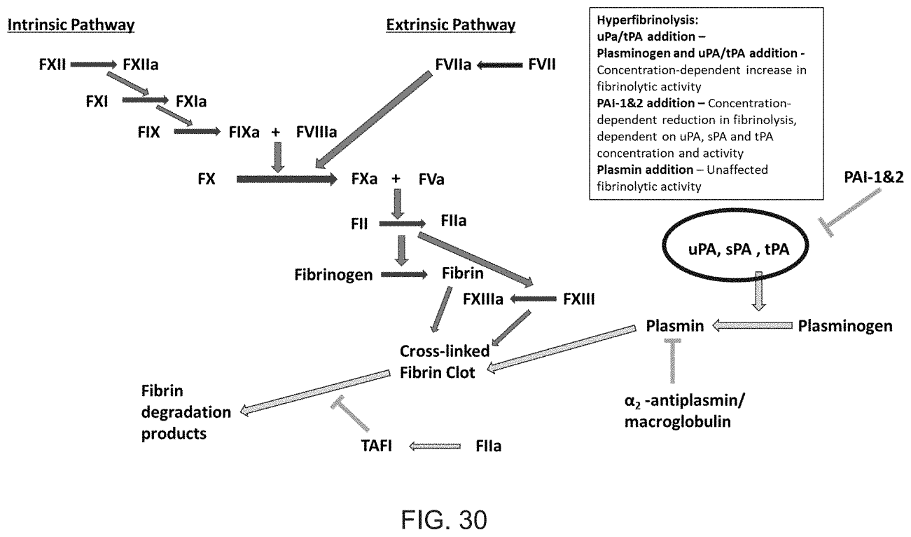

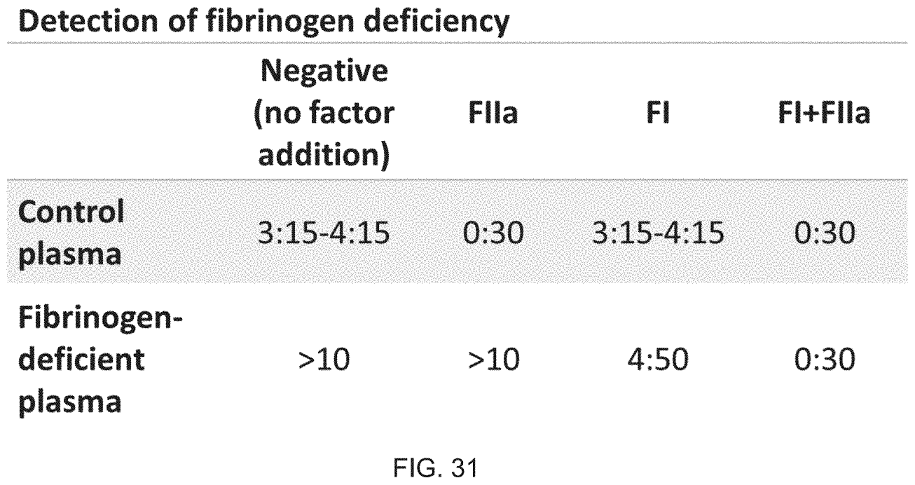

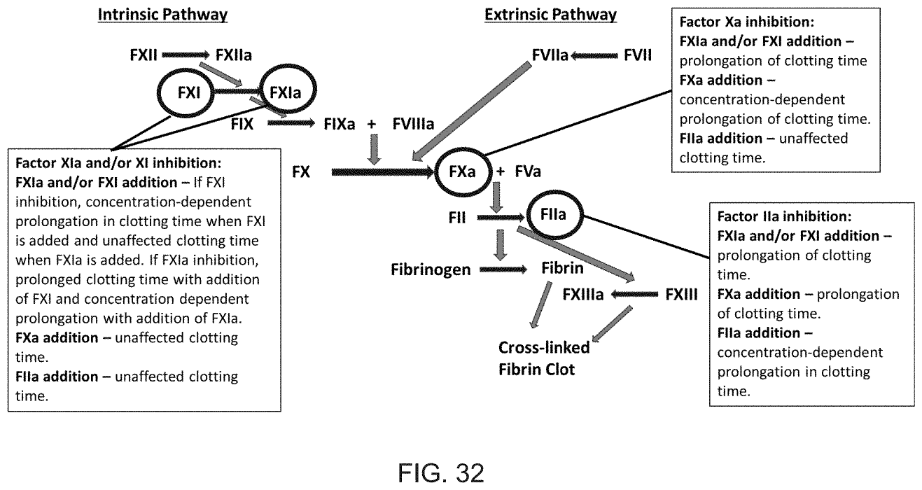

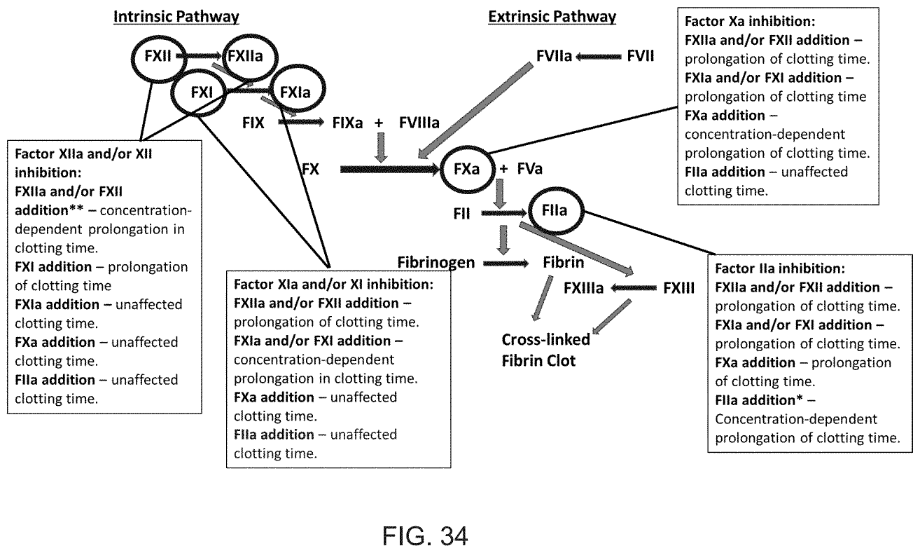

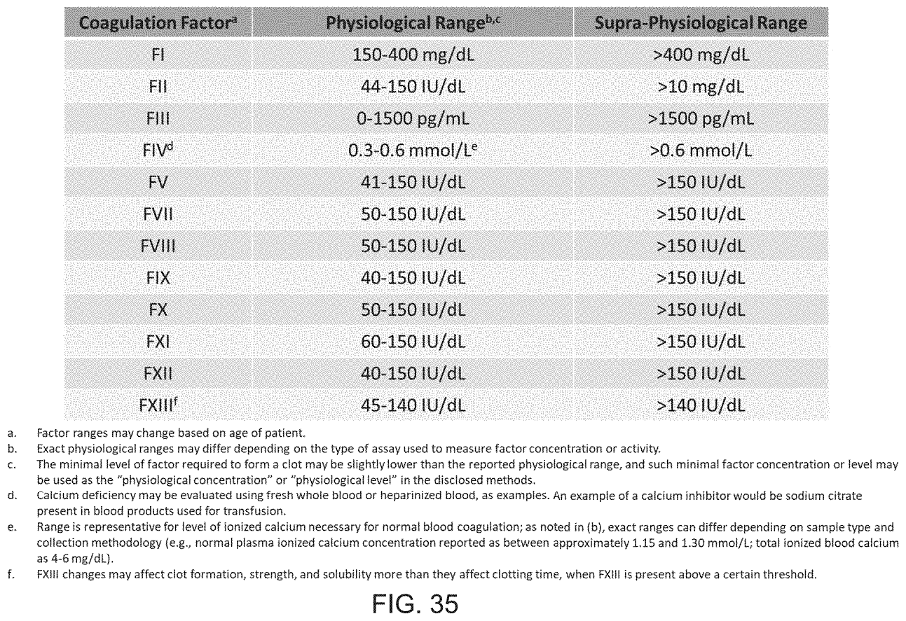

Examples of the utility of the devices and methods described herein include:
a) Detection and/or screening of hemophilia by the addition of various combinations of factors that include Factor XIIa (an upstream factor used as a negative control, as its addition will not decrease clotting time and clotting time remains prolonged), one or more of Factor XI, Factor XIa, Factor IX, Factor IXa, Factor VIII, and Factor VIIIa, and a downstream activated factor as a positive control (such as Factor Xa or Factor IIa); in some embodiments, one or more of the factors are added at various concentrations to provide a factor concentration gradient. See, e.g., FIGS. 14 and 25.
b) Detection of Factor VIII deficiency or inhibition via the addition of Factor VIII as well as upstream and downstream factors, and assessment of Factor VIII inhibition via the addition of Factor VIII at various concentrations (see, e.g., FIGS. 14, 25, 27, and 28). Factor VIII can be supplied as Factor VIII (without other factors) or may be supplied as FVIII in complex with von Willebrand Factor (vWF).
c) Detection of Factor IX deficiency or inhibition via the addition of Factor IX at various concentrations as well as upstream and downstream factors (including FIXa as a downstream factor), and assessment of Factor IXa inhibition via the addition of Factor IXa at various concentrations and upstream and downstream factors (such as FIX as the upstream factor and FXa as the downstream factor and positive control). See, e.g., FIGS. 14, 25, 26, and 27.
d) Detection of Factor XI and/or Factor XIa deficiency or inhibition via the addition of Factor XI as well as upstream and downstream factors and assessment of Factor XI inhibition via the addition of Factor XI at various concentrations. See, e.g., FIGS. 14, 25, 27, 28, 32, 33, and 34.
e) Detection of Factor V Leiden (FVL) disease and detection of Antithrombin III (ATIII) deficiency. FVL is characterized by a resistance to Activated Protein C (APC), a natural anticoagulant. Antithrombin III (ATIII) is an anticoagulant that acts upon FXa and FIIa. Adding various concentrations of ATIII to a sample from a patient with FVL should result in a concentration-dependent prolongation in clotting time (in this case, clotting time should increase as more ATIII is added across portions of the sample). Embodiments of tests for FVL could involve the addition of various concentrations of APC as well as various concentrations of antithrombin III (ATIII) (with individual portions of the sample receiving one of the two factors), and a downstream positive control, such as FIIa, to ensure the ability of the sample to form a clot. If a concentration-dependent prolongation of clotting time is observed with the addition of ATIII but not with APC (or if the concentration-dependent effect of adding APC is less than expected as compared to a reference range, whereas the concentration-dependent effect for ATIII is in line with the reference range), such result would suggest that the specific defect is at the level of APC—in such cases, FVL would be suspected. In some cases, ATIII may be added in conjunction with heparin, which would serve as an anticoagulant propagator and increase the prolongation of clotting time in the test (heparin is an anticoagulant that is also naturally present in the body). In some embodiments, the clotting times can be compared to clotting times of a normal sample or to a reference clotting time, to discern the relative degrees of concentration-dependent effects on clotting times. See, e.g., FIG. 29. ATIII deficiency can be assessed using the same approach. For example, when testing a sample from a patient taking heparin as a therapy for ATIII deficiency, such test may include lanes with Factor IIa and/or Factor Xa. For a normal patient taking heparin these lanes would have prolongations in clotting time reflecting the level of anticoagulation due to heparin, whereas for a patient with ATIII deficiency, such lanes may exhibit shortened clotting times compared to the prolonged clotting times otherwise expected for a patient at that dosage of heparin. ATIII added to some of the Factor IIa and/or Factor Xa lanes should result in a prolongation in clotting time, due to the increased activity of ATIII via the heparin in the sample. In addition, when evaluating for ATIII deficiency, the addition of APC could serve as a control for observing prolonged clotting time. This approach may not be able to differentiate all patients who are ATIII deficient from all other patients who have a marked prothrombotic phenotype and therefore require higher doses of heparin for adequate anticoagulation. In certain embodiments, heparin may be added in addition to ATIII at various concentrations to evaluate the presence of ATIII-resistance.
f) Detection of FVII deficiency or inhibition or FVIIa inhibition. Impairments in FVII can be evaluated by assessing the effects of adding Factor III to activate the extrinsic pathway, which is upstream of FVII; adding Factor III would have either no or minimal effect, such that clotting time remains prolonged. If the deficiency or inhibition is specific to FVII, the addition of FVIIa (in addition to Factor III as the activator) would result in a shortening of clotting time (e.g., clotting time would be unaffected relative to a normal clotting time or other reference clotting time) and the addition of FVII (in addition to Factor III as the activator) would result in a concentration-dependent effect on clotting time. In the presence of a FVIIa inhibitor (depending on level of inhibition) the addition of FVIIa would result in a concentration-dependent shortening of the clotting time.
g) Detection of FXII deficiency or inhibition. Embodiments of the present invention designed for detecting FXII impairments may include a negative control lane that contains a portion of the test sample and a contact activator to activate Factor XII to Factor XIIa. Clot formation from this negative control can be compared to clot formation in a lane containing test sample and exogenous FXIIa. In the case of FXII deficiency, the addition of FXIIa would result an unaffected clotting time (clotting time would be shortened to, e.g., the clotting time range of a normal sample or of another reference) while there would be prolonged clotting time in the negative control lane, and the addition of FXII would result in a concentration-dependent shortening of the clotting time. In the case of a FXII inhibitor, the results would be similar to the above, with higher concentrations of FXII being required to shorten clotting time for those lanes where FXII is added. In the case of a FXIIa inhibitor, the addition of FXIIa would result in a concentration-dependent effect on clotting time, rather than in unaffected clotting time. A downstream positive control (e.g., activated coagulation factor such as FXIa, FIXa, FVIIIa, FXa or FIIa) may also be included. See, e.g., FIG. 34.
h) Detection of Factor I deficiency. Addition of FIIa at high concentrations to a sample should result in the generation of a blood clot. However, if no blood clot is formed or if blood clotting time is prolonged (compared to, e.g., a normal sample's clotting time or another reference clotting time), the lack of a clot or prolonged clotting time is likely due to a deficiency (e.g., an abnormality resulting in reduced function) of fibrinogen in the sample. For example, in certain embodiments, addition of Factor IIa will result in weak or no clot formation (e.g., within a 10-minute test window) when fibrinogen is below 150 mg/dL or lacks the functional equivalent of this concentration. The addition of fibrinogen to the sample, so that the sample has sufficient fibrinogen for clot formation, would result in the formation of the blood clot (where no blood clot was observed without adding fibrinogen) or in a shortening of clotting time, depending on the severity of the deficiency and the concentration of the added fibrinogen. Further, if a blood clot does not form with FIIa addition but does form when both FIIa and FI are added to the sample, such result indicates a deficiency (e.g., abnormality) in FI. Adding both FIIa and FI to a single portion of the sample (the positive control in this test sequence) allows for the specific identification of a FI deficiency, and for differentiating such deficiency from a FIIa impairment (such as FIIa inhibition). See, e.g., FIGS. 15 and 31. Further, in embodiments where data characterizing the blood clot, such as data regarding blood clot strength, are collected, then the addition of functional FI should also result in an increase in clot strength. Unlike the traditional Thrombin Time (TT) testing approach, where prolongation of TT suggests a problem with fibrinogen or thrombin inhibition, the multi-factor testing approach described herein permits the identification of a fibrinogen deficiency and the differentiation between thrombin inhibition and a fibrinogen deficiency (e.g., abnormality).
i) Detection of Factor IIa, Factor Xa, Factor XI, and/or Factor XIa inhibition. In some embodiments, various concentrations of Factor IIa and Factor Xa are utilized for the detection of Factor IIa inhibition and/or the detection of Factor Xa inhibition. Additional tests can be used with this set of assays, including tests to determine the effect(s) of adding various concentrations of Factor XI and/or of adding various concentrations of Factor XIa. Using multiple factors, including factors in activated form, and using various concentrations of the factors, permits the detection and quantification of Factor XI and/or of Factor XIa inhibition. Additionally, such methods, and devices configured for such methods, can be used to differentiate among Factor Xa inhibitors, Factor IIa inhibitors, Factor XI inhibitors, and/or Factor XIa inhibitors. See, e.g., FIGS. 32-34.
j) Detection of Factor IIa, Factor Xa, Factor XII, and/or Factor XIIa inhibition. In some embodiments, various concentrations of Factor IIa and Factor Xa are utilized for the detection of Factor IIa inhibition, and/or the detection of Factor Xa inhibition. Additional tests can be included with this set of assays, including tests to determine the effect(s) of adding various concentrations of Factor XII and/or of adding various concentrations of Factor XIIa. Using multiple factors, including factors in activated form, and using various concentrations of the factors, permits the detection and quantification of Factor XII inhibition and/or of Factor XIIa inhibition. Additionally, such methods, and devices configured for such methods, can be used to differentiate among Factor Xa inhibitors, Factor IIa inhibitors, Factor XII inhibitors, and/or Factor XIIa inhibitors. See, e.g., FIGS. 32-34.
k) Detection of Factor IIa, Factor Xa, Factor XI, Factor XIa, Factor XII, and Factor XIIa inhibition. In these embodiments, various concentrations of Factor IIa and Factor Xa are utilized for the detection of Factor IIa inhibitors and of Factor Xa inhibitors. Additional tests can be included with this set of assays, including tests involving the addition of various concentrations of Factor XI and/or Factor XIa, and the addition of various concentrations of Factor XII and/or Factor XIIa. Such an approach permits the detection and quantification of Factor XI inhibition and of Factor XIa inhibition, and of Factor XII inhibition and of Factor XIIa inhibition. Additionally, such methods, and devices configured for such methods, can be used to differentiate among inhibitors specific to these factors.
l) The detection of hypofibrinolysis, secondary to deficient or inhibited plasminogen or excess plasminogen activator inhibitor (PAI), and detection of hyperfibrinolysis, secondary to active urokinase/streptokinase (uPA/sPA), or tissue plasminogen activator (tPA) (either naturally occurring or due to fibrinolytic drug administration). uPA/sPA and tPA each activate plasminogen to plasmin, resulting in degradation of the cross-linked fibrin clot into fibrin degradation products (FDPs). As discussed above in various embodiments, the addition of FIIa or another activator (such as FXa) is used to stimulate the formation of a cross-linked fibrin clot and serve as a positive control of clot formation. In an analogous fashion, the addition of plasmin can serve as the positive control for fibrinolysis, where the addition of exogenous plasmin results in an increased breakdown of the clot in a concentration-dependent manner. The addition of excess plasminogen provides a substrate for any endogenous or exogenous uPA/sPA or tPA and serves as an additional control in cases where there is a plasminogen deficiency or inhibition. The addition of naturally-occurring fibrinolysis inhibitors should result in a concentration-dependent decrease in fibrinolysis, which, when compared to a reference/control range, can potentially identify increased levels and/or activity of fibrinolysis activators (tPA, uPA, etc.). This approach can be used to detect naturally-occurring hyperfibrinolytic states, such as trauma-induced coagulopathy, or can be used to detect and quantify the fibrinolytic activity of fibrinolytic drugs, such as tPA administered during stroke treatment. See, e.g., FIG. 30. This same approach can be used to explore other fibrinolytic substances or anti-fibrinolytic substances, such as plasminogen activator inhibitor 1&2 (PAI-1&2), thrombin-activatable fibrinolysis inhibitor (TAFI), and many others in order to also assess hypofibrinolysis. In the evaluation of fibrinolysis, other blood clotting parameters, such as clot strength and clot retraction, may be assessed, in addition to clot time. Detection of a decrease in fibrinogen quantity or function can also be detected using the upstream-downstream methods described herein.
m) Detection of other coagulation factor impairments via the addition of a coagulation factor that is impaired (e.g., the factor is present at sub-physiologic levels in the sample, has an abnormality impairing function, and/or is inhibited), and its upstream and downstream factors. Upstream and downstream factors may include a different coagulation factor and/or the activated or inactivated form of the coagulation factor. See, e.g., FIGS. 9-15, 25-34.

Embodiments of the methods and devices described herein can be used to evaluate coagulation impairments (e.g., pro- or anti-thrombotic) using various coagulation detection technologies that can evaluate the multiple parts of the coagulation system, including coagulation cascade, anticoagulation, platelet activation, and fibrinolysis, such as those described herein. Detection methods include, for example, detection based on light absorbance, fluorescence measurements, ultrasound, etc., and the detection device can be configured to employ one or more of these other methodologies.

Whole blood or plasma can be used in the various embodiments described herein. In addition, the devices and methods described herein can be applied to other bodily fluids, including, for example, cerebral spinal fluid, amniotic fluid, peritoneal fluid, pericardial fluid, pleural fluid, and any other fluid sample that may contain coagulation factors.

Embodiments provided herein can be combined with ATP-luciferase assays in order to assess platelet and coagulation system function at the same time. Using the methods and devices described herein with ATP-luciferase assays can provide evaluation of the coagulation cascade, as well as platelet function, via the degranulation of the platelet upon sufficient activation. Activation of the platelet can occur via the addition of the coagulation factors discussed herein, or by the addition of specific platelet agonists, such as, e.g., adenosine diphosphate (ADP), adenosine triphosphate (ATP), epinephrine, collagen, thrombin, and ristocetin. These agonists can be added as a concentration gradient in combination with the coagulation factors. This combined technique can be used to assess platelet function when patients are taking platelet inhibitors, such as aspirin or clopidogrel. Luciferase is typically measured by light absorbance. Platelet function can be measured via other detection methodologies, such as assessing platelet aggregometry by electrical impedance or light absorbance.

Coagulation impairments that can be detected or analyzed include, but are not limited to, congenital or hereditary coagulopathies and acquired coagulopathies.

Congenital or hereditary coagulopathies include acquired mutations and hereditary coagulopathies, i.e., inherited from a parent.

Congenital coagulopathies are present at birth and are likely due to a developmental abnormality that occurred in utero. Congenital coagulopathies may or may not be genetic. In some embodiments, a patient may have or be suspected to have a coagulation factor deficiency, which may be caused, for example, by the production of a deficient amount of the coagulation factor, or a mutation in the coagulation factor that decreases the factor's function of the factor, or both.

Examples of congenital coagulopathies include, but are not limited to:
a) Hemophilia A (Factor VIII deficiency)
b) Hemophilia B (Factor IX deficiency)
c) Hemophilia C (Factor XI deficiency)
d) Factor XII deficiency
e) Factor I (fibrinogen) deficiency
f) Factor V deficiency
g) Factor V Leiden (FVL) thrombophilia
h) Factor VII deficiency
i) Factor X deficiency
j) Factor XIII deficiency
k) Antithrombin III deficiency
l) Alpha2-antitrypsin deficiency
m) Alpha1-antitrypsin deficiency
n) Combined factor deficiencies (e.g., Factor V and VIII, Factor II, VII, IX, and X)
o) Platelet abnormalities (e.g., Gray platelet syndrome, Bernard-Soulier syndrome, von Willebrand disease, Glanzmann thrombasthenia, Hermansky-Pudlak syndrome, clopidogrel or aspirin resistance).

Causes of acquired coagulopathies include, but are not limited to: organ (e.g., liver) dysfunction or failure, bone marrow dysfunction or failure, trauma (e.g., automobile accident), surgery, infection (e.g., coronavirus, flavivirus, hemolytic uremic syndrome, sepsis, etc.), cancer, immobility, drugs (e.g., antibiotics, anticoagulation, fibrinolytics, thrombolytics, chemotherapy, fluids, etc.), neutraceuticals/pharmaceuticals, toxicities, envenomation (e.g., snake, spider, etc.), foods, auto-immune diseases (whether primary, acquired or idiopathic), implants (e.g., surgical), cardiovascular event(s) (e.g., a clot of blood anywhere in the body, including stroke, heart attack, etc.), vasculitis, transfusions (e.g., whole blood, packed red blood cells, plasma, platelets, etc.), transplants (e.g., bone marrow, kidney, liver, etc.), pregnancy (e.g., pre-eclampsia, eclampsia, diabetes, etc.), endocrine disease (e.g., pheochromocytoma, cushing syndrome, diabetes, etc.), chronic inflammatory disease (e.g., irritable bowel syndrome, irritable, bowel disease, colitis, etc.), and disseminated intravascular coagulation, and radiation.

Coagulopathies may also be iatrogenic (e.g., caused by medical treatment, such as some cancer treatments, bone marrow transplant, and certain drug treatments) or may be due to idiopathic causes.

In some embodiments, the invention employs a microfluidic approach. In certain embodiments, the microfluidic device includes a series of channels in a substrate, each channel having an area with a geometry to trigger and/or localize formation of a clot, to allow for evaluation of clot formation in response to one or more reagents, such as the amount or concentration of an exogenously added coagulation factor. Each of the channels in the series has the same geometry, so as to trigger identical clot formation properties (when exposed to the same sample and reagents). By evaluating clot formation in the presence of a gradient of one or more coagulation factors, the invention allows for sensitive and specific detection of coagulation abnormalities or impairments, as described above.

Embodiments employing a microfluidic device, may involve the following procedures:
a) A sample is acquired from a patient;
b) One or more agonists (specific factor(s)) is/are added to the patient sample as described herein (either before entry into the microfluidic device or within the microfluidic device), each agonist at an increasing concentration across a series of channels in the microfluidic device; for example, the various embodiments described herein include methods wherein the factors are added to the sample within the microfluidic device after sample entry, either by being contained within the microfluidic device before sample entry and then being added to the sample after and as a result of sample entry, or by being inputted into the device after sample entry;
c) +/− calcium is added if the sample is collected in an anticoagulant, such as sodium citrate or acid citrate dextrose;
d) The sample then flows through the microfluidic device where formation of a clot is triggered at a specific location within the channels;
e) The time to clot is measured and/or quantified at the location, and then recorded;
f) Multiple concentrations of the same agonist may be added to the aliquoted sample (in separate channels) to determine the presence and concentration of a coagulation cascade impairment; concentrations can (but need not necessarily) range, for example, from about 0.1 pg/mL to about 10 μg/mL, or, for example, from about 0.10 μg/mL to about 1000 μg/mL;
g) Multiple factors may be added to the aliquoted sample (in separate channels) to identify the part of the coagulation cascade that is impaired. By utilizing upstream and downstream factors, such as the use of Factor IIa and Factor Xa in the identification of DOACs, or the use of Factor VIII, Factor IX, Factor IXa, Factor XI, and Factor XIa in the identification of Hemophilia A, B, or C, a user of the methods and devices provided herein can identify the point at which normal clotting is recovered. Another example embodiment is identification of a concurrent dysfibrinogenemia or afibrinogenemia: with a whole blood or plasma sample, the negative control lane (no agonist added) may exhibit prolonged clotting times, while addition of downstream coagulation factors (such as Factors IIa and Xa) will not recover normal clotting times, the addition of fibrinogen (Factor I) to the sample recovers normal clotting times since this missing/abnormal factor is being replaced in the device.

A microfluidic device for detecting coagulation can include plural channels formed in a substrate, each channel including a clot forming area having a geometry configured to trigger and/or localize formation of a clot. In some embodiments, the clot forming areas of the plural channels are arranged in a central region of the substrate. In some embodiments, the device further includes plural sample input ports, each sample input port connected to a first end of one of the plural channels. In some embodiments, the device comprises plural output ports, each output port connected to a second end of one of the plural channels. The input and output ports may be arranged in an alternating pattern at a periphery of the substrate. In some embodiments, the device comprises a common sample input port, in fluid connection with all channels or a series of channels.

A substrate can be, for example, any type of plastic, polydimethylsiloxane (PDMS), silicon, glass, polyimide, or other material or combination of materials. In an embodiment, the device includes a substrate bound to glass, but other substrates can be used, such as glass on glass, PDMS on PDMS, silicon, any type of plastic, or combinations thereof. In one embodiment, the substrate is plastic. The substrate can be (but need not be) transparent to facilitate the detection of clot formation (vis-à-vis, e.g., imaging).

The device can include microfluidic channels with a diameter of about 50 μm, a height of about 11 μm, and a length of 100+μm. Other channel dimensions can be employed.

One entry and one exit port for the sample input can be provided for each channel. Alternatively, devices can provide a single sample port for all channels or for one or more groups (or series) of channels.

In various embodiments, an agonist (e.g., a coagulation factor) is added to the sample prior to input into the device or the agonist is coated to, or otherwise pre-loaded within, the device prior to sample loading. In the case where one or more channels include the coagulation factor(s), the coagulation factor(s) may be in suspension, solution, or lyophilized, and may be surface-bound or not surface-bound. The coagulation factor(s) can be pre-included in the channel(s) (e.g., at the time of manufacturing the device), can be added prior to placing the sample into the device, or can be entered into the device through an input port (or multiple input ports) simultaneously with the sample or after the sample.

In some embodiments, calcium is added to the sample. Calcium may be added to the sample prior to inputting the sample into the device. In addition, calcium can be added within the device, e.g., through an additional port, or pre-loaded within the channel.

In some embodiments, the sample is loaded into the device or microfluidic cartridge via capillary action. The sample can also be forced to flow through the channel, e.g., through the use of a vacuum, syringe-pump, or other suitable means, including, in some embodiments, gravity. The sample can also be encouraged to load by capillary action or flow by using coating that alters the surface properties of the microfluidic device (e.g., substrate), such as by making it hydrophilic.

In certain embodiments, the design of the microfluidic channel(s) includes one area of an altered geometry (including different angled bends and/or diameters) in order to create one area of flow separation and stasis to trigger and/or localize formation of the clot. The time that it takes for the clot to form can be quantified and recorded (e.g., reported to the user).

In various embodiments as described herein, the device is used to detect the presence and assess the effect of coagulation factor deficiencies or inhibition by assessing the time is takes to form a clot.

In some embodiments, a microfluidics device is used to detect the presence of and assess the effect of coagulation factor deficiencies or inhibition by assessing the time is takes to form a clot and/or by evaluation of other blood clot formation parameters, such as clot strength, clot retraction, or clot degradation.

In certain embodiments, a device as described herein provides a read-out (e.g., of clot formation time(s)) in a relatively short period of time, for example, in about 3-10 minutes.

Example microfluidic devices and assays are described below and illustrated in the figures.

### EXAMPLES

### Example 1

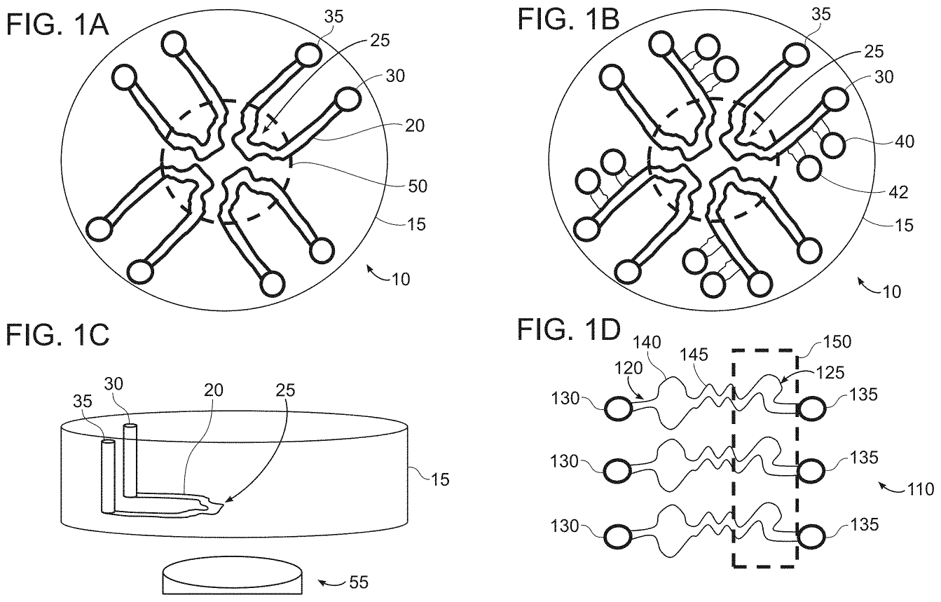

FIGS. 1A-1D are schematic illustrations of microfluidic device layouts according to example embodiments of the invention.

FIG. 1A is a top view of a circular layout (it can also be any symmetrical polygon with a center point) of microfluidic device 10 having one or more continuous microfluidic channels (e.g., microchannels) 20 formed in a substrate 15, each channel connected to one inlet (input port) 30 and one outlet (output port) 35. A portion of the channel, e.g., the center of the channel, can have a unique shape, e.g., a clot forming/localizing area 25, in order to result in flow separation or disruption, or stasis of sample flow to promote clot formation. There may be two or more of these microfluidic channels in this single device, dependent on the specific assay being used. This design can allow for multiple samples, such as three or more samples, e.g., up to 10 samples, or more than 10 samples, to be evaluated simultaneously. Typically, each sample (or each aliquot of a sample) requires a separate channel. In FIG. 1A, four channels are illustrated, each having a clot forming/localizing area 25 located proximally on the microfluidic device, e.g., locate in a central region of the microfluidic device. The sample can enter the device through the inlet manually or by an electronic dispenser and will go through the microfluidic channel by an applied pressure/vacuum, capillary action, or via chemical interactions, such as if the microfluidic channel is coated with or made of a hydrophilic material. In this example set-up, the agonists +/− calcium +/− clot detection reagents must be added to the main inlet, pre-mixed into the sample, or must be coated on to the inlet or the microfluidic channel. (The term “+/−”, as used herein means “with or without.”) All of the clot forming/localizing areas may be viewed in one single imaging field (dashed circle 50 encompassing clot forming areas 25) at magnification that may range from, for example, 2×-10×.

FIG. 1B is a top view of a similar layout as in FIG. 1A but with examples of multiple inlet ports 30, 40, 42 for each channel 20. This allows for the agonist +/− calcium +/− clot detection reagents to be added to the sample within the microfluidic channel. There can be one or more additional inputs 40, 42 and they can individually connect directly to the main channel 20 or the main input area, or some may connect indirectly to each other with at least one connecting to the main channel or the primary input port.

FIG. 1C is a side view of a microfluidic device layout illustrating input 30 and output 35 ports of a channel 20 in substrate 15. Only one channel is shown, but one or more channels may be provided as illustrated in FIG. 1A. In addition, one or more input ports may be provided for each channel, as illustrated in FIG. 1B. As schematically illustrated in FIG. 1C, a detection device 55 can be provided to measure clot formation in each of the channels. The detection device 55 can include an imaging sensor to detect clot formation, e.g., clot formation times. Imaging can be bright-field imaging as described herein. The detection device may use any of the other measuring/detection methodologies described herein.

FIG. 1D is a top view of a microfluidic device 110 having an alternate layout that may be utilized for various assays. There can be one or more inlets (input ports) 130 with one outlet (output port) 135 per sample input and channel 120. An area of shape change 125 to stimulate clot formation is included in each channel 120. The channels are arranged in a parallel fashion in order to allow for visualization of the clot formation/localization areas 125 within one field of view (dashed rectangle 150) at magnification that may range, for example, from 2×-10×. Each channel can include one or more areas 140 for agonist and/or calcium addition and a region 145 for mixing. In the example shown, the channels 120 have identical geometries.

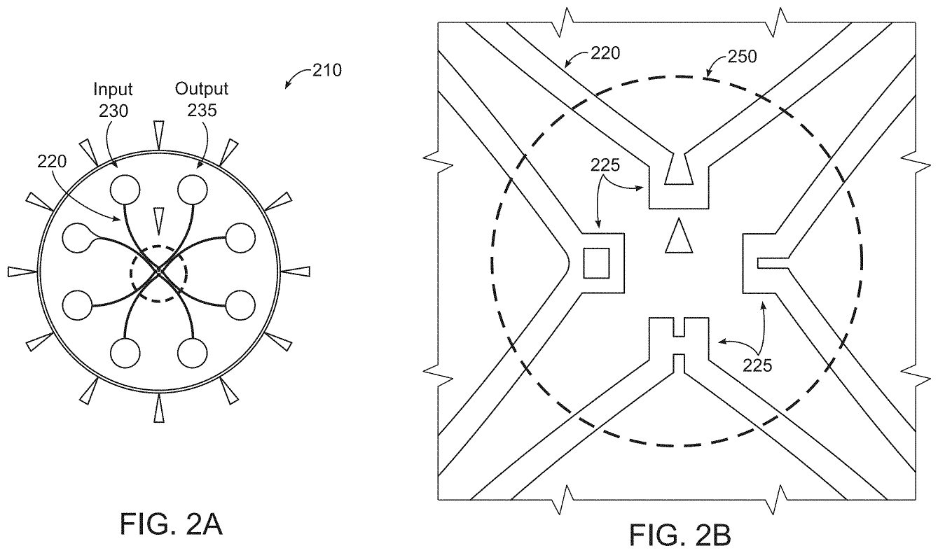

FIGS. 2A and 2B illustrate a circular microfluidic clotting device 210 according to an example embodiment. As shown, the device includes four channels 220, each channel including a clot forming/localizing area 225 having a geometry to trigger and/or localize clot formation. The clot forming areas 225 are arranged in a central region. Each channel 220 is connected to an input port 230 and an output port 235. The input and output ports of all the channels are arranged in an alternating pattern at a periphery of the device 210. The dashed circle 250 in the center indicates a general field of view encompassing ‘clotting areas’ 225 of all input channels. The configuration of channels shown in FIG. 2A is a configuration in which wicking capillary flow occurs, but many other configurations are possible. A particular configuration may be selected based on one or more criteria, such as whether the configuration is particular advantageous for manufacturing the device.

FIG. 2B is a magnified view of the central portion of the device 210 of FIG. 2A illustrating examples of clot forming/localizing areas 225 within the field of view. The clot forming areas can have configurations conducive to formation of a clot that can be quantified. The clot forming areas can have shapes designed to cause flow separation, stasis, flow disturbances, or combinations thereof, for clot formation, and may have shapes designed to cause flow disturbance for clot formation. In the example, the clot forming areas have different shapes to illustrate various shapes that can be used. Typically, the shapes will be the same for each channel so as to ensure the same flow conditions in each channel. The shapes of clot forming areas illustrated in FIG. 2B are examples and not all-inclusive of the shape variations that can be used.

As illustrated in FIG. 2B, each clot forming area can be configured (e.g., shaped) such that a sample flowing through a clot forming area is forced to change direction at least once, preferably multiple times. Each change in direction can be in the range of, for example, about 45 degrees to about 135 degrees, of about 60 degrees to about 120 degrees, of about 75 degrees to about 105 degrees, or of about 90 degrees. In addition, one or more flow disruptors, such as protrusions or islands, can be provided to disrupt flow. As a sample passes through the clot forming area, it encounters flow disruptor(s) and is forced to flow around the disruptor(s). A disruptor may include corners or pointed edges, and can be triangular, rectangular, or otherwise shaped as illustrated in FIG. 2B. A combination of disruptors and other structural features, or just other structural features, may form a circulatory region, where sample flow in a circular pattern interacts with new sample entering the region as other sample departs. Eddy currents behind disruptors, from a fluid flow point of view, may also encourage coagulation as sample interacts with other sample at intersections (e.g., turbulence intersections) of fluid flow and sample in an eddy region.

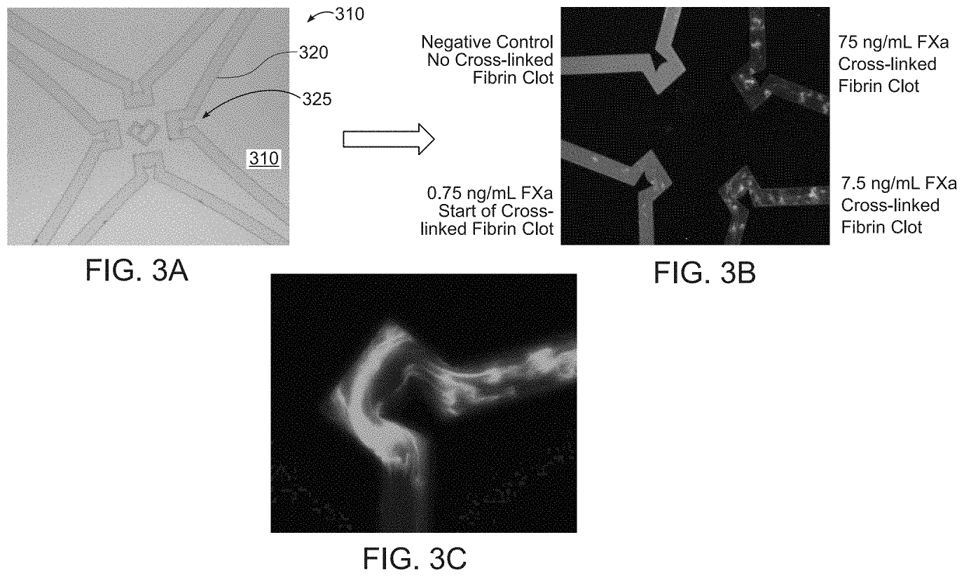

In some embodiments, the disruptor can include a concavity (e.g., FIG. 3A). A clot forming/localizing area may include a narrowing of the channel. By changing the direction of sample flow and/or changes in diameter, angle, and/or shape of the channel, and/or forcing the sample to flow around one or more disruptors, the clot forming areas introduce flow separation and stasis of sample flow to promote clot formation. Typically, the channels and clot forming areas are arranged in a symmetrical pattern in order to provide the same flow characteristic for each of the channels.

### Example 2

A general protocol for performing the assay according to an embodiment of the invention is as follows:
a) Add together sample, agonist, +/− calcium, +/− clot detection agent
   i. Calcium to a final concentration between 0.005-0.05 M (This concentration is particularly suitable for use with 3.2% buffered sodium citrate. If another anticoagulant is used, the concentration of calcium may not be 0.02 M.)
   ii. Clot detection agents can include fluorescent labeled fibrinogen, magnets, beads (may be fluorescent or colored)
b) Load into microfluidic device
   i. See, e.g., FIGS. 1A-1D, 2A and 2B for examples of input loading configuration and order
c) Temperature control
   i. Room temperature
   ii. May increase up to 37° C. (body temperature) (Body temperature is typically 37° C. but the temperature of the assay run can be changed according to the patient's actual temperature. For example, if a patient has a fever, the temperature of the assay run can be increased or if the patient is hypothermic the temperature of the assay can be decreased.)
d) Perform clot detection and measure time of clot formation (e.g., 4-12 minutes)
e) Log time when each sample starts to form a clot

### Example 3

FIGS. 3A-3C illustrates clot detection using plasma and fluorescent-labeled fibrinogen with a microfluidic device 310 having four channels 320 with clot forming/localizing areas 225 according to an example embodiment. The microfluidic device is similar to the device show in FIGS. 2A and 2B except that all clot forming areas 325 have the same shape. Each clot forming/localizing area 325 includes a protrusion to disrupt sample flow. In this example, as shown in FIG. 3A, the protrusion generally is triangular in shape. Two sides of the protrusion are straight and one side is concave. Each clot forming area 325 causes the flow to change direction four times, including two 90 degree changes in direction.

In an example, the process of clot detection can include the following procedural steps:
a) A plasma sample is pre-mixed to include: 6 μL plasma+0.6 μL agonist (10% volume to sample)+0.6 μL Calcium (stock 200 mM, 10% volume to sample)+0.6 μL Fibrinogen (this can vary in concentration, in general <10% volume of sample). The foregoing values can be adjusted and changed and similar results obtained.
b) For each channel, an aliquot of the pre-mixed sample is placed into the input port of the channel.
c) The sample aliquot is drawn into the channel by capillary action.
d) The channels are imaged for 10 minutes at 37° C., and the time to detect a clot is recorded.

The example in FIG. 3B shows a fluorescent image taken of the microfluidic channels at one time point (5 minutes). The plasma sample used contains 250 ng/mL of Apixaban. An agonist, Factor Xa (FXa) at various concentrations (0.75 ng/mL FXa, 7.5 ng/mL FXa, and 75 ng/mL FXa; units “ng/mL” refer to ng×10³/mL) or buffer alone (negative control) was added to the plasma sample, along with calcium and 488-conjugated fibrinogen. Crosslinking of the fluorescent fibrinogen is indicative of the formation and presence of a cross-linked fibrin clot. Higher concentrations of the FXa (7.5 ng/mL FXa, and 75 ng/mL FXa; units “ng/mL” refer to ng×10³/mL), visible in the channels on the right in FIG. 3B, result in clot formation earlier than the lower concentration (0.75 ng/mL FXa; units “ng/mL” refer to ng×10³/mL) or the negative control, visible in the channels on the left in FIG. 3B. FIG. 3C is a magnified view of a clot forming area of one channel illustrating a cross-linked fibrin clot.

### Example 4

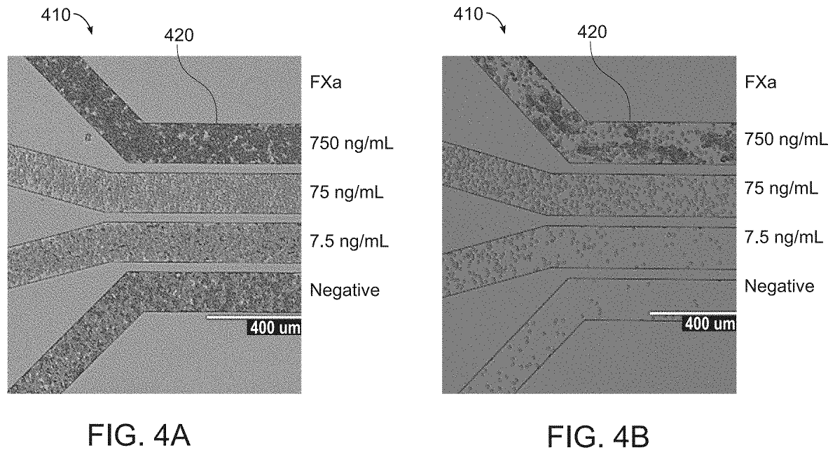

FIGS. 4A and 4B are fluorescent images illustrating clot detection using whole blood in a parallel microfluidic channel device 410 according to an example embodiment. Microfluidic channels 420 were pre-coated with agonist, Factor Xa, at various concentrations (7.5 ng/mL, 75 ng/mL, 750 ng/mL; units “ng/mL” refer to ng×10³/mL) or with buffer alone (negative control). The fluorescent images are taken at one time point (10 minutes). Microfluidic channels were washed with buffer prior to use to leave only bound FXa within the microfluidic channel. Fresh whole blood was placed into each input port and the blood was drawn in through capillary action. The blood was left to flow for 10 minutes and then the channel was gently washed with buffer. Depicted is a brightfield image of two samples evaluated. The sample in FIG. 4A contained no anticoagulant (finger prick of blood), which resulted in clots in all 4 channels, including the negative control. The sample in FIG. 4B contained unfractionated heparin (which was added to the finger prick of blood), which resulted in a gradient of clot formation dependent on the concentration of FXa in the channel. Almost no cells were adhered in the negative control, indicating minimal clot formation. Unfractionated heparin inhibits Factors IIa and Xa in an antithrombin III-dependent fashion, which is why the addition of these factors at appropriate concentrations can help recover the clotting capability of the sample.

### Example 5

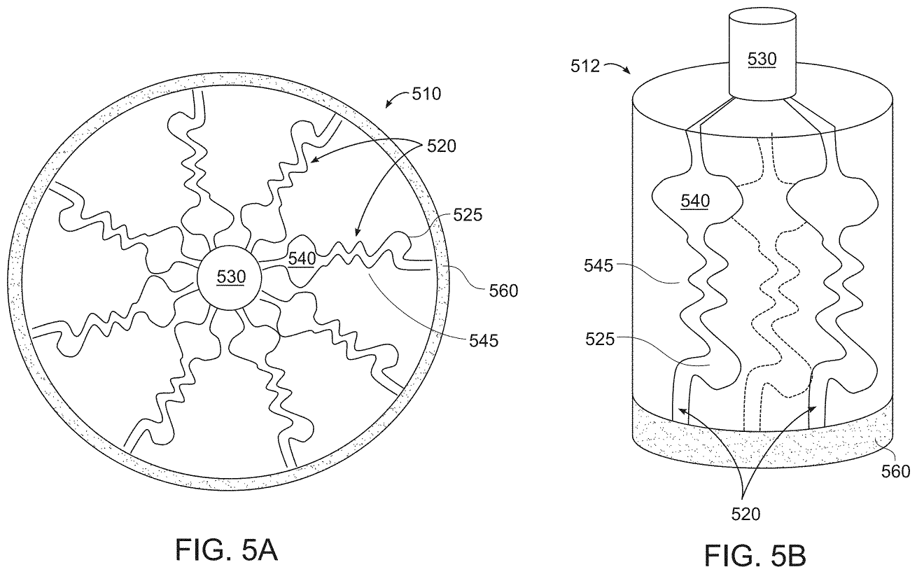

FIGS. 5A and 5B illustrate additional embodiments of microfluidic device designs that include the features of: (1) each channel subjects the blood/plasma to equal conditions and (2) there is a clot-promoting geometry within each channel where clotting detection is optimized and performed. FIG. 5A illustrates a device 510 including circular array of symmetrical channels 520 surrounding and connected to a single sample input 530, where each channel has a clot-promoting and/or localizing area 525. The channels 520 may or may not also include one or more areas for agonist and/or calcium addition 540 and/or mixing 545. FIG. 5B illustrates an alternative embodiment of a device 512 utilizing a cylindrical design with a single sample input port 530 that divides into multiple symmetrical channels 520 with a clot-forming area 525 with or without an area for agonist/calcium 540 addition and/or mixing 545. Both devices 510, 512 may also include a sample collection reservoir 560 with or without an absorbent filter.

### Example 6

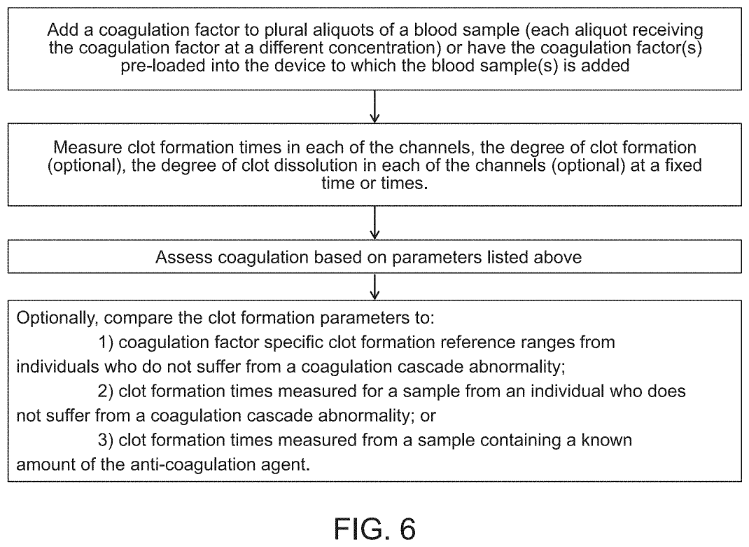

FIG. 6 is a flow diagram of a method of assessing coagulation in a blood sample according to example embodiments of the invention. The blood sample can be a whole blood sample or a plasma sample. According to the method, a coagulation factor is added to plural aliquots of the blood sample. Each aliquot can receive the coagulation factor at a different concentration. The plural aliquots can be applied to plural channels of a microfluidic device. Alternatively, or in addition, the coagulation factor(s) can be pre-coated on or into the device to which the blood sample is applied. Clot formation times are measured in each of the channels and coagulation is assessed based on the clot formation times measured. Alternatively, or in addition, degree of clot formation (optionally, degree of clot dissolution) in each of the channels is measured at a fixed time or times, and coagulation is assessed based on the degree of clot formation (optionally, degree of clot dissolution) measured.

Optionally, as illustrated in FIG. 6, the clot formation times can be compared to a reference value or reference ranges. In one example, the clot formation times are compared to coagulation factor specific clot formation reference ranges from individuals who do not suffer from a coagulation cascade abnormality. This is useful, e.g., to detect a coagulation cascade abnormality in the blood sample. In another example, the clot formation times are compared to clot formation times measured for a sample from an individual who does not suffer from a coagulation cascade abnormality. This is also useful, e.g., to detect a coagulation cascade abnormality in the blood sample. In yet another example, the clot formation times are compared to clot formation times measured for a sample containing a known amount of an anticoagulation agent. This is useful, e.g., to detect the anticoagulation agent in the blood sample.

The microfluidic device for use in the method of FIG. 6 can be any microfluidic device described herein having plural channels, such as the devices illustrated in FIGS. 1A-1D, 2A-2B, 3A-3C, 4A-4B and 5A-5B. In an embodiment, the device includes plural channels formed in a substrate, each channel including a clot forming area having a geometry configured to trigger and/or localize formation of a clot, the clot forming areas of the plural channels being arranged in a central region of the substrate; plural input ports, each input port connected to a first end of one of the plural channels; and plural output ports, each output port connected to a second end of one of the plural channels, the input and output ports being arranged in an alternating pattern at a periphery of the substrate.

### Example 7

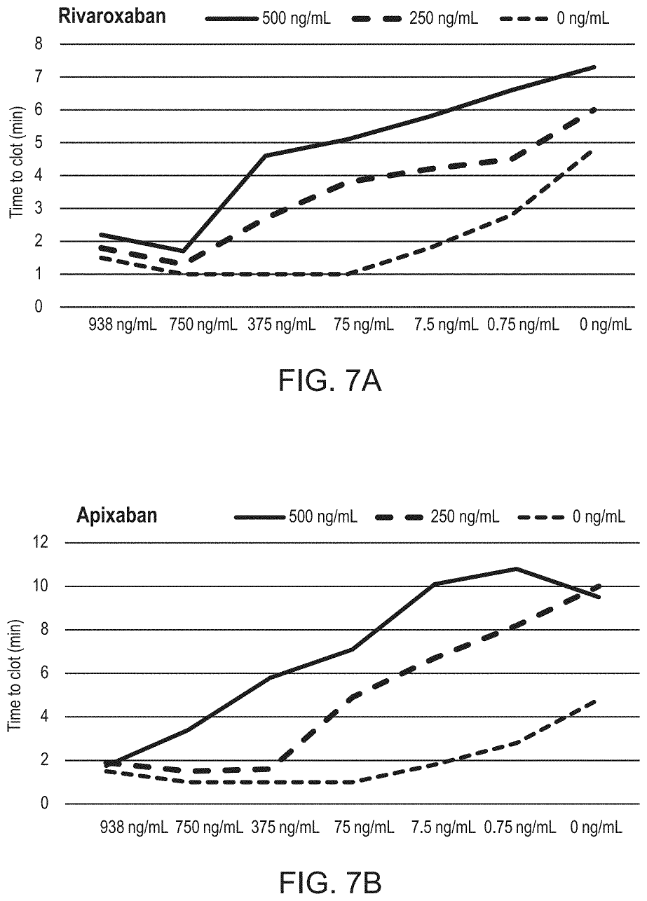

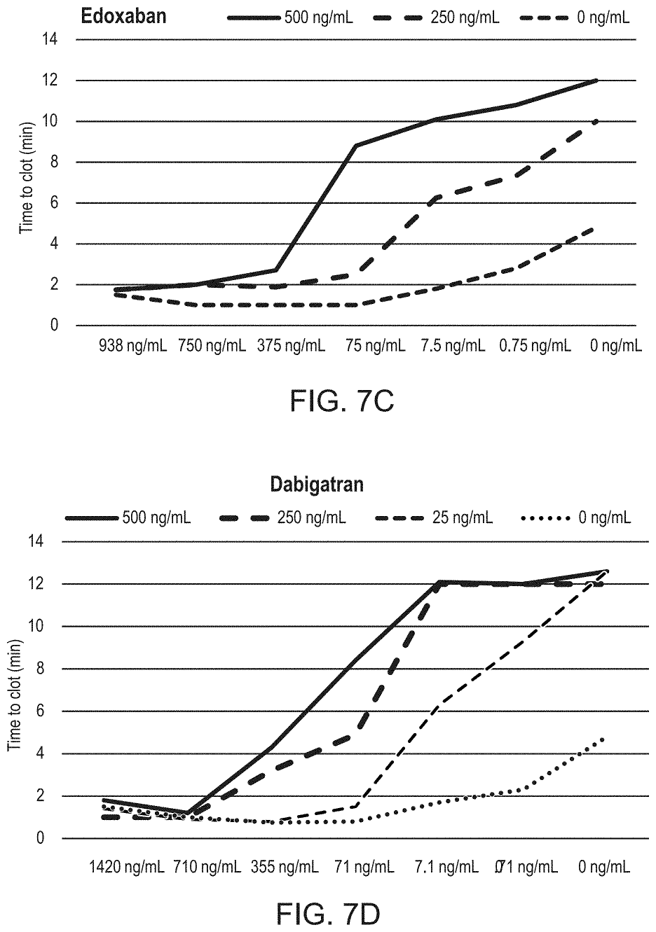

FIGS. 7A-7D illustrate example clotting curves for various FXa and FIIa inhibitors at various concentrations. The time it takes for each of the combinations to form a clot is then plotted. The clotting curve for each concentration of inhibitor is dependent on the presence and concentration of the anticoagulant in the sample. The figures illustrate the time-to-clot for four (4) different DOACs when exposed to agonists at various concentrations. The time-to-clot increases as the concentration of the inhibitor increases, demonstrating an increase in functional anticoagulation. Concentration of the agonist (FXa for FIGS. 7A-7C, and FIIa for FIG. 7D) is plotted on the X-axis for each of the figures. The units on the X-axis “ng/mL” refer to ng×10³/mL.

FIG. 7A is a graph of example data illustrating detection of Rivaroxaban. The graph shows clotting curves for different concentrations of the inhibitor Rivaroxaban (0 ng/mL, 250 ng/mL, and 500 ng/mL). Each curve shows average clot detection time (minutes; y-axis) as a function of agonist (FXa) concentration (ng×10³/mL; x-axis). The data shown in the graph can be summarized as follows:

At a concentration of 0 ng/mL Rivaroxaban, clot formation detected in <2.5 minutes with agonist concentration down to 7.5 ng×10³/mL.

At a concentration of 250 ng/mL Rivaroxaban, clot formation time is significantly longer than the negative control but lower than 500 ng/mL with agonist concentration down to 375 ng×10³/mL.

At a concentration of 500 ng/mL Rivaroxaban, clot formation detected<2.5 minutes down to 750 ng×10³/mL.

FIG. 7B is a graph of example data illustrating detection of Apixaban. The graph shows clotting curves for different concentrations of Apixaban (0 ng/mL, 250 ng/mL, and 500 ng/mL). As in FIG. 7A, each curve shows average clot detection time (minutes; y-axis) as a function of agonist (FXa) concentration (ng×10³/mL; x-axis). The data shown in the graph can be summarized as follows:

At a concentration of 0 ng/mL Apixaban, clot formation detected in <2.5 minutes with agonist concentration down to 7.5 ng×10³/mL.

At a concentration of 250 ng/mL Apixaban, clot formation detected in <2.5 minutes with agonist concentration down to 75 ng×10³/mL.

At a concentration of 500 ng/mL Apixaban, clot formation detected in <2.5 minutes with agonist concentration down to 938 ng×10³/mL.

FIG. 7C is a graph of example data illustrating detection of Edoxaban. The graph shows clotting curves for different concentrations of Edoxaban (0 ng/mL, 250 ng/mL, and 500 ng/mL). As in FIG. 7A, each curve shows average clot detection time (minutes; y-axis) as a function of agonist (FXa) concentration (ng×10³/mL; x-axis).

FIG. 7D is a graph of example data illustrating detection of Dabigatran. As in FIGS. 7A and 7B, the graph of FIG. 7D shows clotting curves for different concentrations of the inhibitor, here Dabigatran (0 ng/mL, 25 ng/mL, 250 ng/mL, and 500 ng/mL). Each curve shows average clot detection time (minutes; y-axis) as a function of agonist (FIIa) concentration (ng×10³/mL; x-axis). The data shown in the graph of FIG. 7D can be summarized as follows:

At a concentration of <25 ng/mL Dabigatran, clot formation detected in <2.5 minutes with agonist concentration down to 71 ng×10³/mL.

At a concentration of 250 ng/mL Dabigatran, get clot formation detected in <2.5 minutes with agonist concentration down to 710 ng×10³/mL.

At a concentration of 500 ng/mL Dabigatran, clot formation detected in <2.5 minutes down to 710 ng×10³/mL.

Automation can be employed to reduce variation between samples and assays.

### Example 8

In addition to the detection of the presence of FXa inhibitors and estimation of their relative concentrations, the assay described here can differentiate FXa inhibitors from FIIa inhibitors by selecting appropriate upstream and downstream clotting factors to add to the samples.

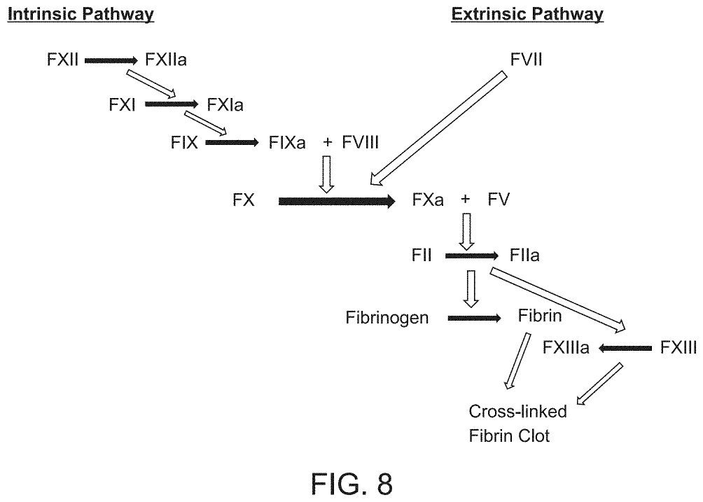

FIG. 8 illustrates a basic clotting cascade that can guide the selection of appropriate clotting factors, as further described in the following examples. As shown in FIG. 8, the cascade includes an intrinsic pathway and an extrinsic pathway, both of which can lead, via a common pathway of the cascade, to a cross-linked Fibrin clot. The intrinsic pathway can, for example, be activated by surface contact. The extrinsic pathway can be activated, for example, by tissue trauma.

### Example 9

FIG. 9 is a schematic diagram providing a demonstration of how to detect Factor Xa (FXa) inhibition/deficiency/abnormality of function. The addition of upstream (not active or activated) coagulation factors, including but not limited to FXII, FXI, FIX, FVIII, will demonstrate prolongation of clotting time, e.g., as compared to addition of a downstream factor. Alternatively, prolongation of clotting time can be determined with reference to a control clotting time. However, the addition of downstream (activated) coagulation factors, including FIIa will demonstrate unaffected (e.g., normal) clotting time, which can serve as a control. The addition of FXa will demonstrate prolongation of clotting time in a concentration-dependent manner, and even at high concentration of the upstream factor, the clotting team will likely not reach the control. As illustrated, example direct FXa inhibitors include Rivaroxaban, Apixaban, Edoxaban, and Betrixaban.

### Example 10

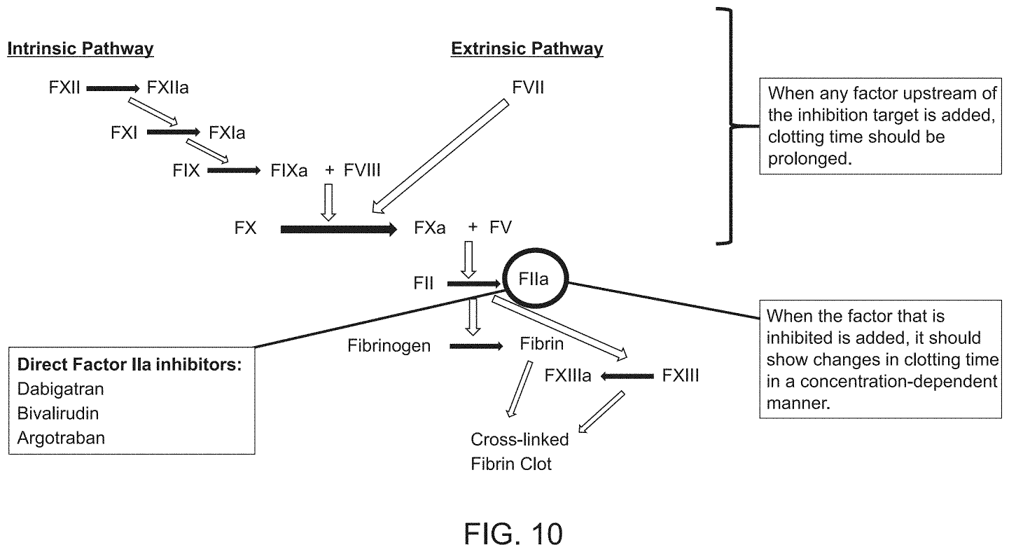

FIG. 10 is a schematic diagram providing a demonstration of how to detect Factor IIa (FIIa) inhibition/deficiency/abnormality of function. The addition of upstream (not active or activated) coagulation factors, including but not limited to FXII, FXI, FIX, FX, FV, FVIII, will demonstrate prolongation of clotting time. The addition of FIIa will demonstrate prolongation of clotting time in a concentration-dependent manner. As illustrated, example direct FIIa inhibitors include Dabigatran, Bivalirudin, and Argotraban.

### Example 11

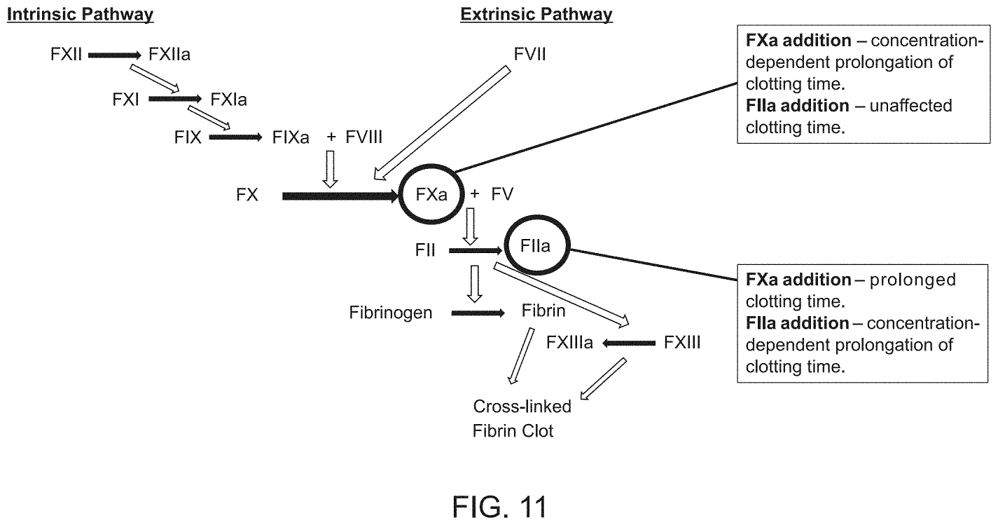

FIG. 11 is a schematic diagram providing a demonstration of how to detect and differentiate between FIIa and FXa inhibition in a sample. In the presence of a FXa and FIIa inhibitor, addition of upstream (not active or activated) coagulation factors, including but not limited to FXII, FXI, FIX, FVIII, will demonstrate prolongation of clotting time. The addition of FXa to the sample will demonstrate prolongation of clotting time, in a concentration-dependent manner for FXa and FIIa inhibition. The addition of FIIa to the sample will demonstrate prolongation of clotting time, in a concentration-dependent manner in the presence of FIIa inhibition but will demonstrate unaffected clotting time in the presence of FXa inhibition.

### Example 12

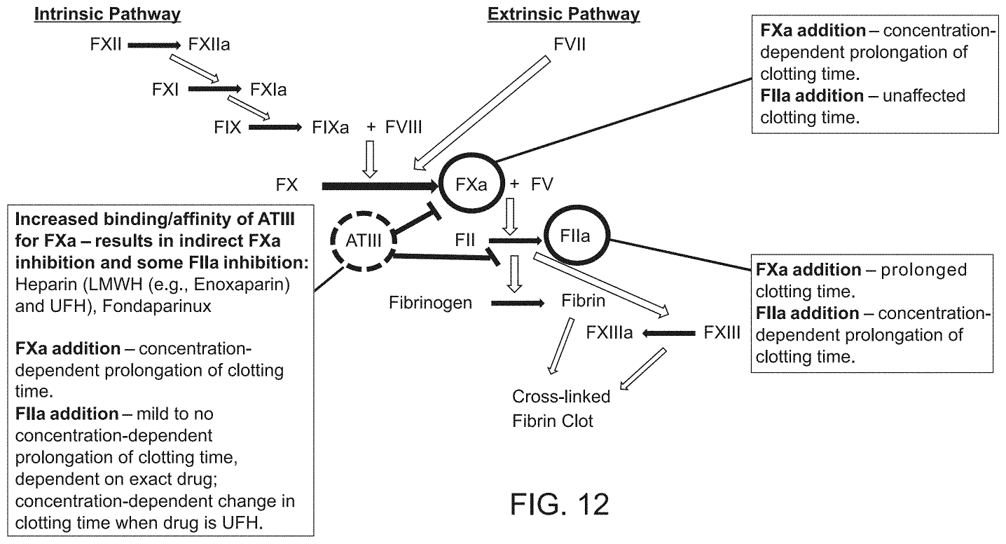

FIG. 12 is a schematic diagram providing a demonstration of how to detect indirect FXa impairment of function, such as by drugs that interact with ATIII. The addition of upstream (not active or activated) coagulation factors, including but not limited to FXII, FXI, FIX, FVIII, will demonstrate prolongation of clotting time. The addition of downstream (activated) coagulation factors, including FIIa, will demonstrate normal clotting time or prolongation of clotting time in a concentration-dependent manner dependent on the type of inhibitor present. The addition of FXa will demonstrate prolongation of clotting time in a concentration-dependent manner. The inhibition of FXa and/or FIIa is due to secondary FXa and/or FIIa inhibition via the presence of a drug that increases the affinity/binding of Antithrombin III (ATIII) to FXa and/or FIIa, thereby inhibiting it. Embodiments can include detection of ATIII, thereby detecting indirect inhibition of FXa, FIIa, or both. Drugs that increase binding/affinity of ATIII for FXa and/or FIIa include Heparin, e.g., Low Molecular Weight Heparin (LMWH) (e.g., Enoxaparin) and Unfractionated Heparin (UFH), and Fondaparinux.

### Example 13

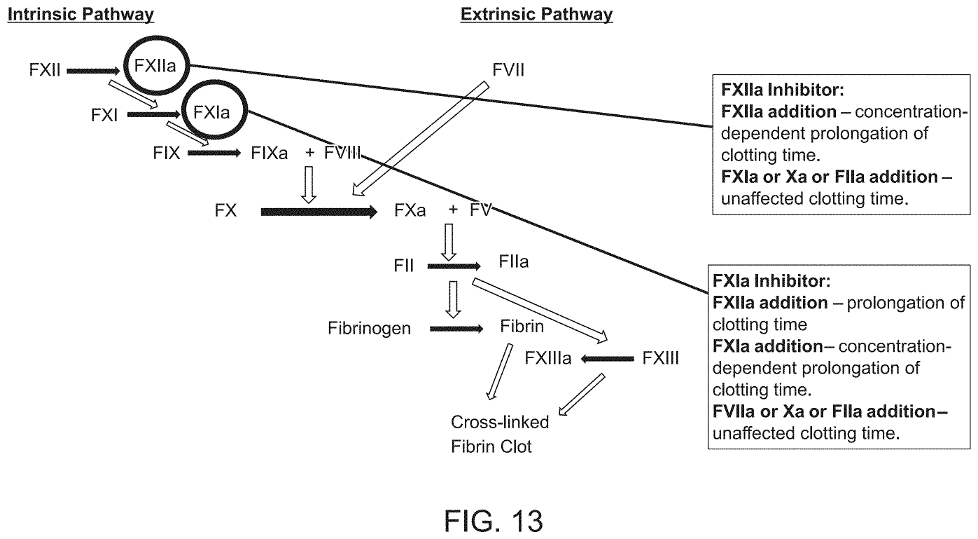

FIG. 13 is a schematic diagram providing a demonstration of how to detect and differentiate between FXIIa and FXIa inhibition in a sample. In the presence of a FXIIa inhibitor, addition of FXIIa will result in concentration-dependent prolongation of clotting time. The addition of a downstream factor in its activated form, including but not limited to, FXIa, FIXa, FVIIIa, FXa, FIIa, FVa, would result unaffected clotting times. In the presence of a FXIa inhibitor, addition of FXIIa will result in prolongation of the clotting time. Addition of FXIa would result in a concentration-dependent prolongation of clotting time. The addition of an activated downstream factor, including but not limited to, FIXa, FVIIIa, FXa, FIIa, FVa, would result unaffected clotting times. This approach can also be used in various combinations to perform a comprehensive panel for the detection and differentiation of FXIIa inhibitors, FXIa inhibitors, FXa inhibitor, and FIIa inhibitors.

### Example 14

FIG. 14 is a schematic diagram illustrating how to detect and differentiate between various types of hemophilia. Note that FXIa, although appearing to be upstream of FVIII, may, at high enough concentrations, bypass FVIII in the coagulation cascade. As shown in the diagram, a sample from a patient with Hemophilia C will have an impairment in Factor XI and will thereby exhibit a clotting time that is prolonged relative to the clotting time of a normal sample or reference sample representing normal clotting times. The clotting time of such a sample would remain prolonged with the addition of an upstream factor such as FXIIa (and would remain unchanged relative to a negative control patient sample with no exogenous factor added), would exhibit a concentration-dependent decrease in clotting time with the addition of FXI (decrease in clotting time dependent on the concentration of FXI added), and would exhibit unaffected or normal clotting times with the addition of FXIa or any other downstream activated factor. A sample from a patient with Hemophilia B will have an impairment in Factor IX and would exhibit a clotting time that is prolonged compared to the clotting time of a normal sample. For such a sample with Hemophilia B, the clotting time would remain prolonged with the addition of FXIIa or FXIa (and would remain unchanged relative to a negative control patient sample with no exogenous factor added), would exhibit a concentration-dependent decrease in clotting time with the addition of FIX, and would exhibit a normal or unaffected clotting time (relative to a normal or reference clotting time) with the addition of FIXa or any other downstream activated factor. A sample from a patient with Hemophilia A will have an impairment in Factor VIII and will exhibit a clotting time that is prolonged compared to the clotting time of a normal or reference sample. For this sample from a patient with Hemophilia A, the clotting time would remain prolonged (relative to a normal sample, and would remain unchanged relative to a negative control sample with no exogenous factor added) with the addition of FXIIa, would exhibit a concentration-dependent decrease in clotting time with the addition of FVIII (decrease in clotting time dependent on the concentration of FVIII added), and would exhibit a normal or unaffected clotting time with the addition of FXa or any other downstream activated factor. In addition, because FXIa has been shown to bypass FVIII at increased concentrations, the addition of FXIa to such a sample may result in a concentration-dependent decrease or normalization of clotting time.

For congenital disorders where a subject has a factor deficiency, the activated form of the factor that is deficient (or, alternatively, the activated form of another factor that is downstream of the deficient factor) can serve as the downstream factor in the assay; addition of the activated form of the deficient factor (or the addition of the activated form of another factor that is downstream of the deficient factor) would result in unaffected clotting time (clotting time that is not prolonged, such as clotting time that is or approaches the clotting time of normal plasma or the clotting time of a reference range). In such cases, addition of the factor that is deficient can be used to detect the deficiency, while the activated form of the deficient factor (or the activated form of a different downstream factor) can serve as the positive control to confirm the ability of the sample to form a clot.

### Example 15

FIG. 15 is a schematic diagram illustrating how to detect a fibrinogen (Factor I) impairment (such as a fibrinogen deficiency, including deficiencies caused by an abnormality) or FXIII impairment. A sample with a fibrinogen deficiency (e.g., afibrinogenemia or dysfibrinogenemia) would exhibit prolonged clotting time with the addition of any factor upstream of FI (including FIIa) (depending on the sensitivity of the clot detection methodology, no clot may be detected during the testing time), and would exhibit concentration-dependent prolongation with the addition of FI.

There is a critical concentration of functional fibrinogen necessary for the formation of a cross-linked fibrin clot that can be detected. The absolute concentration of functional fibrinogen required for clot formation detection may depend on the methodology used for clot detection (e.g., a sensitive clot detection method may detect a weak, low-fibrinogen clot, whereas a less sensitive methodology may require a higher amount of functional fibrinogen to produce a larger and stronger clot that can be detected). If there is a low amount of functional fibrinogen in the test sample, adding exogenous fibrinogen (at an amount reaching the minimum concentration required by the detection methodology to detect a clot), should result in clot formation in the sample, and depending on clot detection sensitivity, the addition of fibrinogen may result in a concentration-dependent change in clotting time. Adding FIIa in conjunction with fibrinogen (FI) to a single test sample portion serves as the positive control for this test and controls for any other impairment that may be present upstream of fibrinogen, including the presence of a thrombin inhibitor or heparin. In a sample that has low functional fibrinogen or dysfunctional fibrinogen, testing for clot formation upon the addition of exogenous FI and FIIa to the sample will reveal whether there is a fibrinogen impairment. Unlike the dilute thrombin time test (dTT), which is used to detect thrombin inhibition and fibrinogen deficiency or abnormality but is unable to differentiate between thrombin and fibrinogen impairment, this testing approach provided herein allows for the detection of fibrinogen deficiency even in the presence of other inhibitors (such as thrombin inhibitors) that may prolong or prevent coagulation. In order to account for the inhibition due to thrombin inhibitors, a concentration of FIIa sufficient to overcome all FIIa inhibition is required.

FXIII deficiency/abnormality would result in changes in clot strength and clot stability and retraction over time with the addition of a factor upstream of FXIII, concentration-dependent changes in clot strength and stability over time with the addition of FXIII

### Example 16

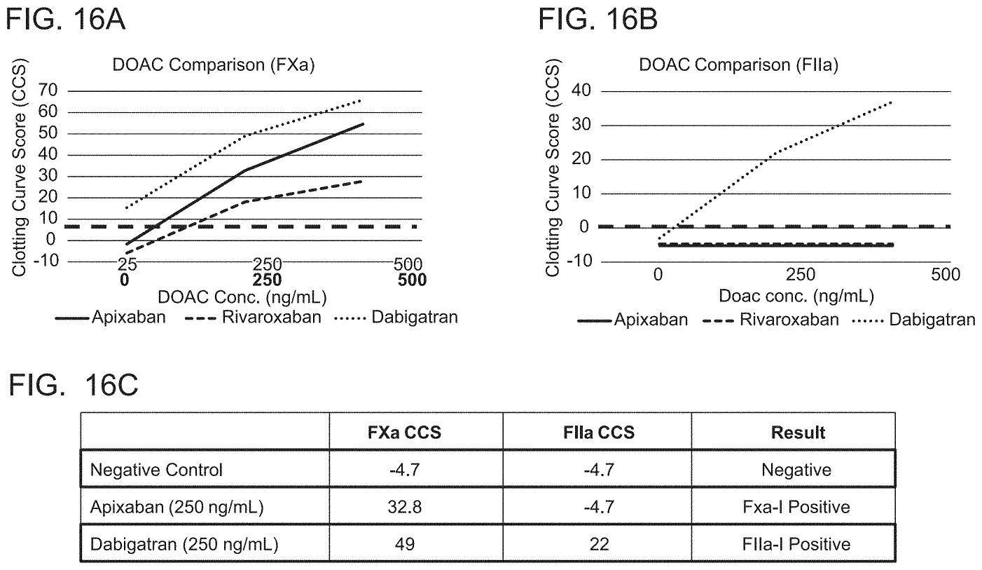

FIGS. 16A-16C illustrate Clotting Curve Scores (CCS) for FXa and FIIa inhibitors at various concentrations. Raw data of the clotting times of each of the agonists at various concentrations are used to calculate a single Clotting Curve Score (CCS) based on multivariate statistical modeling. This CCS can then be used as a single whole number to bin patients into positive or negative for specific inhibitors. This CCS can also be used to extrapolate the functional concentration of the drug in the patient sample. Functional concentration represents the amount of anticoagulation secondary to the drug in the blood sample. FIG. 16A shows how the CCS of two FXa inhibitors (Apixaban, Rivaroxaban) and one FIIa inhibitor (Dabigatran) vary dependent on concentration using FXa as the agonist. FIG. 16B shows how the CCS of the two FXa inhibitors and the one FIIa inhibitor vary dependent on concentration using FIIa as the agonist. FIG. 16C demonstrates how the CCS for each agonist can be used to identify the type of inhibitor in the sample.

### Example 17

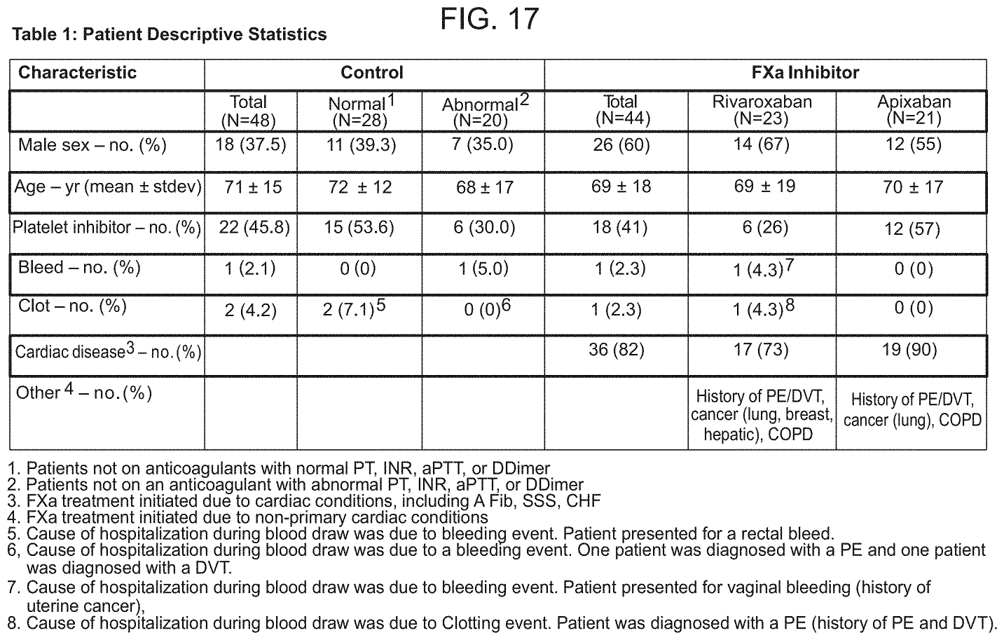

FIG. 17 shows Table 1 that provides patient descriptive statistics. Citrated plasma samples were collected from patients admitted into the Massachusetts General Hospital Emergency Department. All plasma samples had clinician-ordered coagulation tests (PT/INR, aPTT, DTT, or other). Patient samples were evaluated using an embodiment of the assay described herein. Patient medical records were reviewed for the administration of history of anticoagulants. All patient samples were collected following Institutional Review Board (IRB) approval and regulations at both the Massachusetts General Hospital and the Massachusetts Institute of Technology.

### Example 18

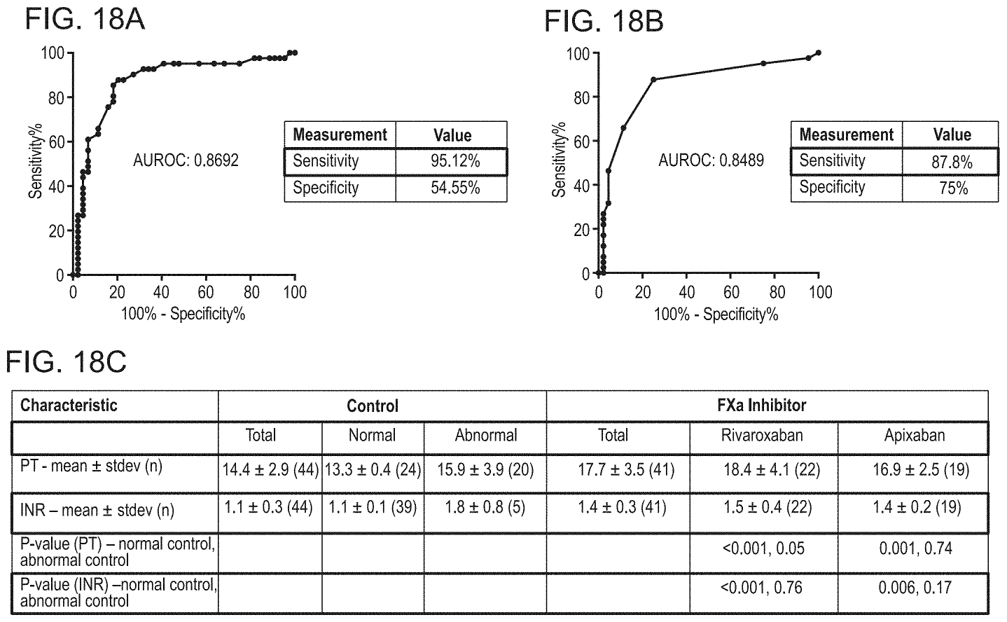

FIGS. 18A-18C illustrate prothrombin time (PT) and international normalized ratio (INR) is sensitive but not specific for FXa-I anticoagulation. Both PT and INR were compared between control patients and patients documented to be on FXa-I. Abnormal PT was defined as >14 seconds and abnormal INR was defined as >1.2. FIGS. 18A and 18B show ROC curves comparing PT and INR of total controls to patients on FXa-I. FIG. 18C shows a table of descriptive statistics of patients with PT and INR results evaluated. One-way ANOVA was used to compare normal and abnormal controls with both rivaroxaban and apixaban. Significance was defined as p<0.05. Results show that, when compared to abnormal controls, there is no significance compared to the FXa-I patients.

### Example 19

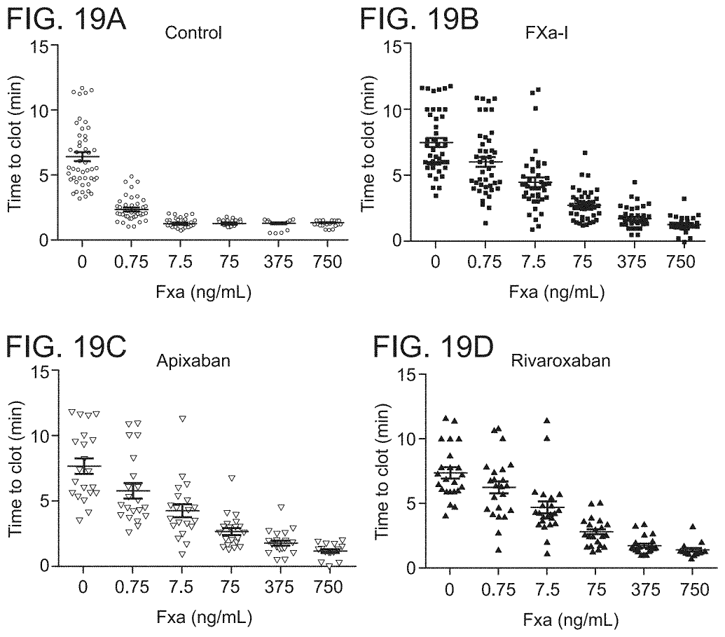

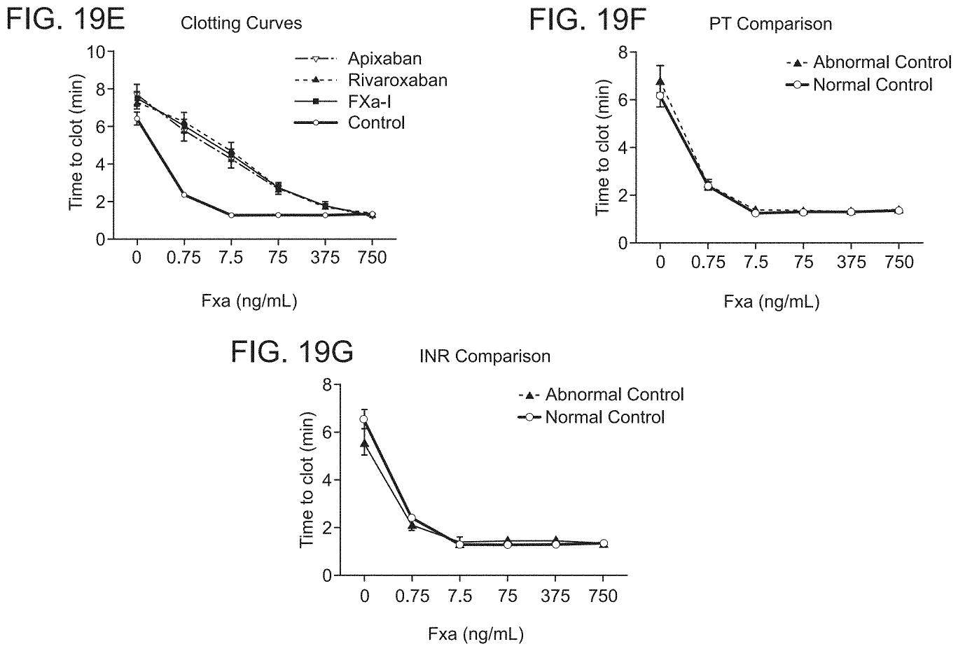

FIGS. 19A-19G illustrate example clotting time data and comparative clotting curves. Clotting times were compared at various agonist concentrations for all the patient groups to construct clotting curves. FIGS. 19A-19D show scatter plots demonstrating the mean and standard error bars of the clotting times at various agonist concentrations with respect to patients in different groups. FIG. 19E shows the mean clotting time with standard error bars of all patient groups, which are demonstrated on a single graph for comparison. All three FXa-I groups (Apixaban, Rivaroxaban, FXa-I) appear subjectively very different from the control group, with there being multiple concentrations where there are significant statistical differences between the controls and the total FXa-I, Rivaroxaban, and Apixaban groups. FIGS. 19F and 19G shows mean time to clot with standard error bars of the control group divided into patients with normal versus abnormal PT or INR, demonstrating no large difference in these tests between the different control groups.

### Example 20

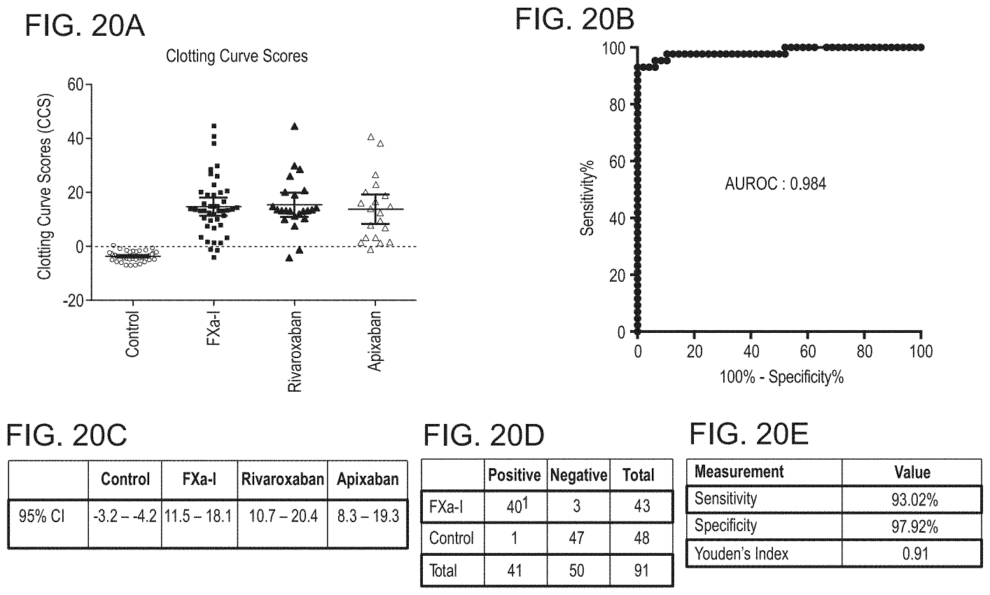

FIGS. 20A-20E illustrate Clotting Curve Score (CCS) analysis and evaluation of CCS utilization for the detection of FXa-I in patient samples. FIG. 20A shows a scatter plot with mean and standard error bars for CCS comparison between patient groups. Dotted line at CCS of 0 represents the chosen cut-off for the determination of whether there is FXa inhibition in the patient sample. FIG. 20B shows an ROC curve of utilizing the CCS scores to determine whether a patient has an FXa-I in their system. FIG. 20C provides descriptive statistics of the CCS for the different patient groups. FIGS. 20D and 20E illustrate evaluation of using the CCS for the determination of the accuracy of FXa-I detection.

### Example 21

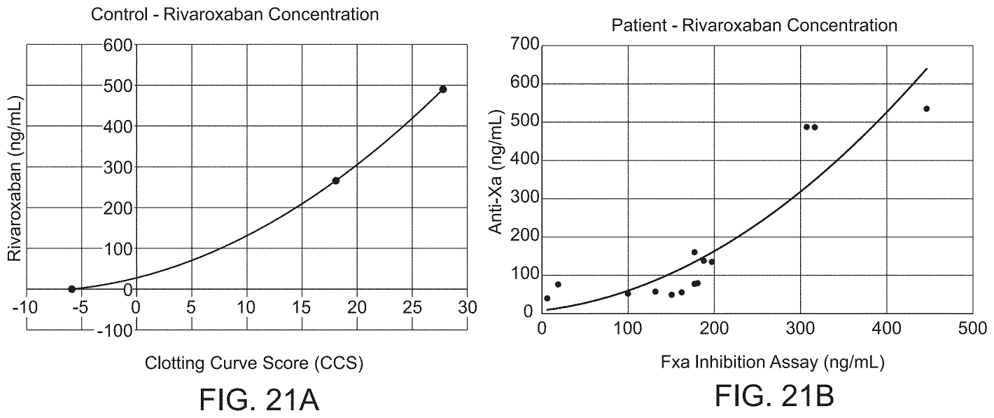

FIGS. 21A and 21B illustrate functional drug concentration calculation. Utilizing the CCS score calculated for each of the controlled spiked Rivaroxaban samples, a best-fit line was plotted for an equation that converted CCS into drug concentration, as shown in FIG. 21A. This equation was then applied to each of the CCS values for the patient samples evaluated in order to derive a functional concentration for each patient sample. These concentrations were directly compared to anti-Xa chromogenic assay-derived Rivaroxaban concentrations in each sample. Plotting these two values against each other demonstrated a good correlation between the anti-Xa concentration and the DOAC test concentration (R\hat{ }2=0.827), as shown in FIG. 21B. Note that hemolyzed samples were not included in this direct comparison because it is known that hemolyzed, icteric, and lipemic plasma samples negatively affect the anti-Xa chromogenic assay concentrations.

In addition to identifying inhibition, as illustrated in the examples of FIGS. 21A and 21B, embodiments can be used to quantify the amount of inhibition.

### Example 22

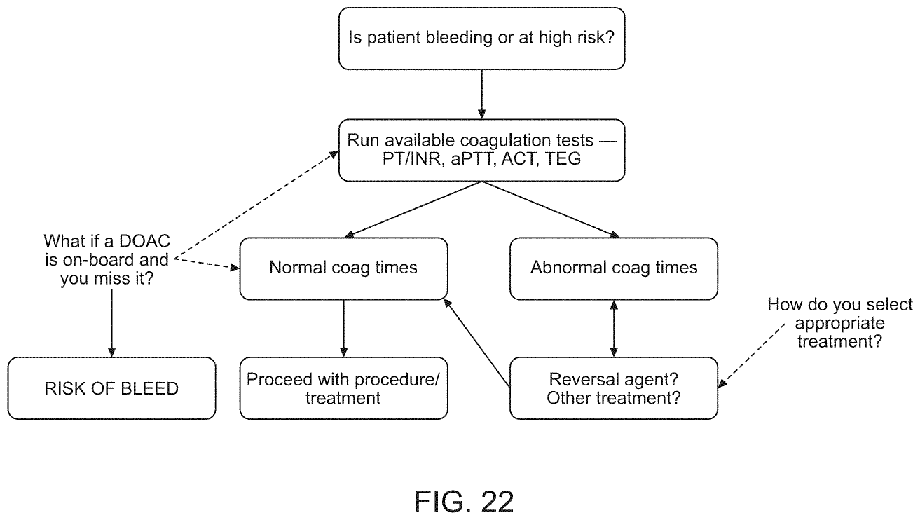

FIG. 22 illustrates a current decision-making paradigm if a patient is on a Direct Oral Anticoagulant (DOAC).

When a patient is at high-risk for a bleeding event or has an active bleed coagulation tests are ordered. These tests can include PT, INR, aPTT, ACT, TEG, or other currently available point-of-care tests. Abnormal clotting results on currently-available tests are non-specific for the presence of DOACs and leaves the healthcare worker guessing as to which treatment is the most appropriate for the patient. If the coagulation times are normal, due to the lack of sensitivity of these tests, the healthcare worker may miss the presence of a DOAC in the patient sample and proceed with treatment, putting the patient at an increased risk of bleeding.

### Example 23

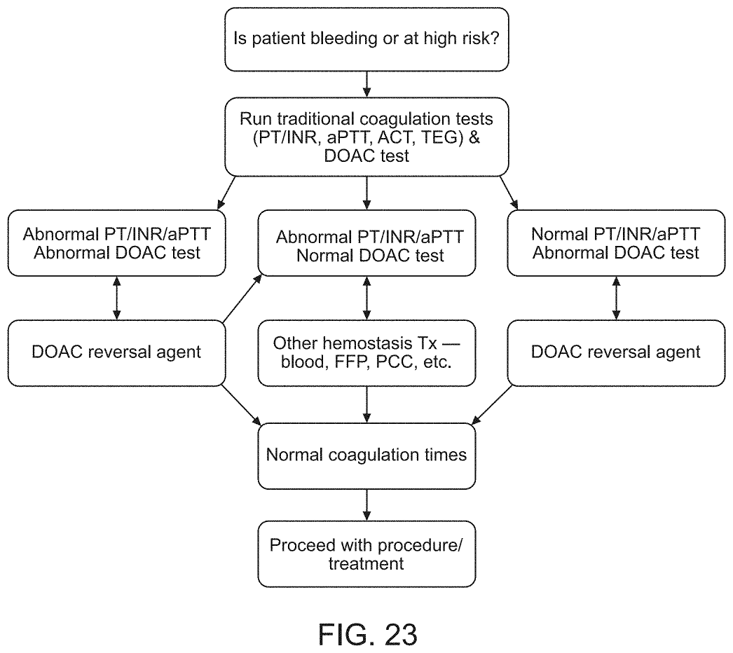

FIG. 23 illustrates a proposed decision-making paradigm using embodiment(s) of the present invention if a patient is on a DOAC. Double arrows indicate possible iterative procedures. For example, if traditional coagulation tests show a patient has normal clotting times and the DOAC test according to an embodiment of the invention shows abnormal results, a DOAC reversal agent can be selected, based on test results, and administered to the patient. The patient can then re-tested, and, if still abnormal according to the DOAC, re-tested again, optionally after administrating a modified or different DOAC reversal agent. If the traditional coagulation tests are abnormal and the DOAC test is also abnormal, then the healthcare worker may choose the DOAC reversal agent or another treatment and re-test after administration of the reagent. If the traditional coagulation tests are abnormal and the DOAC test is negative for the presence of a DOAC then the healthcare worker has the information necessary to determine that another hemostatic treatment may be necessary.

### Example 24

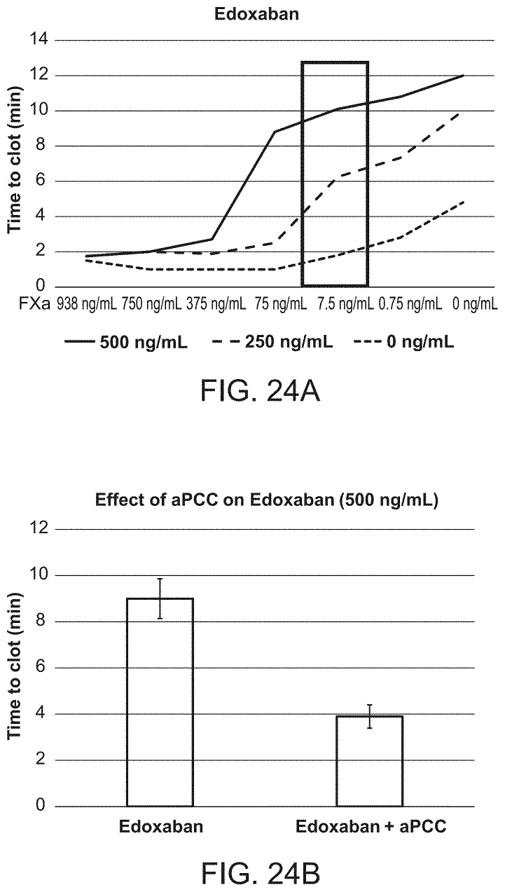

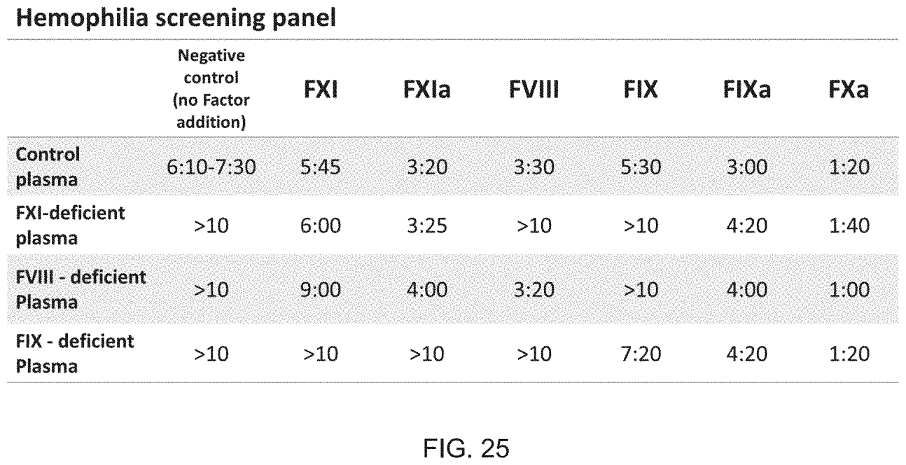

FIGS. 24A and 24B illustrate detection of the reversal of FXa inhibition following the addition of activated prothrombin complex concentrate (aPCC; FEIBA®). FEIBA® is a combination of activated factors administered to overcome FXa inhibitors in patients. Another example is Kcentra, which is inactive prothrombin complex concentrate. There are also specific FXa inhibitor reversal agents, such as coagulation factor Xa (recombinant), inactivated-zhzo. FIG. 24A demonstrates the expected clotting times for Edoxaban upon the addition of 7.5 ng×10³/mL of FXa. FIG. 24B shows the change in clotting time with a plasma sample with 500 ng/mL of Edoxaban is treated with aPCC. The data demonstrate that the test according to an embodiment of the invention has utility to monitor the reversal or overcoming of the anticoagulant effect of these DOACs.

### Example 25

FIG. 25 is a table providing representative clotting times (minutes:seconds) for various plasma samples in the presence of exogenous coagulation factors. The changes in clotting time when different coagulation factors, in active or inactive form, are added at specific concentrations to factor-deficient plasmas illustrate the upstream-downstream logic schema for using exogenous coagulation factors to identify which coagulation factor is impaired (e.g., is malfunctioning or otherwise deficient) in a patient sample. The clotting time for the control plasma, with no exogenous factor added (negative control), represents the clotting time based on activation of the contact activation pathway (intrinsic pathway); this clotting time can be designated as normal clotting time. Other clotting times represent clotting times of factor-deficient plasmas as depicted in FIG. 14, and illustrate how these clotting times change upon the addition of the inactive or active forms of specific exogenous coagulation factors. For the control (normal) plasma sample, there is a slight decrease in clotting time with the addition of exogenous FXI or FIX and a more pronounced shortening of clotting time upon the addition of FVIII, FXIa, or FIXa. The FXI-deficient plasma exhibits a prolonged clotting time (relative to the control clotting time) in the negative control condition, and with the addition of exogenous FVIII or FIX. The clotting time decreases with the addition of FXI, and decreases to a greater extent with the addition of FXIa or FIXa. The FVIII-deficient plasma exhibits a prolonged clotting time (relative to the control clotting time) in the negative control condition, and with the addition of exogenous FXI or FIX. The clotting time of the FVIII-deficient plasma decreases with the addition of FVIII or FIXa. The addition of FXIa, even though FXIa is upstream to FVIII, also decreases the clotting time in the FVIII-deficient plasma due to the ability of FXIa to bypass FVIII. The FIX-deficient plasma has a prolonged clotting time (relative to the control clotting time) in the negative control condition, and with the addition of exogenous FXI, FXIa, or FVIII. The clotting time of FIX-deficient plasma decreases with the addition of exogenous FIX or FIXa. In all plasma samples, exogenous FXa is used as a positive control to confirm the ability of the plasma sample to clot. FIIa can also be used for this purpose.

### Example 26

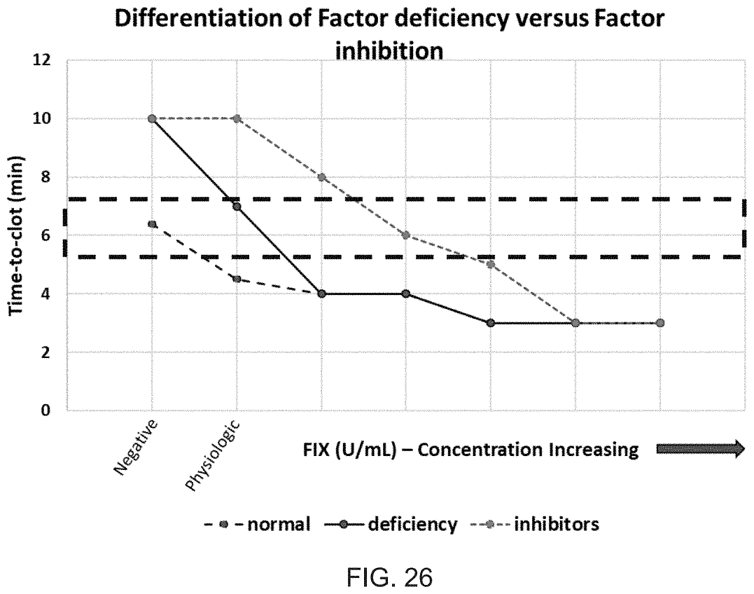

FIG. 26 is a clotting curve graph demonstrating the change in clotting time when various concentrations of Factor IX are added to Factor IX-deficient plasma (solid line, “deficiency”) or to plasma that contains anti-Factor IX antibodies resulting in Factor IX inhibition (dotted line, “inhibitors”). Both the FIX-deficient and FIX-inhibitor samples exhibit prolonged clotting times as compared to the clotting time of control plasma (slashed line, “normal”) when no Factor IX is added. In this example, the factor-deficient sample requires a physiological concentration of replacement factor in order to exhibit a normal clotting time (depicted as the range of clotting times within the dashed rectangle). The factor-inhibitor sample, on the other hand, requires supra-physiological concentrations of the factor to decrease clotting time. This difference in the concentrations of FIX required to reduce clotting time demonstrates how a difference in clotting curves can distinguish between factor-deficient and factor-inhibitor samples. It should be understood that the use of exogenous factor addition to a plasma sample may not be able to differentiate between deficiency and low quantities of inhibitors, for example, if the concentration of the inhibitor is low enough such that a physiological level of the factor is able to overcome the functional inhibition resulting in a normalized clotting time. Additionally, the inhibitor detected using the clotting time approach is a functional inhibitor such that the inhibitor decreases the factor's ability to propagate a blood clot. In some cases, patients may have non-functional coagulation factor antibodies present that do not result in changes in clotting time.

### Example 27

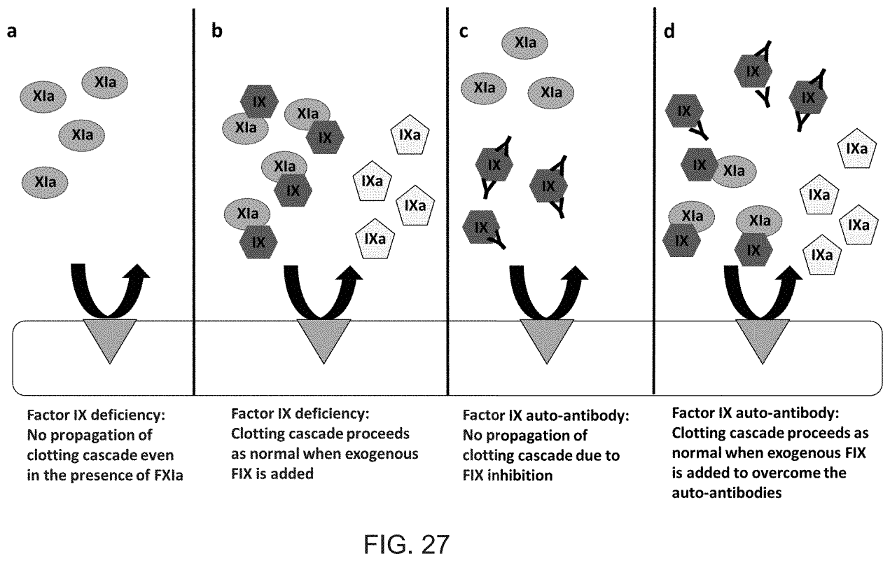

FIG. 27 is a schematic illustration of how clotting curves are used to distinguish between a factor-deficient and a factor-inhibitor sample, using Factor IX as an example. As shown in panel (a), even in the presence of FXIa, a sample deficient in FIX will not exhibit downstream coagulation. However, as illustrated in panel (b), when exogenous Factor IX is added, FXIa is able to activate FIX to form FIXa. For a FIX-deficient sample, only physiological levels of exogenous FIX are required for FIX to complex with FXIa, such that FIX will be activated to FIXa. If a sample contains FIX inhibitor that inhibits the Factor IX that is present, clotting cannot proceed, as shown in panel (c). For such a sample with FIX inhibitors (e.g., Factor IX auto-antibodies), if the concentration of exogenous FIX that is added is insufficient to overcome the level and activity of the FIX inhibitor, no FIXa will be generated. Panel (d) illustrates how the addition of a sufficiently high concentration of FIX to the FIX-inhibitor sample is required to overcome the inhibitor that is present, such that there is sufficient non-inhibited FIX that can complex with FXIa and be activated into FIXa by FXIa, leading to downstream coagulation. This schematic illustrates how, depending on the cause of Factor IX impairment (deficiency versus inhibition) and on the level and activity of any inhibitor that is present, the addition of exogenous FIX will have different effects at difference concentrations.

### Example 28

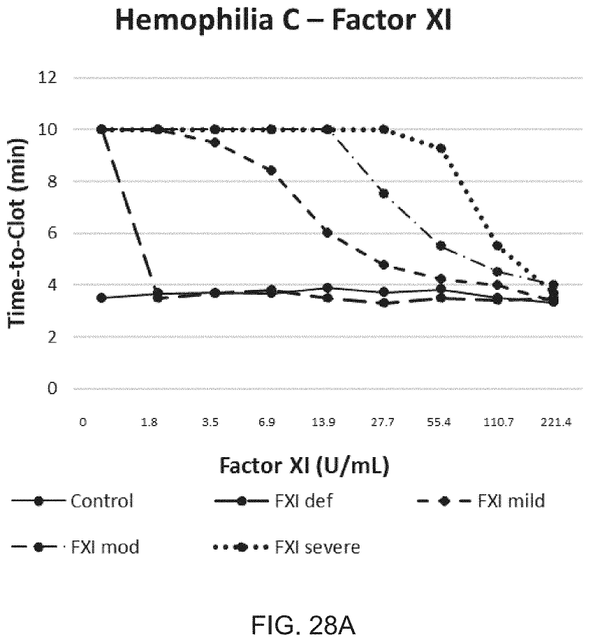

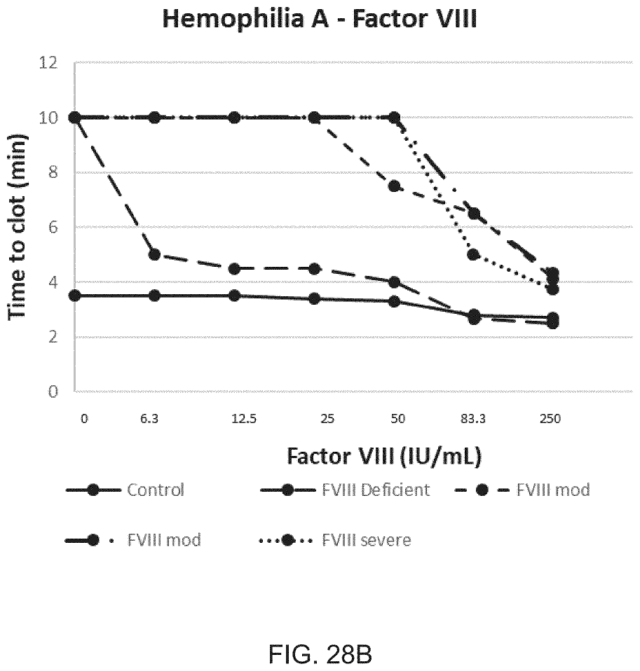

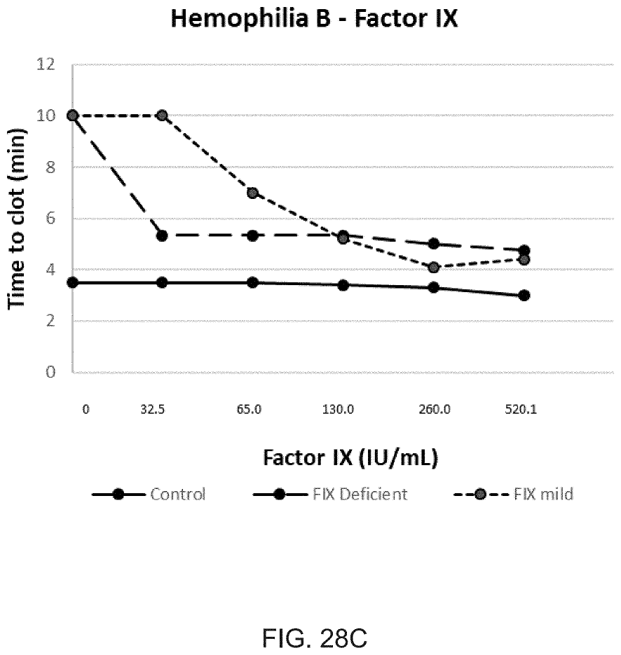

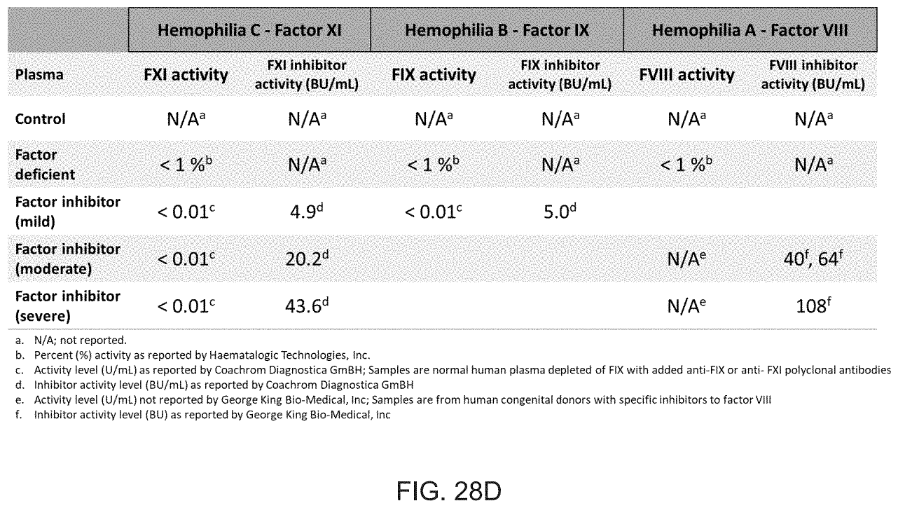

FIGS. 28A-28C are representative clotting curves demonstrating the change in clotting times as exogenous coagulation factors are added to Hemophilia C (FXI-deficient) and FXI-inhibitor samples (FIG. 28A), Hemophilia A (FVIII-deficient) and FVIII-inhibitor samples (FIG. 28B) and Hemophilia B (FIX-deficient) and FIX-inhibitor samples (FIG. 28C). The factor-deficient plasma samples exhibit shortened clotting times upon the addition of low (physiological or near-physiological) concentrations of the deficient factor, whereas the samples with factor inhibitors exhibit a concentration-dependent change in clotting time, where generally higher concentrations of factor are required to reduce clotting times for samples with “moderate” and “severe” levels of inhibition when compared to samples with a “mild” level of inhibition (see FIG. 28A and FIG. 28B). The clotting curves show the differences in clotting time when various amounts of replacement exogenous factor are added to factor-deficient and factor-inhibitor (mild, moderate, and severe) samples, demonstrating the ability to differentiate between deficiency and inhibition using the clotting curves. Additionally, the clotting curves can be used to distinguish samples having different levels of inhibition (mild, moderate, and severe levels of inhibition), as the curves shift to the right as the amount of inhibition increases, as shown in FIG. 28A and FIG. 28B. Mild, moderate, and severe inhibitor concentration was determined by Bethesda assay analysis.

In the Factor-XI inhibitor example (FIG. 28A), the FXI antibody inhibitors are man-made and were spiked into the FXI-deficiency sample at various concentrations, providing multiple plasma samples with various concentrations of the same inhibitor with the same inhibitor activity profile. This design is in slight contrast to the Hemophilia A samples (FIG. 28B), which are patient plasma samples with naturally-occurring inhibitors. As demonstrated by this assay, although there are moderate and severe concentrations of the inhibitor (as determined by the Bethesda assay), both inhibitor concentrations result in a similar shift upwards in the clotting curve. This similarity is likely due to the natural variance in antibody profiles and inhibitor activity levels. Additionally, although the clotting time significantly decreases upon the addition of a supra-physiological level of exogenous FVIII, such FVIII is not enough to completely normalize the clotting time. This observation may be due to a number of reasons, such as the potential that the antibody is also causing slight inhibition in a factor downstream of FVIII, or the possibility that the patients with inhibitors may have a different baseline clotting time compared to the baseline clotting time of the patient with the factor deficiency.

FIG. 28D is a table providing descriptive information on the plasmas of FIGS. 28A-28C, including factor activity levels and factor inhibitor levels (as determined by the Bethesda assay).

### Example 29

FIG. 29 is a diagram illustrating how to detect Factor V Leiden disease by employing coagulation factor gradients. Factor V Leiden (FVL) disease is a thrombophilia where an abnormality in FV results in a resistance to activated protein C (APC), a naturally occurring anticoagulant. As shown in the diagram, the addition of APC to a sample from a patient with FVL disease would not affect clotting time as compared to a normal sample because the exogenous APC cannot act upon FV and result in anticoagulation. Antithrombin III (ATIII) is a FXa and FIIa inhibitor and for a normal sample results in anticoagulation (like APC). The anticoagulation activity of ATIII can be increased with the addition of heparin. The addition of ATIII to a sample with FVL would result in a concentration-dependent prolongation of the clotting time, in contrast to the addition of exogenous APC (which would not affect clotting time). The clotting time observed with the addition of ATIII serves as a control for the test in the detection of anticoagulation via prolongation of the clotting time. An additional step that can be performed is the addition of exogenous normal FV, in addition to the exogenous APC, in order to demonstrate correction of the APC resistance as a confirmatory step. Another variation of this test includes the addition of exogenous Protein S, which acts as a co-factor to APC; for a normal sample, the addition of exogenous Protein S would result in a prolongation of clotting time. For a sample from a patient with FVL disease, the simultaneous addition of exogenous normal FV and Protein S should result in APC-directed anticoagulation and prolongation in clotting time, accounting for counter-effects on clotting time due to the exogenous FV.

### Example 30

The methods and devices described herein can also be used to detect hyperfibrinolysis or hypofibrinolysis. Fibrinolysis refers to the degradation of a cross-linked fibrin clot. FIG. 30 is a diagram illustrating how to detect hyperfibrinolysis by employing coagulation factor gradients. A positive control containing an exogenous activated coagulation factor, such as FIIa or FXa, will ensure the formation of a blood clot as an internal control and will also establish a baseline control for the fibrinolytic response. Measurement of fibrinolytic response will depend on the type of detection/measurement technology utilized (e.g., viscoelastic testing). Certain embodiments may also include an additional positive control containing exogenous plasmin; exogenous plasmin promotes the fibrinolytic response. For purposes of this assay (and similar to the clotting time assays described above), the positive control exogenous plasmin test would establish a normal reference range, such that the fibrinolytic response measured for the test sample when exogenous plasmin is added to the test sample can be denoted as an “unaffected” fibrinolytic response. The addition of inhibitors to uPA/tPA/sPA, such as PAI-1&2, results in an inhibition of fibrinolysis in a concentration-dependent manner; such inhibition should also correlate to the concentration of active uPA/sPA/tPA in the samples. Similarly, in order to assess the inhibitors of fibrinolysis and assess a hypofibrinolytic state, excess plasmin or excess uPA/tPA/sPA can be added to assess a concentration-dependent effect on the change in fibrinolysis activity as compared to the baseline and normal reference range.

In embodiments of this example, the fibrinolysis reaction can be performed at a temperature above room temperature, e.g., at a temperature that is within the range of about 37° C. to 45° C. Performing the assay at such temperatures can increase the fibrinolysis reaction rate and thereby reduce the time required to perform the assay (e.g., from 30-40 minutes to ≤15 minutes).

### Example 31

FIG. 31 is a table providing representative clotting times (minutes:seconds) for control plasma and fibrinogen-deficient plasma, and illustrates how such clotting times can be used to detect fibrinogen deficiency (e.g., a fibrinogen abnormality), as described herein (see, e.g., Example 15, FIG. 15). The detection of fibrinogen (Factor I) deficiency is shown here by utilizing exogenous FIIa and FI in various combinations and measuring the clotting time. The negative condition does not include any exogenous coagulation factors and clotting is initiated via the contact activation pathway (intrinsic coagulation pathway). The control plasma exhibits a clotting time within the reference range in the negative control condition, and exhibits an unchanged clotting time with the addition of FI but exhibits a shortened clotting time with the addition of FIIa and FIIa+FI. The fibrinogen-deficient plasma does not form a detectable clot during the testing time (10 minutes), both in the negative control condition as well as in the FIIa condition. When FI is added to the fibrinogen-deficient sample, the clotting time is reduced and returns to the normal range; when both FI and FIIa are added to the sample, the clotting time is further shortened. As mentioned with reference to FIG. 15, the limit of detection of clot formation may depend on the clot detection methodology. In addition, certain coagulation detection methodologies that are capable of also assessing clot phenotype (such as clot firmness or strength, and clot size) may be able to quantify or further characterize the level of functional fibrinogen.

### Example 32

FIG. 32 is a schematic diagram illustrating how to detect and differentiate between Factor IIa, Factor Xa, Factor XI, and Factor XIa inhibition. If there is inhibition of FXI and/or FXIa in a sample, the addition of FXI and/or FXIa results in a concentration-dependent prolongation of clotting time (the clotting time inversely correlates with the concentration of factor that is added, such that a higher concentration of FXI and/or FXIa results in a shorter clotting time). In the case of an inhibitor that specifically targets FXI for inhibition, the addition of exogenous FXI would result in a concentration-dependent prolongation in clotting time, while the addition of exogenous FXIa would bypass the point of inhibition and normalize clotting time (and result in an unaffected clotting time with respect to a clotting time of a normal sample or normal clotting time range). In the case of an inhibitor that targets FXIa, there would be a concentration-dependent prolongation in clotting time with the addition of exogenous FXIa (clotting time would be reduced as more exogenous FXIa is added), while the addition of exogenous FXI will either result in no change in clotting time (such that clotting time remains prolonged) or in a concentration-dependent prolongation in clotting time, depending on the concentration and activity level of the FXIa inhibitor. The addition of exogenous FXa or FIIa would result in unaffected (not prolonged) clotting times. In the case of a FXa inhibitor, the addition of FXI or FXIa would result in a prolongation of the clotting time (clotting time remains prolonged), while the addition of FXa would result in a concentration-dependent prolongation of clotting time (clotting time would be reduced further as higher concentrations of FXa are added) and the addition of FIIa would result in an unaffected (not prolonged) clotting time. In the case of a FIIa inhibitor, the addition of FXIa, FXI, or FXa would result in a prolongation of clotting time (clotting time would remain prolonged relative to the clotting time of a normal sample or a normal clotting time range), while the addition of FIIa would result in a concentration-dependent prolongation in clotting time (clotting time would be further reduced as higher concentrations of FIIa are added). In some cases of FIIa inhibition, the addition of exogenous FXa may result in a concentration-dependent prolongation in clotting time, similar to what is observed with FIIa addition. In such cases, the difference between a FXa-inhibitor sample and a FIIa-inhibitor sample is that, with the FXa-inhibitor sample, the addition of exogenous FIIa results in an unaffected (not prolonged) clotting time.

### Example 33

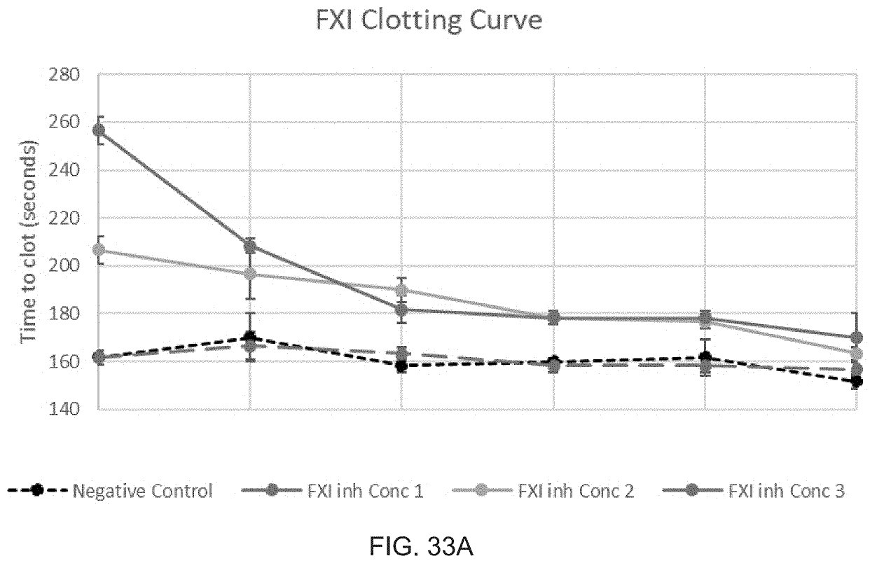

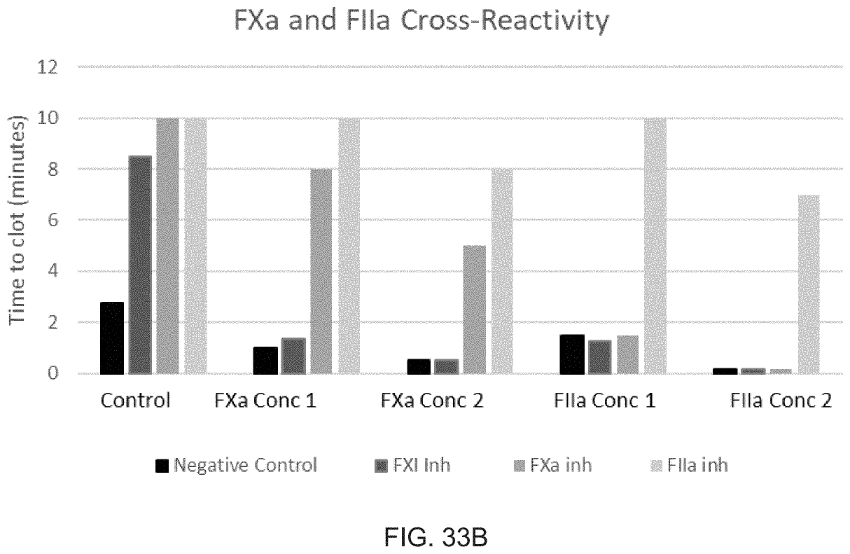

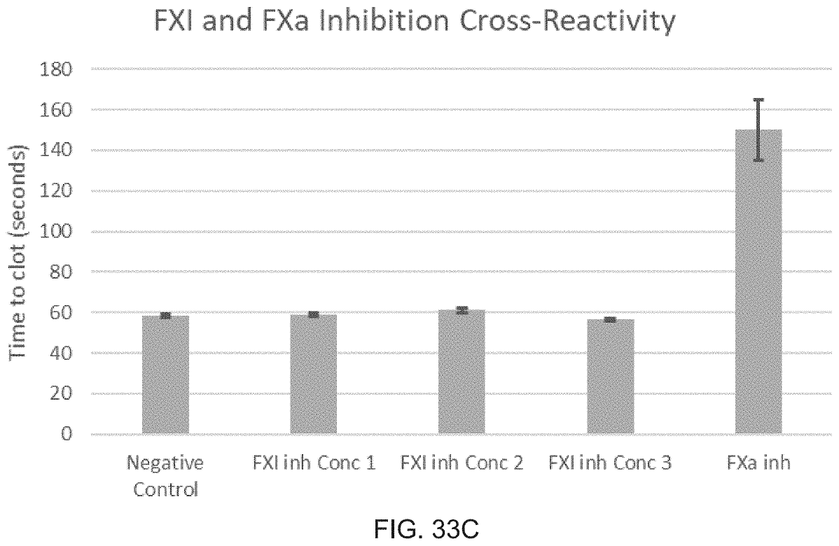

FIG. 33 illustrates embodiments for the detection of Factor XI and/or XIa inhibition and differentiation between Factor IIa, Factor Xa, Factor XI inhibition, and Factor XIa inhibition.

FIG. 33A provides representative clotting-curves of whole blood samples spiked with a concentration of FXI inhibitor (inh) (Conc 1<Conc 2<Conc 3); no inhibitor was added to the negative control. The curves show the samples' respective changes in clotting time as a response to the addition of increasing concentrations of exogenous FXI. As shown in the graph, higher concentrations of exogenous FXI are required to reduce clotting times for samples having higher concentrations of inhibitor.

FIG. 33B demonstrates the use of adding activated factors to differentiate between FXa, FIIa, and FXI inhibition in blood samples. The control condition (where no exogenous factor is added) utilizes an activator of the contact activation pathway to activate FXII to FXIIa. FIG. 33B is a bar graph showing the clotting time responses to the addition of buffer (control), two concentrations of FXa, and two concentrations of FIIa. The negative control samples, with no inhibitor, exhibited a fairly consistent and short clotting time for all conditions. The sample with the FXI inhibitor exhibited a prolongation of clotting time in the control condition (no exogenous factor added), while the clotting time decreased and normalized to reach the clotting time of the negative control sample, upon the addition of the exogenous FXa and upon the addition of exogenous FIIa. The sample with the FXa inhibitor (apixaban) exhibited a prolongation of clotting time in the control condition (no exogenous factor added), and exhibited a concentration-dependent clotting-time response to the addition of exogenous FXa and a shortened and normalized clotting time upon the addition of exogenous FIIa. The sample with the FIIa inhibitor (dabigatran) exhibited a prolongation of clotting time in the control and FXa conditions, with a relatively small concentration-dependent change in clotting time in the FXa condition, and exhibited a concentration-dependent change in clotting time with the addition of exogenous FIIa. The amount of exogenous FIIa added to the sample was inadequate to overcome all of the FIIa inhibition present in the sample. In these examples in FIG. 33B, when a sample's clotting time normalizes to the negative control clotting time, such a result is also described as an “unaffected” clotting time in the figures depicting the pathways.

FIG. 33C reports the effect of the addition of exogenous FXI on the clotting time of various samples. In this example, the negative control, containing no inhibitor, has an unaffected clotting time with the addition of FXI. The clotting time for the samples with the FXI inhibitor (Conc 1<Conc 2<Conc 3) normalize to the negative control clotting time as the concentration of FXI added fully overcomes the level of FXI inhibitor present in the sample. The sample with the FXa inhibitor (apixaban) exhibits a prolongation in clotting time, even in the presence of the concentration of exogenous FXI that was added to the sample. FIG. 33C shows that the methods of the invention can differentiate between FXI and FXa inhibition.

### Example 34

FIG. 34 is a schematic diagram illustrating how to detect and differentiate between Factor IIa, Factor Xa, Factor XI, Factor XIa, Factor XII and Factor XIIa inhibition. A sample with inhibition of FXII or FXIIa would exhibit a concentration-dependent prolongation of clotting time with the addition of exogenous Factor XIIa or FXII (clotting time would be reduced in a concentration-dependent manner, as higher concentrations of the exogenous factor are added). In the case of a sample with FXII inhibition, the sample would exhibit a prolongation of clotting time upon the stimulation of the contact activation pathway through a FXII activator, such as kaolin, celite, or glass, but there would be an unaffected clotting time (clotting time within a normal range) upon the addition of exogenous FXIIa and a concentration-dependent change in clotting time upon the addition of exogenous FXII. In the case of a sample with FXIIa inhibition, the sample would exhibit a prolongation of clotting time upon the stimulation of the contact activation pathway through a FXII activator and upon the addition of exogenous FXII; such a sample would exhibit a concentration-dependent change in clotting time upon the addition of exogenous FXIIa. For both the FXII and FXIIa inhibition samples, the addition of exogenous FXIa, FXa, or FIIa would result in an unaffected clotting time (a clotting time that is similar to normal clotting time or that falls within a normal reference range of clotting time), and the addition of exogenous FXI would result in a prolongation in clotting time (clotting time would remain prolonged). **, activation of Factor XII to FXIIa can occur via multiple activators, including specific materials, clays, charged surfaces, polyphosphates, and endotoxins as a few examples.

A sample with inhibition of FXIa or inhibition of FXI would exhibit a concentration-dependent prolongation of clotting time with the addition of FXIa or FXI (clotting time would change in a concentration-dependent manner, with higher concentrations of factor leading to further reductions in clotting time until a normal clotting time is reached). In the case of an inhibitor that specifically inhibits FXI (and does not inhibit FXIa), the addition of exogenous FXI would result in a concentration-dependent prolongation in clotting time, while the addition of exogenous FXIa would result in an unaffected clotting time (a clotting time that falls within a reference or normal clotting time range). In the case of sample containing an inhibitor that targets FXIa, the sample would exhibit a concentration-dependent prolongation or change in clotting time with the addition of exogenous FXIa at varying concentrations, while the addition of exogenous FXI would result in no response in clotting time (clotting time remains prolonged) or would result in a concentration-dependent prolongation or change in clotting time, depending on the concentration and inhibition activity of the FXIa inhibitor. The addition of exogenous FXII or FXIIa would result in no change in clotting time, such that clotting time remains prolonged, while the addition of FXa or FIIa would result in unaffected clotting times (clotting times would be unaffected relative to the clotting time of a normal sample or relative to a reference clotting time).

In the case of a sample with a FXa inhibitor, the sample would exhibit a prolongation of the clotting time upon the addition of exogenous FXII, FXIIa, FXI, or FXIa, would exhibit a concentration-dependent prolongation of clotting time with the addition of exogenous FXa (such that clotting time would decrease further upon the addition of higher concentrations of exogenous FXa), and would exhibit an unaffected clotting time (such as a normal clotting time) upon the addition of FIIa.

In the case of a sample with a FIIa inhibitor, the sample would exhibit a prolongation of clotting time upon the addition of exogenous FXIIa, FXII, FXIa, FXI, or FXa, while the addition of exogenous FIIa would result in a concentration-dependent prolongation in clotting time, as higher concentrations of exogenous FIIa would result in further reductions in clotting time. The asterisk in the figure is included to note that, in some cases of FIIa inhibition, the addition of exogenous FXa may similarly result in a concentration-dependent prolongation in clotting time. In such cases, the difference between a FXa-inhibitor sample and a FIIa-inhibitor sample is that, for the FXa-inhibitor sample, the addition of exogenous FIIa results in an unaffected clotting time (such as a normal clotting time), while for the FIIa-inhibitor sample the addition of exogenous FIIa results in a concentration-dependent change in clotting time.

While example embodiments have been particularly shown and described, it will be understood by those skilled in the art that various changes in form and details may be made therein without departing from the scope of the embodiments encompassed by the appended claims.

1-47. (canceled)

48. A method of assessing coagulation in a sample from a subject, the method comprising:
adding Factor IIa to a first portion of the sample;
adding Factor I and Factor IIa to a second portion of the sample; and
measuring clot formation for the first and second portions of the sample.

49. The method according to claim 48, further comprising:
adding Factor XI to a third portion of the sample;
adding Factor IX to a fourth portion of the sample;
adding Factor VIII to a fifth portion of the sample; and
measuring clot formation for the third, fourth, and fifth portions of the sample.

50. The method according to claim 49, further comprising:
adding Factor XII or Factor XIII to a sixth portion of the sample; and
measuring clot formation for the sixth portion of the sample.

51. The method according to claim 49, further comprising:
adding Factor IXa to a sixth portion of the sample;
adding Factor XIa to a seventh portion of the sample; and
measuring clot formation for the sixth and seventh portions of the sample.

52. The method according to claim 49, further comprising:
adding Factor XIIa to a sixth portion of the sample and measuring clot formation for the sixth portion of the sample; and
measuring clot formation for a seventh portion of the sample to which no coagulation factor has been added.

53. The method according to claim 48, further comprising:
adding Factor I to a third portion of the sample and measuring clot formation for the third portion of the sample; and
measuring clot formation for a fourth portion of the sample to which no coagulation factor has been added.

54. The method according to claim 48,
wherein the adding of Factor IIa to the first portion of the sample comprises introducing the first portion of the sample to a first channel containing Factor IIa, and
wherein the adding of Factor I and Factor IIa to the second portion of the sample comprises introducing the second portion of the sample to a second channel containing Factor I and Factor IIa, and
wherein the first channel and the second channel are channels in a microfluidics device.

55. The method according to claim 48, wherein the volume of the first portion of the sample and the volume of the second portion of the sample are each about 50 μL or less.

56. The method according to claim 48, wherein the volume of the first portion of the sample and the volume of the second portion of the sample are each about 5 μL or less.

57. The method according to any one of claims 48-53, wherein measuring clot formation comprises measuring clot formation time.

58. A method of assessing coagulation in a sample from a subject, the method comprising:
adding Factor I to two or more first portions of the sample, each first portion receiving the Factor I at a different concentration;
adding Factor IIa to two or more second portions of the sample, each second portion receiving the Factor IIa at a different concentration;
adding Factor I and Factor IIa to two or more third portions of the sample, each third portion receiving the Factor I at a different concentration and receiving the Factor IIa at the same concentration; and
measuring clot formation for each of the first, second, and third portions of the sample.

59. The method according to claim 58, further comprising measuring clot formation for one or more fourth portions of the sample to which no coagulation factor has been added.

60. The method according to claim 58, wherein the sample comprises a fluid from a subject, said fluid being whole blood.

61. The method according to claim 58, wherein the sample comprises a fluid from a subject, said fluid being plasma.

62. The method according to claim 58, wherein the volume of each of the first, second, and third portions of the sample is about 50 μL or less.

63. The method according to claim 58, wherein the volume of each of the first, second, and third portions of the sample is about 5 μL or less.

64. The method according to claim 58, wherein the Factor I is added to the two or more first portions of the sample at concentrations that differ from each other by a factor of 2 up to a factor of 20, and wherein the Factor IIa is added to the two or more second portions of the sample at concentrations that differ from each other by a factor of 5 up to a factor of 20.

65. The method according to claim 58, wherein measuring clot formation comprises measuring clot formation time.

66. A method of assessing coagulation in a blood sample, the method comprising:
adding antithrombin III and Factor Xa, or antithrombin III and Factor IIa, to a first portion of the blood sample;
adding Activated Protein C to a second portion of the blood sample; and
measuring clot formation for the first and second portions of the blood sample.

67. A method of assessing whether a patient suffers from a Factor V impairment, the method comprising:
adding antithrombin III to two or more first portions of a blood sample from the patient, each first portion receiving the antithrombin III at a different concentration;
adding Activated Protein C to two or more second portions of the blood sample, each second portion receiving the Activated Protein C at a different concentration;
adding Activated Protein C and Factor V to at least one third portion of the blood sample; and
measuring clot formation for each of the first, second, and third portions of the blood sample.

68. A method of assessing whether a patient suffers from a Factor V impairment, the method comprising:
adding antithrombin III to two or more first portions of a blood sample from the patient, each first portion receiving the antithrombin III at a different concentration;
adding Activated Protein C to two or more second portions of the blood sample, each second portion receiving the Activated Protein C at a different concentration;
adding Protein S and Factor V to at least one third portion of the blood sample; and
measuring clot formation for each of the first, second, and third portions of the blood sample.

69. The method according to any one of claims 67-68, wherein measuring clot formation comprises measuring clot formation time, and wherein the volume of each of the first, second, and third portions of the blood sample is about 50 μL or less.

70. The method according to any one of claims 67-68, further comprising adding antithrombin III and heparin to at least one fourth portion of the blood sample, and measuring clot formation for each fourth portion of the blood sample.

71. The method according to any one of claims 67-68, further comprising inputting the blood sample into a microfluidic device having separate channels for each of the first, second, and third portions of the blood sample, each separate channel comprising a location that localizes clot formation.

72. A microfluidic device for detecting coagulation in a sample from a subject, the microfluidic device comprising:
a first group of channels comprising Factor I at increasing concentrations across channels of the first group;
a second group of channels comprising Factor IIa at increasing concentrations across channels of the second group; and
a third group of channels comprising Factor I at increasing concentrations across channels, and comprising Factor IIa at the same concentration across channels of the third group,
wherein each channel in each of the first, second, and third groups of channels comprises a clot forming area that localizes clot formation, and
wherein each of the first, second, and third groups of channels comprises at least two channels.

73. The microfluidic device of claim 72, wherein the first group of channels comprising Factor I at increasing concentrations across channels comprises increasing concentrations of Factor I that differ from each other by a factor of 2 up to a factor of 20; wherein the second group of channels comprising Factor IIa at increasing concentrations across channels comprises increasing concentrations of Factor IIa that differ from each other by a factor of 5 up to a factor of 20; and wherein the third group of channels comprising Factor I at increasing concentrations and Factor IIa at the same concentration across channels comprises increasing concentrations of Factor I that differ from each other by a factor of 2 up to a factor of 20.

74. A method of evaluating coagulation factor impairment in a sample from a subject, the method comprising inputting the sample into the microfluidic device of claim 72.
